(19) United States
(12) Patent Application Publication
(10) Pub. No.: US 2025/0275953 A1
(43) Pub. Date: Sep. 4, 2025

(54) VODOBATINIB FOR REDUCING PROGRESSION OF PARKINSON'S DISEASE

(71) Applicant: Sun Pharma Advanced Research Company Ltd., MUMBAI (IN)

(72) Inventors: Siu-Long YAO, Princeton, NJ (US); Vikram Krishna RAMANATHAN, Vadodara, Gujarat (IN); Nitin Krishnaji DAMLE, Mumbai, Maharshtra (IN); Andrew GOLDFINE, Bryn Mawr, PA (US); Robert D. FAULKNER, Aledo, TX (US); Sanjay Nandlalji MANDHANE, Vadodara, Gujarat (IN); Ravi Sankar Prasad TALLURI, Navi Mumbai, Maharashtra (IN)

(21) Appl. No.: 18/858,679

(22) PCT Filed: May 2, 2023
(86) PCT No.: PCT/IB2023/054572

§ 371 Date: Oct. 21, 2024

(30) Foreign Application Priority Data
May 2, 2022 (IN) 202221025575
Nov. 1, 2022 (IN) 202221062155

(51) Int. Cl. A61K 31/47; A61K 9/00; A61K 47/02; A61K 47/20; A61K 47/26; A61K 47/32; A61K 47/34; A61P 25/16

(52) U.S. Cl. CPC A61K 31/47; A61K 9/0053; A61K 47/02; A61K 47/20; A61K 47/26; A61K 47/32; A61K 47/34; A61P 25/16

(57) ABSTRACT

The present disclosure relates to a method of treating, or delaying, inhibiting, or suppressing of the progression of, a neurodegenerative disease such as, for example, early-stage Parkinson's disease comprising administering a c-Abl inhibitor (such as vodobatinib, or a pharmaceutically acceptable salt thereof) to a subject in need thereof.

The present application is the U.S. national stage of International Patent Application No. PCT/IB2023/054572, filed May 2, 2023 which claims the benefit of Indian Provisional Application No. 202221025575, filed on May 2, 2022, and Indian Provisional Application No. 202221062155, filed on Nov. 1, 2022, the entire contents of which are hereby incorporated by reference.

### FIELD OF THE INVENTION

The present invention relates to a method of treating and/or reducing the progression of a neurodegenerative disease such as Parkinson's disease (PD), Alzheimer's disease (AD), synucleinopathies, Lewy body dementia (LBD), rapid eye movement (REM), sleep behaviour disorder, multiple system atrophy (MSA), early-stage Parkinson's disease, early stage Alzheimer's disease, early stage of synucleinopathies, early-stage lewy body dementia, early-stage rapid eye movement, early-stage sleep behaviour disorder and early-stage multiple system atrophy, in a subject (e.g., a human subject in need thereof) by administering a c-Abl inhibitor (such as vodobatinib, or a pharmaceutically acceptable salt thereof) to the subject.

### BACKGROUND OF THE INVENTION

Neurodegenerative disease (ND) occur when nerve cells in the brain or peripheral nervous system lose function over time and ultimately die. These diseases cannot be cured completely, however there are several medications used to relieve some of the symptoms associated with the diseases (Fernando et al. Pharmaceuticals. 2018, 11, 44).

For patients with advanced neurodegenerative disease, some surgical procedures are an option, however these procedures are risky for the elderly population (the populations that most often suffers from the advanced stages of neurodegenerative disease), and they do not stop the progression of the disease.

Nilotinib has been tested for treating Parkinson's disease in three random clinical trials (see, e.g., Pagan et al., JAMA Neurol., 2020; 77(3):309-317; Simuni et al., medRxiv preprint, May 12, 2020; see also Turner et al., Annals Neurology 88(1):183-194, 2020). While nilotinib showed improvement in part of cerebrospinal fluid (CSF) biomarker levels, the poor efficacy on motor outcomes indicated that nilotinib had no advantages in the clinic (Xie et al., Front Aging Neurosci. 2022, 14:996217).

Nilotinib was also shown to be unsuccessful in slowing Parkinson's disease (PD) progression (Simuni et al. JAMA Neurol., 2021; 78(3):312-320). The failure of nilotinib at its maximally permissible dose of 300 mg was attributed to insufficient brain penetration because the concentration of nilotinib in cerebrospinal fluid (CSF) was found to be 7-fold lower than its IC₅₀ for c-Abl. Simuni specifically states that the low CSF exposure, lack of biomarkers effect and efficacy data trending in the negative direction indicate that nilotinib should not be further tested in Parkinson's disease.

Vodobatinib (N′-(2-chloro-6-methylbenzoyl)-4-methyl-3-[2-(3-quinolyl) ethynyl]-benzohydrazide), a c-Abl inhibitor is represented by Formula I (referred hereinafter interchangeably as vodobatinib or compound of Formula I)

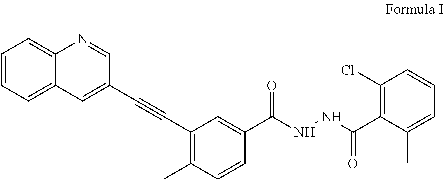 [SMILES: Cc1ccc(C(=O)NNC(=O)c2c(C)cccc2Cl)cc1C#Cc1cnc2ccccc2c1]

International Publication Nos. WO 2017/208267A1, WO 2020/250133A1 and WO 2022/024072A1, which are hereby incorporated by reference, disclose methods of use of the compound of Formula I for the treatment of Parkinson's disease, synucleinopathies and Alzheimer's disease (AD) respectively.

There is a continuing need for effective and safe methods for the treatment of, and delaying the progression of, neurodegenerative diseases, including in the early-stage of the diseases.

### OBJECTIVE OF THE INVENTION

One object of the present invention is a method for the treatment of, and delaying the progression of, a neurodegenerative disease (including early-stage of a neurodegenerative disease) in a human subject in need thereof, more specifically for the treatment and/or delaying the progression of Parkinson's disease (such as early Parkinson's disease), Alzheimer's disease and other synucleinopathies associated with c-Abl. Further, it is an object of the present invention to provide a highly effective and safe method for the treatment of a neurodegenerative disease in a human subject, preferably for the treatment of Parkinson's disease, more preferably for the treatment of early-stage Parkinson's disease. Another object of the present invention to provide a method of reducing the rate of progression of early-stage Parkinson's disease. It is another object of the present invention to provide a method for reducing the rate of progression of movement deficits resulting from Parkinson's disease. It is another object of the present invention to provide a method of regulating or preserving autonomic nervous function in a subject (e.g., a human) having early-stage Parkinson's disease. It is another object of the present invention to provide a method of reducing the deterioration of autonomic nervous function in a subject (e.g., a human) having early-stage Parkinson's disease.

Another object of the present invention is a method of treatment or delaying or inhibiting or suppressing the progression of a neurodegenerative disease (e.g., Parkinson's disease) in a subject. It is an object of the present invention to provide a method of treatment or delaying or inhibiting or suppressing the progression of the neurodegenerative disease, wherein the subject is diagnosed at an early-stage of the disease.

Yet another object of the present invention is a method of delaying the progression of Parkinson's disease in a subject. Another particular object of the present invention is a method to reduce the rate of progression of Parkinson's disease in a subject, wherein the subject is diagnosed at an early-stage of the disease.

Yet another object is a method to eliminate symptoms associated with neurodegenerative disease in a subject in need thereof. Particularly, the object is to provide a method to eliminate the symptoms associated with neurodegenerative disease or early-stage neurodegenerative disease in a subject, wherein the subject is in early-stage of the disease.

More particularly, the object is to provide a method to eliminate the symptoms associated with Parkinson's disease in a subject, wherein the subject is in early-stage of the disease.

Yet another object of the present invention is the use of a c-Abl inhibitor for any of the objectives and/or methods described herein.

Yet another object of the present invention is the use of the compound of Formula I;

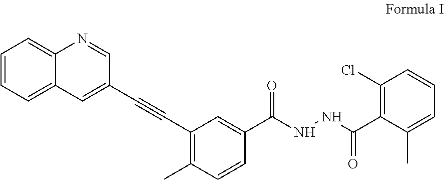 [SMILES: Cc1ccc(C(=O)NNC(=O)c2c(C)cccc2Cl)cc1C#Cc1cnc2ccccc2c1]

or a pharmaceutically acceptable salt thereof for any of the objectives and/or methods described herein.

Another object of the present invention is to provide a c-Abl inhibitor for the treatment, or delay, inhibition, or suppression of the progression of, a neurodegenerative disease in a subject in need thereof, wherein the c-Abl inhibitor is efficacious and safe. Accordingly, it is an object of the present invention to provide a c-Abl inhibitor for the treatment, or delay, inhibition, or suppression of the progression of, a neurodegenerative disease, wherein the c-Abl inhibitor delays the progression of the neurodegenerative disease, reduces the rate of progression of the disease, or eliminates the symptoms associated with the neurodegenerative disease in a subject who is in early-stage of the disease.

Another object of the present invention is a pharmaceutical composition for the treatment, or delaying the progression of, a neurodegenerative disease in a subject in need thereof, wherein the composition is efficacious and safe. Preferably, the composition effectively reduces the rate of progression of the disease or eliminates the symptoms associated with a neurodegenerative disease in a subject who is in the early-stage of the disease.

Yet another object of the present invention is the use of the compound of Formula I or a pharmaceutically acceptable salt thereof for any of the objectives and/or methods described herein.

### SUMMARY OF THE INVENTION

The present inventors have surprisingly found that vodobatinib delays (or reduces the rate of) progression of neurodegenerative diseases, and in particular early-stage Parkinson's disease, in a subject (e.g., a human subject).

One aspect of the present invention is a method for the treatment, or delay, inhibition, or suppression of the progression of, a neurodegenerative disease in a subject (e.g., a human subject) in need thereof, by administering (e.g., an effective amount of) a c-Abl inhibitor to the subject.

Yet another aspect of the present invention is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) a c-Abl inhibitor to the subject, wherein the subject exhibits a disease severity according to modified Hoehn & Yahr stage≤2.

Yet another aspect of the present invention is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) a c-Abl inhibitor to the subject, wherein the subject exhibits a Montreal cognitive assessment score of at least 25.

Yet another aspect of the present invention is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) a c-Abl inhibitor to the subject, wherein the subject exhibits a Dopamine Transporter Single Photon Emission Computed Tomography (DaT SPECT) consistent with the diagnosis of Parkinson's disease.

Yet another aspect of the present invention is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) a c-Abl inhibitor to the subject, wherein the subject is receiving treatment with a monoamine oxidase-B (MAO-B) inhibitor.

Yet another aspect of the present invention is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) a c-Abl inhibitor to the subject, wherein the subject is not concomitantly administering symptomatic medication for Parkinson's disease.

Yet another aspect of the present invention is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) a c-Abl inhibitor to the subject, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of a neurodegenerative disease (such as Parkinson's disease or early-stage Parkinson's disease);
iii) abnormal synuclein deposition as determined by a skin biopsy;
iv) the subject has a body mass index≥18.5 kg/m² and ≤45 kg/m²;
v) an initial diagnosis of a neurodegenerative disease (such as Parkinson's disease or early-stage Parkinson's disease);
vi) the subject is receiving treatment with a monoamine oxidase-B (MAO-B) inhibitor;
vii) the subject is not concomitantly administered symptomatic medication for a neurodegenerative disease (such as Parkinson's disease or early-stage Parkinson's disease);
viii) the subject has not received dopaminergic therapy for at least 30 days prior to the administration;
ix) the subject has been diagnosed with “clinically probable neurodegenerative disease” (such as “clinically probable Parkinson's disease” or “clinically probable early-stage Parkinson's disease”) according to the MDS clinical diagnostic criteria; and
x) the subject exhibits a Montreal cognitive assessment score of at least 25.

Yet another aspect of the present invention is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) a compound of formula I to the subject, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of a neurodegenerative disease (such as Parkinson's disease or early-stage Parkinson's disease);
iii) abnormal synuclein deposition as determined by a skin biopsy;
iv) the subject has a body mass index≥18.5 kg/m² and ≤45 kg/m²;
v) an initial diagnosis of a neurodegenerative disease (such as Parkinson's disease or early-stage Parkinson's disease);
vi) the subject is receiving treatment with a monoamine oxidase-B (MAO-B) inhibitor;
vii) the subject is not concomitantly administering symptomatic medication for a neurodegenerative disease (such as Parkinson's disease or early-stage Parkinson's disease);
viii) the subject has not received dopaminergic therapy for at least 30 days prior to the administration;
ix) the subject has been diagnosed with “clinically probable neurodegenerative disease” (such as “clinically probable Parkinson's disease” or “clinically probable early-stage Parkinson's disease”) according to the MDS clinical diagnostic criteria; and
x) the subject exhibits a Montreal cognitive assessment score of at least 25.

Yet another aspect of the present invention is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) a c-Abl inhibitor to the subject, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administered symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

In a preferred embodiment, the c-Abl inhibitor is a compound of Formula I or a pharmaceutically acceptable salt thereof.

For a compound to be effective in the treatment of neurodegenerative disease, it is necessary for it to be present in an effective amount in the brain. There are several c-Abl inhibitors that have been shown to be present in the brain but do not show much effect in the treatment of neurodegenerative disease. For example, nilotinib was not efficacious in Parkinson's disease patients at administered doses (150 and 300 mg) and one of the reasons for its failure was believed to be lack of sufficient CSF exposure [see Simuni et al. JAMA Neurol., 2021; 78(3):312-320]. There were limitations as a higher dosage could cause cardiovascular toxicity.

Surprisingly, the present inventors have found that the compound of Formula I exhibits higher CSF exposures when administered in a therapeutically effective amount as compared to other c-Abl inhibitors.

Furthermore, the present inventors unexpectedly found that the compound of Formula I when administered to the subject in a therapeutically effective amount resulted in a ratio of concentration of compound of Formula I or a pharmaceutically acceptable salt thereof in CSF to the IC₅₀ of c-Abl inhibition of not less than 1. This was particularly surprising because this ratio for nilotinib, when administered at the highest dosage was about 0.2, which is considerably lower than the ratio that resulted from administering the lowest therapeutically effective amount of the compound of Formula I.

Further it was also observed by the present inventors that the compound of Formula I, when administered in a therapeutically effective amount resulted in a plasma concentration that gave CSF exposure which was safe and efficacious for the treatment, or delay, inhibition, or suppression of the progression of, neurodegenerative disease.

Therefore, in an embodiment, the present invention provides a method for the treatment, or delay, inhibition, or suppression of the progression of, a neurodegenerative disease comprising administering (e.g., orally) a therapeutically effective amount of compound of Formula I, or a pharmaceutically acceptable salt thereof, to a subject in need thereof.

Yet another aspect is a method of treatment of a neurodegenerative disease in a subject (e.g. a human) comprising administering to the subject a compound of formula I or a pharmaceutically acceptable salt thereof in an amount sufficient to achieve a mean plasma AUC₀₋₂₄ ranging from about 5000 ng*h/mL to about 75000 ng*h/mL.

Yet another aspect is a method of treatment of a neurodegenerative disease in a subject (e.g. a human) comprising administering to the subject (e.g. a human) a compound of formula I or a pharmaceutically acceptable salt thereof in an amount sufficient to achieve a mean plasma AUC₀₋₂₄ ranging from about 20,000 ng*h/mL to about 60,000 ng*h/mL.

Another aspect is a method of treatment of a neurodegenerative disease in a subject (e.g. a human) comprising administering to the subject a compound of formula I or a pharmaceutically acceptable salt thereof in an amount sufficient to achieve a plasma Cₘₐₓ ranging from about 500 ng/mL to about 15000 ng/mL.

Another aspect is a method of treatment of a neurodegenerative disease in a subject (e.g. a human) comprising administering to the subject a compound of formula I or a pharmaceutically acceptable salt thereof in an amount sufficient to achieve a plasma Cₘₐₓ ranging from about 1800 ng/mL to about 12,000 ng/mL.

Another aspect is a method of treatment of a neurodegenerative disease in a subject (e.g. a human) comprising administering to the subject a compound of formula I or a pharmaceutically acceptable salt thereof in an amount sufficient to achieve a cerebrospinal fluid Cₘₐₓ ranging from about 0.4 ng/mL to about 7.0 ng/mL.

Another aspect is a method of treatment of a neurodegenerative disease in a subject (e.g. a human) comprising administering to the subject a compound of formula I or a pharmaceutically acceptable salt thereof in an amount sufficient to achieve a cerebrospinal fluid Cₘₐₓ ranging from about 2.0 ng/mL to about 7.0 ng/mL.

In one embodiment of any of the methods and uses described herein, wherein the subject (e.g. a human) achieves cerebrospinal fluid Cₘₐₓ no later than 6 hours from the time of administration of a compound of formula I or a pharmaceutically acceptable salt thereof.

Yet another aspect is a method of treatment of a neurodegenerative disease in a subject (e.g. a human) comprising administering to the subject a compound of formula I or a pharmaceutically acceptable salt thereof in an amount sufficient to achieve a cerebrospinal concentration from about 0.2 ng/mL to about 2.7 ng/mL.

Yet another aspect is a method of treatment of a neurodegenerative disease in a subject (e.g. a human) comprising administering to the subject a compound of formula I or a pharmaceutically acceptable salt thereof in an amount sufficient to achieve a cerebrospinal concentration from about 0.6 ng/mL to about 2.7 ng/mL.

Another aspect is a method of treatment of a neurodegenerative disease in a subject (e.g. a human) comprising administering to the subject a compound of formula I or a pharmaceutically acceptable salt thereof in an amount sufficient to achieve a mean cerebrospinal C_{avg} from about 0.3 ng/mL to about 4.0 ng/mL.

Another aspect is a method of treatment of a neurodegenerative disease in a subject (e.g. a human) comprising administering to the subject a compound of formula I or a pharmaceutically acceptable salt thereof in an amount sufficient to achieve a mean cerebrospinal C_{avg} from about 2.0 ng/mL to about 3.0 ng/mL.

Yet another aspect is a method of treatment of a neurodegenerative disease in a subject (e.g. a human) comprising administering to the subject a compound of formula I or a pharmaceutically acceptable salt thereof in an amount sufficient to achieve a ratio of cerebrospinal Cₘₐₓ to IC₅₀ of cAbl inhibition of not less than 1.

Yet another aspect is a method of treatment of a neurodegenerative disease in a subject (e.g. a human) comprising administering to the subject a compound of formula I or a pharmaceutically acceptable salt thereof in an amount sufficient to achieve a ratio of mean cerebrospinal Cₘₐₓ to IC₅₀ of cAbl inhibition from 1 to 14.

Another aspect is a method of treatment of a neurodegenerative disease in a subject (e.g. a human) comprising administering to the subject a compound of formula I or a pharmaceutically acceptable salt thereof in an amount sufficient to achieve a ratio of Cₘₐₓ to IC₅₀ greater than 5.

Another aspect is a method of treatment of a neurodegenerative disease in a subject (e.g. a human) comprising administering to the subject a compound of formula I or a pharmaceutically acceptable salt thereof in an amount sufficient to cross the blood-brain barrier.

In one embodiment of any of the methods and uses described herein, the neurodegenerative disease includes but not limited to Parkinson's disease (PD), Alzheimer's disease (AD), synucleinopathies, Lewy body dementia (LBD), rapid eye movement (REM), sleep behaviour disorder, multiple system atrophy (MSA), early-stage Parkinson's disease, early stage Alzheimer's disease, early stage of synucleinopathies, early-stage lewy body dementia, early-stage rapid eye movement, early-stage sleep behaviour disorder and early-stage multiple system atrophy.

In one embodiment of any of the methods and uses described herein, the subject (e.g. a human) has had an initial diagnosis of a neurodegenerative disease. In a preferred embodiment, the subject (e.g. a human) has had an initial diagnosis of a Parkinson' disease.

In one embodiment of any of the methods and uses described herein, the subject (e.g. a human) has had an initial diagnosis of a neurodegenerative disease within 3 years of initiating treatment with a compound of formula I or a pharmaceutically acceptable salt thereof.

In one embodiment of any of the methods and uses described herein, the administration of a compound of formula I or a pharmaceutically acceptable salt thereof reduces the progression of a neurodegenerative disease in the subject (e.g. a human).

In one embodiment of any of the methods and uses described herein, the reduction in progression of the neurodegenerative disease is achieved by increasing the time of advancement of the subject (e.g. a human) from part 1 to 2 score to part 2 to 3 score as measured on the Movement Disorder Society-Unified Parkinson's Disease Rating Scale (MDS-UPDRS).

Yet another embodiment is a method for reducing the rate of progression of a neurodegenerative disease or reducing the worsening of symptoms of a neurodegenerative disease comprising administering an effective amount of a compound of formula I or a pharmaceutically acceptable salt thereof, wherein the reduction is achieved by increasing the time of advancement of the subject (e.g. a human) from part 1 to 2 score to part 2 to 3 score as measured on the Movement Disorder Society-Unified Parkinson's Disease Rating Scale (MDS-UPDRS). In a preferred embodiment, the time of advancement of the subject (e.g. a human) from part 1 to 2 score is increased by at least 40 weeks. In another embodiment, the time of advancement of the subject (e.g. a human) from part 2 to 3 score is increased by at least 40 weeks.

In one embodiment of any of the methods and uses described herein, the time of advancement of the subject (e.g. a human) from part 1 to 2 score to part 2 to 3 score is increased by at least 40 weeks.

In one embodiment of any of the methods and uses described herein, the administration of a compound of formula I or a pharmaceutically acceptable salt thereof reduces the rate of progression of a neurodegenerative disease. In another embodiment of any of the methods and uses described herein, the administration of a compound of formula I or a pharmaceutically acceptable salt thereof reduces the rate of progression of movement deficits resulting from a neurodegenerative disease in a subject (e.g., a human). Yet another embodiment of any of the methods and uses described herein, the administration of a compound of formula I or a pharmaceutically acceptable salt thereof regulates or preserve autonomic nervous function in a human subject having a neurodegenerative disease.

In one embodiment of any of the methods and uses described herein, the subject (e.g. a human) has a neurodegenerative disease and a disease severity according to modified Hoehn & Yahr stage≤2. In one embodiment of any of the methods and uses described herein, the subject (e.g. a human) has been receiving treatment with a monoamine oxidase B (MAOB) inhibitor. In one embodiment of any of the methods and uses described herein, the subject (e.g. a human) is not being treated with any dopamine treatment other than a monoamine oxidase-B (MAO-B) inhibitor. In another embodiment of any of the methods and uses described herein, the subject (e.g. a human) has not received dopaminergic therapy for at least 30 days prior to the administration. Yet another embodiment of any of the methods and uses described herein, the subject (e.g. a human) exhibits a Montreal cognitive assessment score of at least 25. In one embodiment of any of the methods and uses described herein, the subject (e.g. a human) is not concomitantly administering symptomatic medication for Parkinson's disease. In one embodiment of any of the methods and uses described herein, the subject (e.g. a human) has been diagnosed with “clinically probable Parkinson's disease” according to the MDS clinical diagnostic criteria. In another embodiment of any of the methods and uses described herein, the subject (e.g. a human) has a Dopamine Transporter Single Photon Emission Computed Tomography (DaT SPECT) consistent with the diagnosis of Parkinson's disease. In one embodiment of any of the methods and uses described herein, the subject (e.g. a human) exhibits abnormal synuclein deposition as determined by a skin punch biopsy. In one embodiment of any of the methods and uses described herein, the neurodegenerative disease (e.g. Parkinson's disease) is an early-stage neurodegenerative disease (e.g., early-stage Parkinson's disease).

In one embodiment of any of the methods and uses described herein, the subject (e.g. a human) has had an initial diagnosis of Parkinson's disease.

In one embodiment of any of the methods and uses described herein, the subject (e.g. a human) has had an initial diagnosis of Parkinson's disease within 3 years of initiating treatment with a compound of formula I or a pharmaceutically acceptable salt thereof.

In one embodiment of any of the methods and uses described herein, the administration of a compound of formula I or a pharmaceutically acceptable salt thereof reduces the progression of Parkinson's disease in the subject (e.g. a human).

In one embodiment of any of the methods and uses described herein, the reduction in progression of Parkinson's disease is achieved by increasing the time of advancement of the subject (e.g. a human) from part 1 to 2 score to part 2 to 3 score as measured on the Movement Disorder Society-Unified Parkinson's Disease Rating Scale (MDS-UPDRS).

Yet another aspect is a method for reducing the rate of progression of Parkinson's disease or reducing the worsening of symptoms of Parkinson's disease comprising administering an effective amount of a compound of formula I or a pharmaceutically acceptable salt thereof, wherein the reduction is achieved by increasing the time of advancement of the subject (e.g. a human) from part 1 to 2 score to part 2 to 3 score as measured on the Movement Disorder Society-Unified Parkinson's Disease Rating Scale (MDS-UPDRS). In a preferred embodiment, the time of advancement of the subject (e.g. a human) from part 1 to 2 score is increased by at least 40 weeks. In another embodiment, the time of advancement of the subject (e.g. a human) from part 2 to 3 score is increased by at least 40 weeks.

In one embodiment of any of the methods and uses described herein, the time of advancement of the subject (e.g. a human) from part 1 to 2 score to part 2 to 3 score is increased by at least 40 weeks.

In one embodiment of any of the methods and uses described herein, the Parkinson's disease is early-stage Parkinson's disease. In one embodiment of any of the methods and uses described herein, the administration of a compound of formula I or a pharmaceutically acceptable salt thereof reduces the rate of progression of early-stage Parkinson's disease. In another embodiment of any of the methods and uses described herein, the administration of a compound of formula I or a pharmaceutically acceptable salt thereof reduces the rate of progression of movement deficits resulting from Parkinson's disease in a human subject suffering from early-stage Parkinson's disease. Yet another embodiment of any of the methods and uses described herein, the administration of a compound of formula I or a pharmaceutically acceptable salt thereof regulates or preserve autonomic nervous function in a human subject having early-stage Parkinson's disease. In another embodiment of any of the methods and uses described herein, the administration of a compound of formula I or a pharmaceutically acceptable salt thereof reduces the rate of the deterioration of autonomic nervous function due to early-stage Parkinson's disease.

In one embodiment of any of the methods and uses described herein, the subject (e.g. a human) has early-stage Parkinson's disease and a disease severity according to modified Hoehn & Yahr stage≤2.

In one embodiment of any of the methods and uses described herein, the subject (e.g. a human) has been receiving treatment with a monoamine oxidase B (MAOB) inhibitor. In one embodiment of any of the methods and uses described herein, the subject (e.g. a human) is not being treated with any dopamine treatment other than a monoamine oxidase-B (MAO-B) inhibitor. In another embodiment of any of the methods and uses described herein, the subject (e.g. a human) has not received dopaminergic therapy for at least 30 days prior to the administration. Yet another embodiment of any of the methods and uses described herein, the subject (e.g. a human) exhibits a Montreal cognitive assessment score of at least 25. In one embodiment of any of the methods and uses described herein, the subject (e.g. a human) is not concomitantly administering symptomatic medication for Parkinson's disease. In one embodiment of any of the methods and uses described herein, the subject (e.g. a human) has been diagnosed with “clinically probable Parkinson's disease” according to the MDS clinical diagnostic criteria. In another embodiment of any of the methods and uses described herein, the subject (e.g. a human) has a Dopamine Transporter Single Photon Emission Computed Tomography (DaT SPECT) consistent with the diagnosis of Parkinson's disease. In one embodiment of any of the methods and uses described herein, the subject (e.g. a human) exhibits abnormal synuclein deposition as determined by a skin punch biopsy.

In one embodiment of any of the methods and uses described herein, the administration of a compound of formula I or a pharmaceutically acceptable salt thereof increases the time to significant worsening of the subject (e.g. a human) on the Movement Disorder Society-Unified Parkinson's Disease Rating Scale (MDS-UPDRS) Parts II and III.

Yet another embodiment of any of the methods and uses described herein, the administration of a compound of formula I or a pharmaceutically acceptable salt thereof increases the time to significant worsening of the subject (e.g. a human) on the Movement Disorder Society-Unified Parkinson's Disease Rating Scale (MDS-UPDRS) Parts I, II and III.

Yet another embodiment of any of the methods and uses described herein, the administration of a compound of formula I or a pharmaceutically acceptable salt thereof slows progression of overall severity of the subject (e.g. a human) Parkinson's disease as measured by the Clinical Global Impression Severity (CGIS) scale.

In one embodiment of any of the methods and uses described herein, the subject (e.g. a human) is administered the compound of formula I or a pharmaceutically acceptable salt thereof in a fasting state. In one embodiment of any of the methods and uses described herein, the subject (e.g. a human) is administered the compound of formula I or a pharmaceutically acceptable salt thereof in a fasting state at least 1 hour prior to administration. In another embodiment of any of the methods and uses described herein, the subject (e.g. a human) is administered the compound of formula I or a pharmaceutically acceptable salt thereof in a fasting state at least 2 hours prior to administration.

In one embodiment of any of the methods and uses described herein, the subject (e.g. a human) is administered the compound of formula I or a pharmaceutically acceptable salt thereof in a fed state. In one embodiment of any of the methods and uses described herein, the subject (e.g. a human) is administered the compound of formula I or a pharmaceutically acceptable salt thereof shortly after completion of the meal, preferably after 10 minutes, more preferably after 30 minutes.

In one embodiment of any of the methods and uses described herein, the subject (e.g. a human) is aged 50 years or older. In another embodiment of any of the methods and uses described herein, the subject (e.g. a human) is aged 20 years or older.

In one embodiment of any of the methods and uses described herein, the subject (e.g. a human) is not concomitantly receiving dopamine replacement medication.

In another embodiment of any of the methods and uses described herein, the subject (e.g., human) has a body mass index≥18.5 kg/m² and ≤45 kg/m².

In one embodiment of any of the methods and uses described herein, the subject (e.g. a human) is administered about 5 mg to about 480 mg of a compound of formula I or a pharmaceutically acceptable salt thereof. In one embodiment of any of the methods and uses described herein, the subject (e.g. a human) is administered about 48 mg to about 480 mg of a compound of formula I or a pharmaceutically acceptable salt thereof. In another embodiment, the subject (e.g. a human) is administered about 48 mg or 96 mg or 144 mg or 192 mg or 240 mg or 288 mg or 336 mg or 384 mg or 432 mg or 480 mg. In a preferred embodiment, the subject (e.g. a human) is administered about 134 mg to about 384 mg of a compound of formula I or a pharmaceutically acceptable salt thereof. In a more preferred embodiment, the subject (e.g. a human) is administered about 192 mg or about 384 mg of a compound of formula I or a pharmaceutically acceptable salt thereof.

In one embodiment of any of the methods and uses described herein the subject (e.g. a human) is administered about 192 mg or about 384 mg of a compound of formula I or a pharmaceutically acceptable salt thereof in the form of a solid dosage form.

In one embodiment of any of the methods and uses described herein, upon the occurrence of an adverse event, the daily dose of a compound of formula I or a pharmaceutically acceptable salt thereof is reduced by half.

In one embodiment of any of the methods and uses described herein, the compound of formula I or a pharmaceutically acceptable salt thereof is administered orally.

In one embodiment of any of the methods and uses described herein, the compound of formula I or a pharmaceutically acceptable salt thereof is administered once a day.

In one embodiment of any of the methods and uses described herein, the compound of formula I or a pharmaceutically acceptable salt thereof is administered twice a day.

In one embodiment of any of the methods and uses described herein, the subject (e.g. a human) (i) is at least 50 years of age, (ii) has had an initial diagnosis of Parkinson's disease within 3 years of initiating treatment with a compound of formula I or a pharmaceutically acceptable salt thereof, (iii) has a score on a modified Hoehn and Yahr stage of 2 or less, and (iv) is not being treated with any dopamine treatment other than a monoamine oxidase-B (MAO-B) inhibitor. Yet another aspect is a method for the treatment or delay, inhibition, or suppression of the progression of, a neurodegenerative disease (such as Parkinson's disease) in a subject (e.g., a human subject) in need thereof, wherein the method comprises administering (e.g., an effective amount of) to the subject a compound of Formula I or a pharmaceutically acceptable salt thereof.

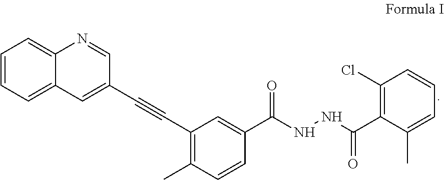 [SMILES: Cc1ccc(C(=O)NNC(=O)c2c(C)cccc2Cl)cc1C#Cc1cnc2ccccc2c1]

Yet another aspect is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) to the subject a compound of Formula I or a pharmaceutically acceptable salt thereof.

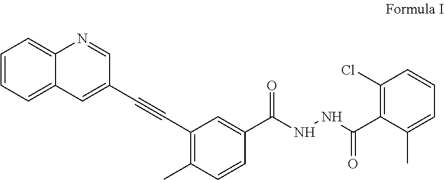 [SMILES: Cc1ccc(C(=O)NNC(=O)c2c(C)cccc2Cl)cc1C#Cc1cnc2ccccc2c1]

wherein the subject exhibits a disease severity according to modified Hoehn & Yahr stage≤2.

Yet another aspect is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) to the subject a compound of Formula I or a pharmaceutically acceptable salt thereof:

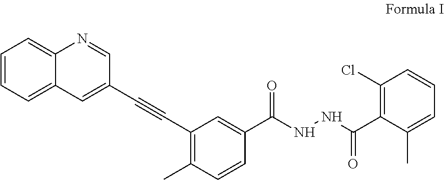 [SMILES: Cc1ccc(C(=O)NNC(=O)c2c(C)cccc2Cl)cc1C#Cc1cnc2ccccc2c1]

wherein the subject exhibits a Montreal cognitive assessment score of at least 25.

Yet another aspect is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) to the subject a compound of Formula I or a pharmaceutically acceptable salt thereof:

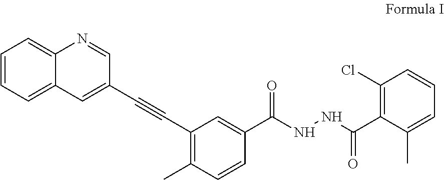 [SMILES: Cc1ccc(C(=O)NNC(=O)c2c(C)cccc2Cl)cc1C#Cc1cnc2ccccc2c1]

wherein the subject exhibits a Dopamine Transporter Single Photon Emission Computed Tomography (DaT SPECT) consistent with the diagnosis of Parkinson's disease.

Yet another aspect is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) to the subject a compound of Formula I or a pharmaceutically acceptable salt thereof:

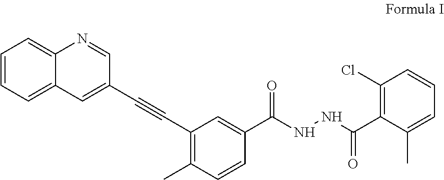 [SMILES: Cc1ccc(C(=O)NNC(=O)c2c(C)cccc2Cl)cc1C#Cc1cnc2ccccc2c1]

wherein the subject is receiving treatment with a monoamine oxidase-B (MAO-B) inhibitor.

Yet another aspect is a method for the treatment, or delay, inhibition, or suppression of the progression of, a neurodegenerative disease (such as Parkinson's disease) in a subject (e.g., a human subject) in need thereof, wherein the method comprises administering (e.g., an effective amount of) to the subject a compound of Formula I or a pharmaceutically acceptable salt thereof:

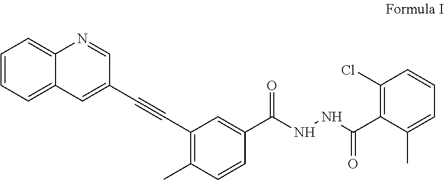 [SMILES: Cc1ccc(C(=O)NNC(=O)c2c(C)cccc2Cl)cc1C#Cc1cnc2ccccc2c1]

wherein the subject is not concomitantly administering symptomatic medication for Parkinson's disease.

Yet another aspect is a method for the treatment, or delay, inhibition, or suppression of the progression of, a neurodegenerative disease (such as Parkinson's disease or early-stage Parkinson's disease), wherein the method comprises administering (e.g., orally) a compound of Formula I or a pharmaceutically acceptable salt thereof:

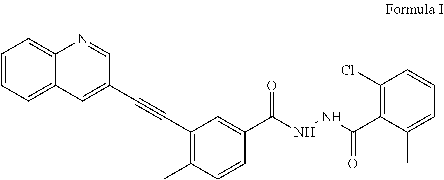 [SMILES: Cc1ccc(C(=O)NNC(=O)c2c(C)cccc2Cl)cc1C#Cc1cnc2ccccc2c1]

to a subject (e.g., a human) in need thereof in an amount sufficient to achieve a mean plasma AUC₀₋₂₄ ranging from about 5,000 ng*h/mL to about 75,000 ng*h/mL, preferably from about 20,000 ng*h/mL to about 60,000 ng*h/mL, more preferably from about 15,000 ng*h/mL to about 55,000 ng*h/mL.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

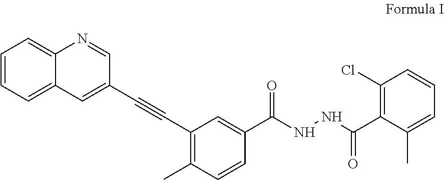 [SMILES: Cc1ccc(C(=O)NNC(=O)c2c(C)cccc2Cl)cc1C#Cc1cnc2ccccc2c1]

to a subject (e.g., a human) in need thereof in an amount sufficient to achieve a mean plasma AUC₀₋₂₄ ranging from about 5,000 ng*h/mL to about 75,000 ng*h/mL, wherein the subject exhibits a disease severity according to modified Hoehn & Yahr stage≤2.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

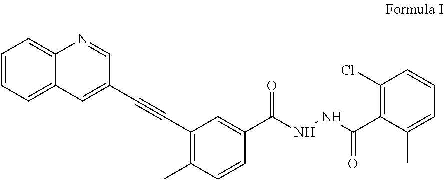 [SMILES: Cc1ccc(C(=O)NNC(=O)c2c(C)cccc2Cl)cc1C#Cc1cnc2ccccc2c1]

to a subject (e.g., a human) in need thereof in an amount sufficient to achieve a mean plasma AUC₀₋₂₄ ranging from about 5,000 ng*h/mL to about 75,000 ng*h/mL, wherein the subject exhibits a Montreal cognitive assessment score of at least 25.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

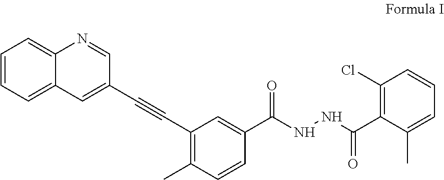 [SMILES: Cc1ccc(C(=O)NNC(=O)c2c(C)cccc2Cl)cc1C#Cc1cnc2ccccc2c1]

to a subject in need thereof in an amount sufficient to achieve a mean plasma AUC₀₋₂₄ ranging from about 5,000 ng*h/mL to about 75,000 ng*h/mL, wherein the subject exhibits a Dopamine Transporter Single Photon Emission Computed Tomography (DaT SPECT) consistent with the diagnosis of Parkinson's disease.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

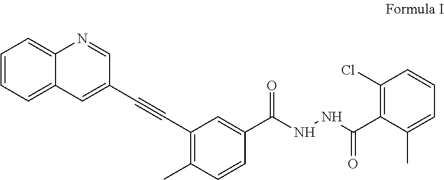 [SMILES: Cc1ccc(C(=O)NNC(=O)c2c(C)cccc2Cl)cc1C#Cc1cnc2ccccc2c1]

to a subject in need thereof in an amount sufficient to achieve a mean plasma AUC₀₋₂₄ ranging from about 5,000 ng*h/mL to about 75,000 ng*h/mL, wherein the subject is receiving treatment with a monoamine oxidase-B (MAO-B) inhibitor.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

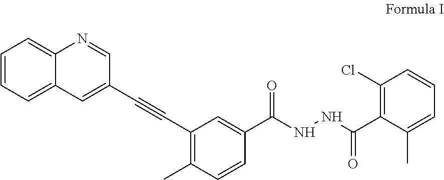 [SMILES: Cc1ccc(C(=O)NNC(=O)c2c(C)cccc2Cl)cc1C#Cc1cnc2ccccc2c1]

to a subject in need thereof in an amount sufficient to achieve a mean plasma AUC₀₋₂₄ ranging from about 5,000 ng*h/mL to about 75,000 ng*h/mL, wherein the subject is not concomitantly administering symptomatic medication for Parkinson's disease.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

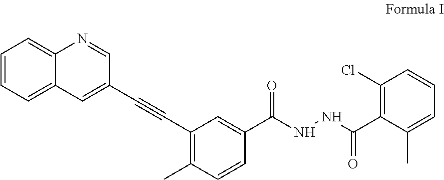 [SMILES: Cc1ccc(C(=O)NNC(=O)c2c(C)cccc2Cl)cc1C#Cc1cnc2ccccc2c1]

to a human subject in need thereof in an amount sufficient to achieve a mean plasma AUC₀₋₂₄ ranging from about 5,000 ng*h/mL to about 75,000 ng*h/mL, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

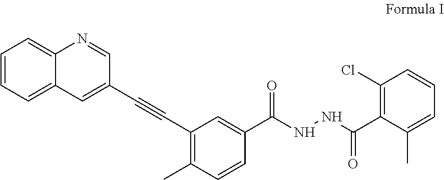 [SMILES: Cc1ccc(C(=O)NNC(=O)c2c(C)cccc2Cl)cc1C#Cc1cnc2ccccc2c1]

to a subject in need thereof in an amount sufficient to achieve a plasma Cₘₐₓ ranging from about 100 ng/mL to about 15,000 ng/mL, preferably plasma Cₘₐₓ ranging from about 500 ng/mL to about 15,000 ng/mL, more preferably plasma Cₘₐₓ ranging from about 1800 ng/mL to about 12,000 ng/mL.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

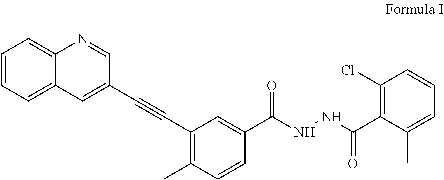 [SMILES: Cc1ccc(C(=O)NNC(=O)c2c(C)cccc2Cl)cc1C#Cc1cnc2ccccc2c1]

to a subject in need thereof in an amount sufficient to achieve a plasma Cₘₐₓ ranging from about 500 ng/mL to about 15,000 ng/mL.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

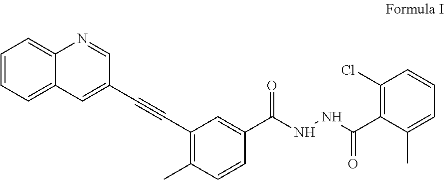 [SMILES: Cc1ccc(C(=O)NNC(=O)c2c(C)cccc2Cl)cc1C#Cc1cnc2ccccc2c1]

to a subject in need thereof in an amount sufficient to achieve a plasma Cₘₐₓ ranging from about 500 ng/mL to about 15,000 ng/mL, wherein the subject exhibits a disease severity according to modified Hoehn & Yahr stage≤2.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

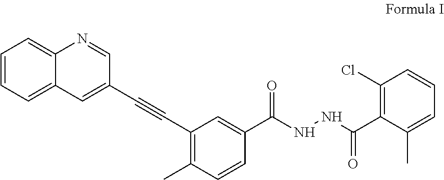 [SMILES: Cc1ccc(C(=O)NNC(=O)c2c(C)cccc2Cl)cc1C#Cc1cnc2ccccc2c1]

to a subject in need thereof in an amount sufficient to achieve a plasma Cₘₐₓ ranging from about 500 ng/mL to about 15,000 ng/mL, wherein the subject exhibits a Montreal cognitive assessment score of at least 25.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

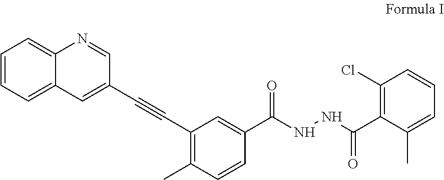 [SMILES: Cc1ccc(C(=O)NNC(=O)c2c(C)cccc2Cl)cc1C#Cc1cnc2ccccc2c1]

to a subject in need thereof in an amount sufficient to achieve a plasma Cₘₐₓ ranging from about 500 ng/mL to about 15,000 ng/mL, wherein the subject exhibits a Dopamine Transporter Single Photon Emission Computed Tomography (DaT SPECT) consistent with the diagnosis of Parkinson's disease.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

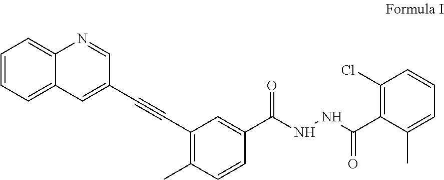 [SMILES: Cc1ccc(C(=O)NNC(=O)c2c(C)cccc2Cl)cc1C#Cc1cnc2ccccc2c1]

to a subject in need thereof in an amount sufficient to achieve a plasma Cₘₐₓ ranging from about 500 ng/mL to about 15,000 ng/mL, wherein the subject is receiving treatment with a monoamine oxidase-B (MAO-B) inhibitor.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

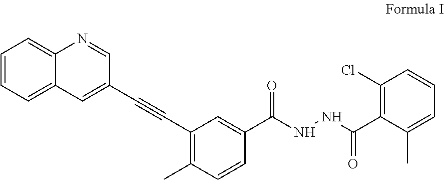 [SMILES: Cc1ccc(C(=O)NNC(=O)c2c(C)cccc2Cl)cc1C#Cc1cnc2ccccc2c1]

to a subject in need thereof in an amount sufficient to achieve a plasma Cₘₐₓ ranging from about 500 ng/mL to about 15,000 ng/mL, wherein the subject is not concomitantly administering symptomatic medication for Parkinson's disease.

Yet another aspect is a method for the treatment, or delay, inhibition, or suppression of the progression of, a neurodegenerative disease (such as Parkinson's disease or early-stage Parkinson' disease), wherein the method comprises administering (e.g., orally) a compound of Formula I or a pharmaceutically acceptable salt thereof,

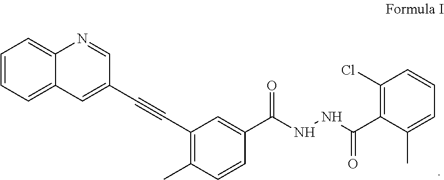 [SMILES: Cc1ccc(C(=O)NNC(=O)c2c(C)cccc2Cl)cc1C#Cc1cnc2ccccc2c1]

to a subject in need thereof in an amount sufficient to achieve a plasma Cₘₐₓ ranging from about 500 ng/mL to about 15,000 ng/mL, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Yet another aspect is a method for the treatment, or delay, inhibition, or suppression of the progression of, a neurodegenerative disease (e.g., Parkinson's disease), wherein the method comprises administering (e.g., orally) a compound of Formula I or a pharmaceutically acceptable salt thereof,

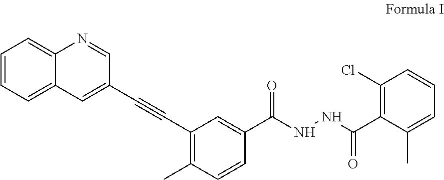 [SMILES: Cc1ccc(C(=O)NNC(=O)c2c(C)cccc2Cl)cc1C#Cc1cnc2ccccc2c1]

to a subject in need thereof in an amount sufficient to achieve a CSF Cₘₐₓ ranging from about 0.4 ng/mL to about 7.0 ng/mL, preferably from about 2 ng/mL to about 7.0 ng/mL. In a preferred embodiment, the subject achieves cerebrospinal fluid Cₘₐₓ no later than 6 hours from the time of administration of a compound of Formula I or a pharmaceutically acceptable salt thereof.

Yet another aspect is a method for the treatment, or delay, inhibition, or suppression of the progression of, a neurodegenerative disease (e.g., Parkinson's disease), wherein the method comprises administering (e.g., orally) a compound of Formula I or a pharmaceutically acceptable salt thereof,

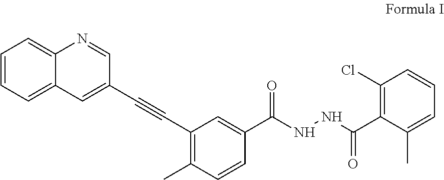 [SMILES: Cc1ccc(C(=O)NNC(=O)c2c(C)cccc2Cl)cc1C#Cc1cnc2ccccc2c1]

to a subject in need thereof in an amount sufficient to achieve a CSF Cₘₐₓ ranging from about 0.4 ng/mL to about 7.0 ng/mL, wherein the subject exhibits a disease severity according to modified Hoehn & Yahr stage≤2. In a preferred embodiment, the subject achieves cerebrospinal fluid Cₘₐₓ no later than 6 hours from the time of administration of a compound of Formula I or a pharmaceutically acceptable salt thereof.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

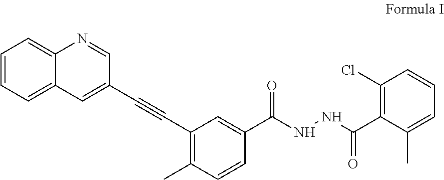 [SMILES: Cc1ccc(C(=O)NNC(=O)c2c(C)cccc2Cl)cc1C#Cc1cnc2ccccc2c1]

to a subject in need thereof in an amount sufficient to achieve a CSF Cₘₐₓ ranging from about 0.4 ng/mL to about 7.0 ng/mL, wherein the subject exhibits a Montreal cognitive assessment score of at least 25. In a preferred embodiment, the subject achieves cerebrospinal fluid Cₘₐₓ no later than 6 hours from the time of administration of a compound of Formula I or a pharmaceutically acceptable salt thereof.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

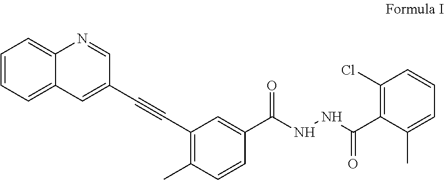 [SMILES: Cc1ccc(C(=O)NNC(=O)c2c(C)cccc2Cl)cc1C#Cc1cnc2ccccc2c1]

to a subject in need thereof in an amount sufficient to achieve a CSF Cₘₐₓ ranging from about 0.4 ng/mL to about 7.0 ng/mL, wherein the subject exhibits a Dopamine Transporter Single Photon Emission Computed Tomography (DaT SPECT) consistent with the diagnosis of Parkinson's disease. In a preferred embodiment, the subject achieves cerebrospinal fluid Cₘₐₓ no later than 6 hours from the time of administration of a compound of Formula I or a pharmaceutically acceptable salt thereof.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

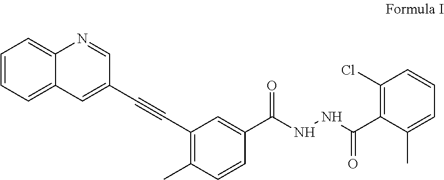 [SMILES: Cc1ccc(C(=O)NNC(=O)c2c(C)cccc2Cl)cc1C#Cc1cnc2ccccc2c1]

to a subject in need thereof in an amount sufficient to achieve a CSF Cₘₐₓ ranging from about 0.4 ng/mL to about 7.0 ng/mL, wherein the subject is receiving treatment with a monoamine oxidase-B (MAO-B) inhibitor. In a preferred embodiment, the subject achieves cerebrospinal fluid Cₘₐₓ no later than 6 hours from the time of administration of a compound of Formula I or a pharmaceutically acceptable salt thereof.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

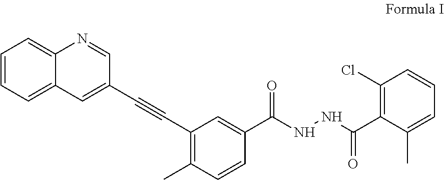 [SMILES: Cc1ccc(C(=O)NNC(=O)c2c(C)cccc2Cl)cc1C#Cc1cnc2ccccc2c1]

to a subject in need thereof in an amount sufficient to achieve a CSF Cₘₐₓ ranging from about 0.4 ng/mL to about 7.0 ng/mL, wherein the subject is not concomitantly administering symptomatic medication for Parkinson's disease. In a preferred embodiment, the subject achieves cerebrospinal fluid Cₘₐₓ no later than 6 hours from the time of administration of a compound of Formula I or a pharmaceutically acceptable salt thereof.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

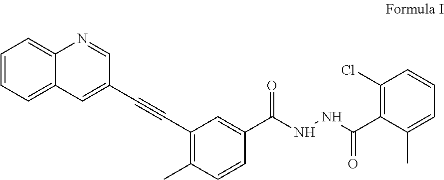 [SMILES: Cc1ccc(C(=O)NNC(=O)c2c(C)cccc2Cl)cc1C#Cc1cnc2ccccc2c1]

to a subject in need thereof in an amount sufficient to achieve a CSF Cₘₐₓ ranging from about 0.4 ng/mL to about 7.0 ng/mL, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

In one embodiment of any of the methods and uses described herein, wherein the subject achieves cerebrospinal fluid Cₘₐₓ no later than 6 hours from the time of administration of a compound of Formula I or a pharmaceutically acceptable salt thereof.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

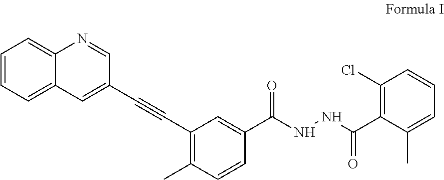 [SMILES: Cc1ccc(C(=O)NNC(=O)c2c(C)cccc2Cl)cc1C#Cc1cnc2ccccc2c1]

to a subject in need thereof in an amount sufficient to achieve a CSF concentration at least about 0.2 ng/mL to about 2.7 ng/mL.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

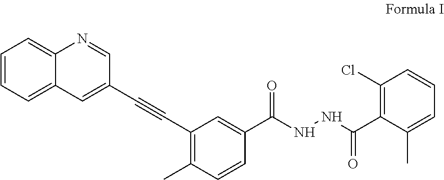 [SMILES: Cc1ccc(C(=O)NNC(=O)c2c(C)cccc2Cl)cc1C#Cc1cnc2ccccc2c1]

to a subject in need thereof in an amount sufficient to achieve a CSF concentration at least about 0.6 ng/mL to about 2.7 ng/mL.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

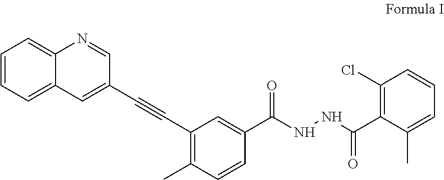 [SMILES: Cc1ccc(C(=O)NNC(=O)c2c(C)cccc2Cl)cc1C#Cc1cnc2ccccc2c1]

to a subject in need thereof in an amount sufficient to achieve a CSF concentration at least about 0.2 ng/mL to about 2.7 ng/mL, wherein the subject exhibits a disease severity according to modified Hoehn & Yahr stage≤2.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

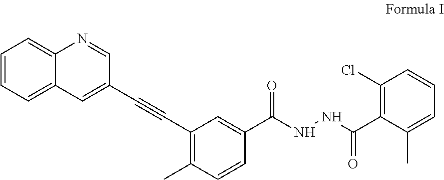 [SMILES: Cc1ccc(C(=O)NNC(=O)c2c(C)cccc2Cl)cc1C#Cc1cnc2ccccc2c1]

to a subject in need thereof in an amount sufficient to achieve a CSF concentration at least about 0.2 ng/mL to about 2.7 ng/mL, wherein the subject exhibits a Montreal cognitive assessment score of at least 25.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

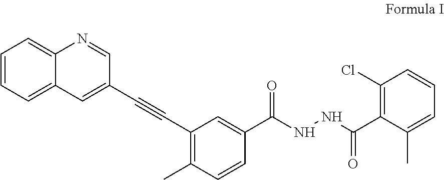 [SMILES: Cc1ccc(C(=O)NNC(=O)c2c(C)cccc2Cl)cc1C#Cc1cnc2ccccc2c1]

to a subject in need thereof in an amount sufficient to achieve a CSF concentration at least about 0.2 ng/mL to about 2.7 ng/mL, wherein the subject exhibits a Dopamine Transporter Single Photon Emission Computed Tomography (DaT SPECT) consistent with the diagnosis of Parkinson's disease.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

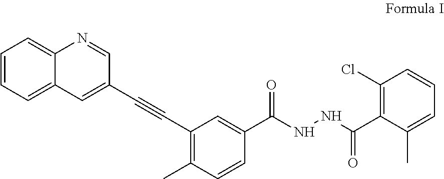 [SMILES: Cc1ccc(C(=O)NNC(=O)c2c(C)cccc2Cl)cc1C#Cc1cnc2ccccc2c1]

to a subject in need thereof in an amount sufficient to achieve a CSF concentration at least about 0.2 ng/mL to about 2.7 ng/mL, wherein the subject is receiving treatment with a monoamine oxidase-B (MAO-B) inhibitor.

Yet another aspect is a method for the treatment, or delay, inhibition, or suppression of the progression of, a neurodegenerative disease (e.g., Parkinson's disease or early-stage Parkinson's disease), wherein the method comprises administering a (e.g., orally) compound of Formula I or a pharmaceutically acceptable salt thereof,

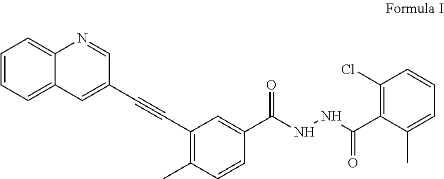 [SMILES: Cc1ccc(C(=O)NNC(=O)c2c(C)cccc2Cl)cc1C#Cc1cnc2ccccc2c1]

to a subject in need thereof in an amount sufficient to achieve a CSF concentration at least about 0.2 ng/mL to about 2.7 ng/mL, wherein the subject is not concomitantly administering symptomatic medication for Parkinson's disease.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

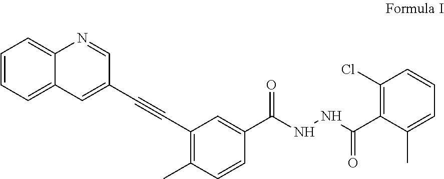 [SMILES: Cc1ccc(C(=O)NNC(=O)c2c(C)cccc2Cl)cc1C#Cc1cnc2ccccc2c1]

to a subject in need thereof in an amount sufficient to achieve a CSF concentration at least about 0.2 ng/mL to about 2.7 ng/mL, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

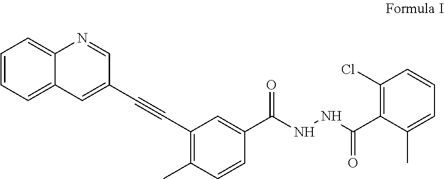 [SMILES: Cc1ccc(C(=O)NNC(=O)c2c(C)cccc2Cl)cc1C#Cc1cnc2ccccc2c1]

to a subject in need thereof in an amount sufficient to achieve a mean CSF C_{avg} of about 0.3 ng/mL to about 4.0 ng/mL.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

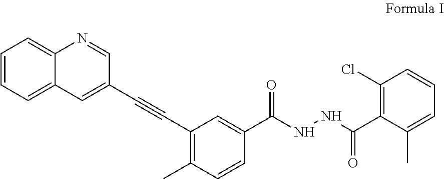 [SMILES: Cc1ccc(C(=O)NNC(=O)c2c(C)cccc2Cl)cc1C#Cc1cnc2ccccc2c1]

to a subject in need thereof in an amount sufficient to achieve a mean CSF C_{avg} of about 2.0 ng/mL to about 3.5 ng/mL.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

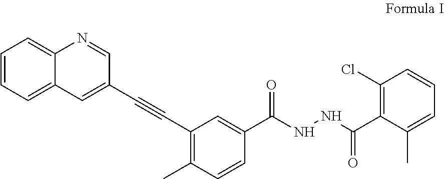 [SMILES: Cc1ccc(C(=O)NNC(=O)c2c(C)cccc2Cl)cc1C#Cc1cnc2ccccc2c1]

to a subject in need thereof in an amount sufficient to achieve a mean CSF C_{avg} of about 0.3 ng/mL to about 4.0 ng/mL, wherein the subject exhibits a disease severity according to modified Hoehn & Yahr stage≤2.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

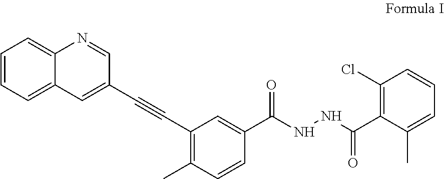 [SMILES: Cc1ccc(C(=O)NNC(=O)c2c(C)cccc2Cl)cc1C#Cc1cnc2ccccc2c1]

to a subject in need thereof in an amount sufficient to achieve a mean CSF C_{avg} of about 0.3 ng/mL to about 4.0 ng/mL, wherein the subject exhibits a Montreal cognitive assessment score of at least 25.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

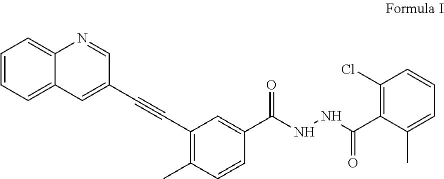 [SMILES: Cc1ccc(C(=O)NNC(=O)c2c(C)cccc2Cl)cc1C#Cc1cnc2ccccc2c1]

to a subject in need thereof in an amount sufficient to achieve a mean CSF C_{avg} of about 0.3 ng/mL to about 4.0 ng/mL, wherein the subject exhibits a Dopamine Transporter Single Photon Emission Computed Tomography (DaT SPECT) consistent with the diagnosis of Parkinson's disease.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

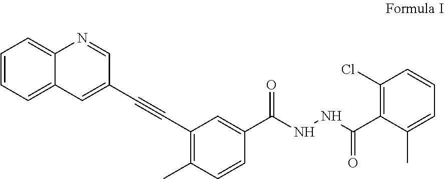 [SMILES: Cc1ccc(C(=O)NNC(=O)c2c(C)cccc2Cl)cc1C#Cc1cnc2ccccc2c1]

to a subject in need thereof in an amount sufficient to achieve a mean CSF C_{avg} of about 0.3 ng/mL to about 4.0 ng/mL, wherein the subject is receiving treatment with a monoamine oxidase-B (MAO-B) inhibitor.

Yet another aspect is a method for the treatment, or delay, inhibition, or suppression of the progression of, a neurodegenerative disease (e.g., Parkinson's disease or early-stage Parkinson's disease), wherein the method comprises administering (e.g., orally) a compound of Formula I or a pharmaceutically acceptable salt thereof,

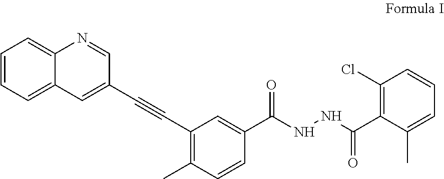 [SMILES: Cc1ccc(C(=O)NNC(=O)c2c(C)cccc2Cl)cc1C#Cc1cnc2ccccc2c1]

to a subject in need thereof in an amount sufficient to achieve a mean CSF C_{avg} of about 0.3 ng/mL to about 4.0 ng/mL, wherein the subject is not concomitantly administering symptomatic medication for Parkinson's disease.

Another aspect is a method for the treatment, or delay, inhibition, or suppression of the progression of, a neurodegenerative disease (e.g., Parkinson's disease), wherein the method comprises administering (e.g., orally) a compound of Formula I or a pharmaceutically acceptable salt thereof,

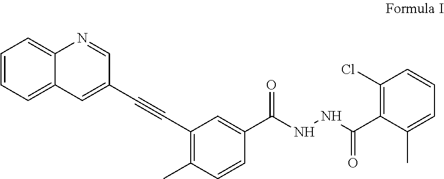 [SMILES: Cc1ccc(C(=O)NNC(=O)c2c(C)cccc2Cl)cc1C#Cc1cnc2ccccc2c1]

to a subject in need thereof in an amount sufficient to achieve a mean CSF C_{avg} of about 0.3 ng/mL to about 4.0 ng/mL, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect of the present invention is a method for the treatment, or delay, inhibition, or suppression of the progression of, a neurodegenerative disease, wherein the method comprises administering a (e.g., orally) c-Abl inhibitor (such as vodobatinib or a pharmaceutically acceptable salt thereof) to a subject (e.g., a human subject) in need thereof in an amount sufficient to achieve a ratio of mean CSF Cₘₐₓ to IC₅₀ of c-Abl inhibition of not less than 1.

Another aspect of the present invention is a method for the treatment, or delay, inhibition, or suppression of the progression of, a neurodegenerative disease, wherein the method comprises administering (e.g., orally) a c-Abl inhibitor (vodobatinib or a pharmaceutically acceptable salt thereof) to a subject (e.g., a human subject) in need thereof in an amount sufficient to achieve a ratio of mean CSF Cₘₐₓ to IC₅₀ of c-Abl inhibition of not less than 1, wherein the subject exhibits a disease severity according to modified Hoehn & Yahr stage≤2.

Another aspect of the present invention is a method for the treatment, or delay, inhibition, or suppression of the progression of, a neurodegenerative disease, wherein the method comprises administering (e.g., orally) a c-Abl inhibitor (vodobatinib or a pharmaceutically acceptable salt thereof) to a subject (e.g., a human subject) in need thereof in an amount sufficient to achieve a ratio of mean CSF Cₘₐₓ to IC₅₀ of c-Abl inhibition of not less than 1, wherein the subject exhibits a Montreal cognitive assessment score of at least 25.

Another aspect of the present invention is a method for the treatment, or delay, inhibition, or suppression of the progression of, a neurodegenerative disease, wherein the method comprises administering (e.g., orally) a c-Abl inhibitor (vodobatinib or a pharmaceutically acceptable salt thereof) to a subject (e.g., a human subject) in need thereof in an amount sufficient to achieve a ratio of mean CSF Cₘₐₓ to IC₅₀ of c-Abl inhibition of not less than 1, wherein the subject exhibits a Dopamine Transporter Single Photon Emission Computed Tomography (DaT SPECT) consistent with the diagnosis of Parkinson's disease.

Another aspect of the present invention is a method for the treatment, or delay, inhibition, or suppression of the progression of, a neurodegenerative disease, wherein the method comprises administering (e.g., orally) a c-Abl inhibitor (vodobatinib or a pharmaceutically acceptable salt thereof) to a subject (e.g., a human subject) in need thereof in an amount sufficient to achieve a ratio of mean CSF Cₘₐₓ to IC₅₀ of c-Abl inhibition of not less than 1, wherein the subject is receiving treatment with a monoamine oxidase-B (MAO-B) inhibitor.

Another aspect of the present invention is a method for the treatment, or delay, inhibition, or suppression of the progression of, a neurodegenerative disease, wherein the method comprises administering (e.g., orally) a c-Abl inhibitor (vodobatinib or a pharmaceutically acceptable salt thereof) to a subject (e.g., a human subject) in need thereof in an amount sufficient to achieve a ratio of mean CSF Cₘₐₓ to IC₅₀ of c-Abl inhibition of not less than 1, wherein the subject is not concomitantly administering symptomatic medication for Parkinson's disease.

Yet another aspect of the present invention is a method for the treatment, or delay, inhibition, or suppression of the progression of, a neurodegenerative disease, wherein the method comprises administering (e.g., orally) a c-Abl inhibitor to a subject (e.g., a human subject) in need thereof in an amount sufficient to achieve a ratio of mean CSF Cₘₐₓ to IC₅₀ of c-Abl inhibition of not less than 1, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Yet another aspect of the present invention is a method for the treatment, or delay, inhibition, or suppression of the progression of, a neurodegenerative disease, wherein the method comprises administering (e.g., orally) a compound of Formula I or a pharmaceutically acceptable salt thereof to a subject (e.g., a human subject) in need thereof in an amount sufficient to achieve:
i) a mean plasma AUC₀₋₂₄ ranging from about 5,000 ng*h/mL to about 75,000 ng*h/mL;
ii) a plasma Cₘₐₓ ranging from about 500 ng/mL to about 15,000 ng/mL; or
iii) a mean plasma AUC₀₋₂₄ ranging from about 5,000 ng*h/mL to about 75,000 ng*h/mL and a plasma Cₘₐₓ ranging from about 500 ng/mL to about 15,000 ng/mL.

Yet another aspect of the present invention is a method for the treatment, or delay, inhibition, or suppression of the progression of, a neurodegenerative disease, wherein the method comprises administering (e.g., orally) a compound of Formula I or a pharmaceutically acceptable salt thereof to a subject (e.g., a human subject) in need thereof in an amount sufficient to achieve:
i) a mean plasma AUC₀₋₂₄ ranging from about 5,000 ng*h/mL to about 75,000 ng*h/mL;
ii) a plasma Cₘₐₓ ranging from about 500 ng/mL to about 15,000 ng/mL; or
iii) a mean plasma AUC₀₋₂₄ ranging from about 5,000 ng*h/mL to about 75,000 ng*h/mL and a plasma Cₘₐₓ ranging from about 500 ng/mL to about 15,000 ng/mL,
wherein the subject exhibits a disease severity according to modified Hoehn & Yahr stage≤2.

Yet another aspect of the present invention is a method for the treatment, or delay, inhibition, or suppression of the progression of, a neurodegenerative disease, wherein the method comprises administering (e.g., orally) a compound of Formula I or a pharmaceutically acceptable salt thereof to a subject (e.g., a human subject) in need thereof in an amount sufficient to achieve:
i) a mean plasma AUC₀₋₂₄ ranging from about 5,000 ng*h/mL to about 75,000 ng*h/mL;
ii) a plasma Cₘₐₓ ranging from about 500 ng/mL to about 15,000 ng/mL; or
iii) a mean plasma AUC₀₋₂₄ ranging from about 5,000 ng*h/mL to about 75,000 ng*h/mL and a plasma Cₘₐₓ ranging from about 500 ng/mL to about 15,000 ng/mL,
wherein the subject exhibits a Montreal cognitive assessment score of at least 25.

Yet another aspect of the present invention is a method for the treatment, or delay, inhibition, or suppression of the progression of, a neurodegenerative disease, wherein the method comprises administering (e.g., orally) a compound of Formula I or a pharmaceutically acceptable salt thereof to a subject (e.g., a human subject) in need thereof in an amount sufficient to achieve:
i) a mean plasma AUC₀₋₂₄ ranging from about 5,000 ng*h/mL to about 75,000 ng*h/mL;
ii) a plasma Cₘₐₓ ranging from about 500 ng/mL to about 15,000 ng/mL; or
iii) a mean plasma AUC₀₋₂₄ ranging from about 5,000 ng*h/mL to about 75,000 ng*h/mL and a plasma Cₘₐₓ ranging from about 500 ng/mL to about 15,000 ng/mL,
wherein the subject exhibits a Dopamine Transporter Single Photon Emission Computed Tomography (DaT SPECT) consistent with the diagnosis of Parkinson's disease.

Yet another aspect of the present invention is a method for the treatment, or delay, inhibition, or suppression of the progression of, a neurodegenerative disease, wherein the method comprises administering (e.g., orally) a compound of Formula I or a pharmaceutically acceptable salt thereof to a subject (e.g., a human subject) in need thereof in an amount sufficient to achieve:
i) a mean plasma AUC₀₋₂₄ ranging from about 5,000 ng*h/mL to about 75,000 ng*h/mL;
ii) a plasma Cₘₐₓ ranging from about 500 ng/mL to about 15,000 ng/mL; or
iii) a mean plasma AUC₀₋₂₄ ranging from about 5,000 ng*h/mL to about 75,000 ng*h/mL and a plasma Cₘₐₓ ranging from about 500 ng/mL to about 15,000 ng/mL,
wherein the subject is receiving treatment with a monoamine oxidase-B (MAO-B) inhibitor.

Yet another aspect of the present invention is a method for the treatment, or delay, inhibition, or suppression of the progression of, a neurodegenerative disease, wherein the method comprises administering (e.g., orally) a compound of Formula I or a pharmaceutically acceptable salt thereof to a subject (e.g., a human subject) in need thereof in an amount sufficient to achieve:
i) a mean plasma AUC₀₋₂₄ ranging from about 5,000 ng*h/mL to about 75,000 ng*h/mL;
ii) a plasma Cₘₐₓ ranging from about 500 ng/mL to about 15,000 ng/mL; or
iii) a mean plasma AUC₀₋₂₄ ranging from about 5,000 ng*h/mL to about 75,000 ng*h/mL and a plasma Cₘₐₓ ranging from about 500 ng/mL to about 15,000 ng/mL,
wherein the subject is not concomitantly administering symptomatic medication for Parkinson's disease.

Another aspect of the present invention is a method for the treatment, or delay, inhibition, or suppression of the progression of, a neurodegenerative disease, wherein the method comprises administering (e.g., orally) a compound of Formula I or a pharmaceutically acceptable salt thereof to a subject (e.g., a human subject) in need thereof in an amount sufficient achieve:
i) a mean plasma AUC₀₋₂₄ ranging from about 5,000 ng*h/mL to about 75,000 ng*h/mL;
ii) a plasma Cₘₐₓ ranging from about 500 ng/mL to about 15,000 ng/mL; or
iii) a mean plasma AUC₀₋₂₄ ranging from about 5,000 ng*h/mL to about 75,000 ng*h/mL and a plasma Cₘₐₓ ranging from about 500 ng/mL to about 15,000 ng/mL;
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Yet another aspect of the present invention is a method for the treatment, or delay, inhibition, or suppression of the progression of, a neurodegenerative disease, wherein the method comprises administering (e.g., orally) a c-Abl inhibitor to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d).

Yet another aspect of the present invention is a method for the treatment, or delay, inhibition, or suppression of the progression of, a neurodegenerative disease, wherein the method comprises administering (e.g., orally) a c-Abl inhibitor to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject exhibits a disease severity according to modified Hoehn & Yahr stage≤2.

Yet another aspect of the present invention is a method for the treatment, or delay, inhibition, or suppression of the progression of, a neurodegenerative disease, wherein the method comprises administering (e.g., orally) a c-Abl inhibitor to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject exhibits a Montreal cognitive assessment score of at least 25.

Yet another aspect of the present invention is a method for the treatment, or delay, inhibition, or suppression of the progression of, a neurodegenerative disease, wherein the method comprises administering (e.g., orally) a c-Abl inhibitor to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject exhibits a Dopamine Transporter Single Photon Emission Computed Tomography (DaT SPECT) consistent with the diagnosis of Parkinson's disease.

Yet another aspect of the present invention is a method for the treatment, or delay, inhibition, or suppression of the progression of, a neurodegenerative disease, wherein the method comprises administering (e.g., orally) a c-Abl inhibitor to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject is receiving treatment with a monoamine oxidase-B (MAO-B) inhibitor.

Yet another aspect of the present invention is a method for the treatment, or delay, inhibition, or suppression of the progression of, a neurodegenerative disease, wherein the method comprises administering (e.g., orally) a c-Abl inhibitor to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject is not concomitantly administering symptomatic medication for Parkinson's disease.

Yet another aspect is a method for the treatment, or delay, inhibition, or suppression of the progression of, a neurodegenerative disease, wherein the method comprises administering (e.g., orally) a c-Abl inhibitor to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Yet another aspect of the present invention is a method for the treatment, or delay, inhibition, or suppression of the progression of, a neurodegenerative disease, wherein the method comprises orally administering a compound of Formula I or a pharmaceutically acceptable salt thereof to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d).

Yet another aspect of the present invention is a method for the treatment, or delay, inhibition, or suppression of the progression of, a neurodegenerative disease, wherein the method comprises orally administering a compound of Formula I or a pharmaceutically acceptable salt thereof to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject exhibits a disease severity according to modified Hoehn & Yahr stage≤2.

Yet another aspect of the present invention is a method for the treatment, or delay, inhibition, or suppression of the progression of, a neurodegenerative disease, wherein the method comprises orally administering a compound of Formula I or a pharmaceutically acceptable salt thereof to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject exhibits a Montreal cognitive assessment score of at least 25.

Yet another aspect of the present invention is a method for the treatment, or delay, inhibition, or suppression of the progression of, a neurodegenerative disease, wherein the method comprises administering (e.g. orally) a compound of Formula I or a pharmaceutically acceptable salt thereof to a subject (e.g., a human subject) in need thereof in an amount sufficient to cross the blood-brain barrier.

Yet another aspect of the present invention is a method for the treatment, or delay, inhibition, or suppression of the progression of, a neurodegenerative disease, wherein the method comprises administering (e.g. orally) a compound of Formula I or a pharmaceutically acceptable salt thereof to a subject (e.g., a human subject) in need thereof, wherein the subject has had an initial diagnosis of Parkinson's disease.

Yet another aspect of the present invention is a method for the treatment, or delay, inhibition, or suppression of the progression of, a neurodegenerative disease, wherein the method comprises administering (e.g. orally) a compound of Formula I or a pharmaceutically acceptable salt thereof to a subject (e.g., a human subject) in need thereof, wherein the subject has had an initial diagnosis of Parkinson's disease within 3 years of initiating treatment with a compound of Formula I or a pharmaceutically acceptable salt thereof.

Yet another aspect of the present invention is a method for the treatment, or delay, inhibition, or suppression of the progression of, a neurodegenerative disease, wherein the method comprises orally administering a compound of Formula I or a pharmaceutically acceptable salt thereof to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject exhibits a Dopamine Transporter Single Photon Emission Computed Tomography (DaT SPECT) consistent with the diagnosis of Parkinson's disease.

Yet another aspect of the present invention is a method for the treatment, or delay, inhibition, or suppression of the progression of, a neurodegenerative disease, wherein the method comprises orally administering a compound of Formula I or a pharmaceutically acceptable salt thereof to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject is receiving treatment with a monoamine oxidase-B (MAO-B) inhibitor.

Yet another aspect of the present invention is a method for the treatment, or delay, inhibition, or suppression of the progression of, a neurodegenerative disease, wherein the method comprises orally administering a compound of Formula I or a pharmaceutically acceptable salt thereof to a subject (e.g., a human subject) in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject is not concomitantly administering symptomatic medication for Parkinson's disease.

Another aspect is a method for the treatment, or delay, inhibition, or suppression of the progression of, a neurodegenerative disease, wherein the method comprises orally administering a compound of Formula I or a pharmaceutically acceptable salt thereof to a subject in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d);
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect is a method for the treatment, or delay, inhibition, or suppression of the progression of, a neurodegenerative disease, wherein the method comprises administering (e.g., orally) to a subject (e.g., a human subject) in need thereof a c-Abl inhibitor in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f).

Another aspect is a method for the treatment, or delay, inhibition, or suppression of the progression of, a neurodegenerative disease, wherein the method comprises administering (e.g., orally) to a subject (e.g., a human subject) in need thereof a c-Abl inhibitor in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a disease severity according to modified Hoehn & Yahr stage≤2.

Another aspect is a method for the treatment, or delay, inhibition, or suppression of the progression of, a neurodegenerative disease, wherein the method comprises administering (e.g., orally) to a subject (e.g., a human subject) in need thereof a c-Abl inhibitor in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a Montreal cognitive assessment score of at least 25.

Another aspect is a method for the treatment, or delay, inhibition, or suppression of the progression of, a neurodegenerative disease, wherein the method comprises administering (e.g., orally) to a subject (e.g., a human subject) in need thereof a c-Abl inhibitor in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a Dopamine Transporter Single Photon Emission Computed Tomography (DaT SPECT) consistent with the diagnosis of Parkinson's disease.

Another aspect is a method for the treatment, or delay, inhibition, or suppression of the progression of, a neurodegenerative disease, wherein the method comprises administering (e.g., orally) to a subject (e.g., a human subject) in need thereof a c-Abl inhibitor in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject is receiving treatment with a monoamine oxidase-B (MAO-B) inhibitor.

Another aspect is a method for the treatment, or delay, inhibition, or suppression of the progression of, a neurodegenerative disease, wherein the method comprises administering (e.g., orally) to a subject (e.g., a human subject) in need thereof a c-Abl inhibitor in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject is not concomitantly administering symptomatic medication for Parkinson's disease.

Yet another aspect is a method for the treatment, or delay, inhibition, or suppression of the progression of, a neurodegenerative disease, wherein the method comprises administering (e.g., orally) to a subject (e.g., a human subject) in need thereof a c-Abl inhibitor in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f);
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect of the present invention is a method for the treatment, or delay, inhibition, or suppression of the progression of, a neurodegenerative disease, wherein the method comprises administering (e.g., orally) to a subject (e.g., a human subject) in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f).

Yet another aspect of the present invention is a method for the treatment, or delay, inhibition, or suppression of the progression of, a neurodegenerative disease, wherein the method comprises administering (e.g., orally) to a subject (e.g., a human subject) in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a disease severity according to modified Hoehn & Yahr stage≤2.

Yet another aspect of the present invention is a method for the treatment, or delay, inhibition, or suppression of the progression of, a neurodegenerative disease, wherein the method comprises administering (e.g., orally) to a subject (e.g., a human subject) in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a Montreal cognitive assessment score of at least 25.

Yet another aspect of the present invention is a method for the treatment, or delay, inhibition, or suppression of the progression of, a neurodegenerative disease, wherein the method comprises administering (e.g., orally) to a subject (e.g., a human subject) in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a Dopamine Transporter Single Photon Emission Computed Tomography (DaT SPECT) consistent with the diagnosis of Parkinson's disease.

Yet another aspect of the present invention is a method for the treatment, or delay, inhibition, or suppression of the progression of, a neurodegenerative disease, wherein the method comprises administering (e.g., orally) to a subject (e.g., a human subject) in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject is receiving treatment with a monoamine oxidase-B (MAO-B) inhibitor.

Yet another aspect of the present invention is a method for the treatment, or delay, inhibition, or suppression of the progression of, a neurodegenerative disease, wherein the method comprises administering (e.g., orally) to a subject (e.g., a human subject) in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject is not concomitantly administering symptomatic medication for Parkinson's disease.

Yet another aspect of the invention is a method for the treatment, or delay, inhibition, or suppression of the progression of, a neurodegenerative disease, wherein the method comprises administering (e.g., orally) to a subject in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f);
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Yet another aspect is the use of a c-Abl inhibitor for the preparation of a medicament for the treatment, or delay, inhibition, or suppression of the progression of, a neurodegenerative disease in a subject (e.g., a human subject) in need thereof, wherein the medicament comprises the c-Abl inhibitor in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f).

Yet another aspect is the use of a c-Abl inhibitor for the preparation of a medicament for the treatment, or delay, inhibition, or suppression of the progression of, a neurodegenerative disease in a subject (e.g., a human subject) in need thereof, wherein the medicament comprises the c-Abl inhibitor in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a disease severity according to modified Hoehn & Yahr stage≤2.

Yet another aspect is the use of a c-Abl inhibitor for the preparation of a medicament for the treatment, or delay, inhibition, or suppression of the progression of, a neurodegenerative disease in a subject (e.g., a human subject) in need thereof, wherein the medicament comprises the c-Abl inhibitor in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a Montreal cognitive assessment score of at least 25.

Yet another aspect is the use of a c-Abl inhibitor for the preparation of a medicament for the treatment, or delay, inhibition, or suppression of the progression of, a neurodegenerative disease in a subject (e.g., a human subject) in need thereof, wherein the medicament comprises the c-Abl inhibitor in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a Dopamine Transporter Single Photon Emission Computed Tomography (DaT SPECT) consistent with the diagnosis of Parkinson's disease.

Yet another aspect is the use of a c-Abl inhibitor for the preparation of a medicament for the treatment, or delay, inhibition, or suppression of the progression of, a neurodegenerative disease in a subject (e.g., a human subject) in need thereof, wherein the medicament comprises the c-Abl inhibitor in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject is receiving treatment with a monoamine oxidase-B (MAO-B) inhibitor.

Yet another aspect is the use of a c-Abl inhibitor for the preparation of a medicament for the treatment, or delay, inhibition, or suppression of the progression of, a neurodegenerative disease in a subject (e.g., a human subject) in need thereof, wherein the medicament comprises the c-Abl inhibitor in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject is not concomitantly administering symptomatic medication for Parkinson's disease.

Yet another aspect is the use of a c-Abl inhibitor for the preparation of a medicament for the treatment, or delay, inhibition, or suppression of the progression of, a neurodegenerative disease in a subject (e.g., a human subject) in need thereof, wherein the medicament comprises the c-Abl inhibitor in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f);
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Yet another aspect of the present invention is the use of a compound of Formula I or a pharmaceutically acceptable salt thereof for the preparation of a medicament for the treatment, or delay, inhibition, or suppression of the progression of, a neurodegenerative disease in a subject (e.g., a human subject) in need thereof, wherein the medicament comprises the compound of Formula I or a pharmaceutically acceptable salt thereof in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f).

Yet another aspect of the present invention is a compound of Formula I or a pharmaceutically acceptable salt thereof for use in the preparation of a medicament for the treatment, or delay, inhibition, or suppression of the progression of, a neurodegenerative disease in a subject (e.g., a human subject) in need thereof, wherein the medicament comprises the compound of Formula I or a pharmaceutically acceptable salt thereof in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a disease severity according to modified Hoehn & Yahr stage≤2.

Yet another aspect of the present invention is a compound of Formula I or a pharmaceutically acceptable salt thereof for use in the preparation of a medicament for the treatment, or delay, inhibition, or suppression of the progression of, a neurodegenerative disease in a subject (e.g., a human subject) in need thereof, wherein the medicament comprises the compound of Formula I or a pharmaceutically acceptable salt thereof in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a Montreal cognitive assessment score of at least 25.

Yet another aspect of the present invention is a compound of Formula I or a pharmaceutically acceptable salt thereof for use in the preparation of a medicament for the treatment, or delay, inhibition, or suppression of the progression of, a neurodegenerative disease in a subject (e.g., a human subject) in need thereof, wherein the medicament comprises the compound of Formula I or a pharmaceutically acceptable salt thereof in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject exhibits a Dopamine Transporter Single Photon Emission Computed Tomography (DaT SPECT) consistent with the diagnosis of Parkinson's disease.

Yet another aspect of the present invention is a compound of Formula I or a pharmaceutically acceptable salt thereof for use in the preparation of a medicament for the treatment, or delay, inhibition, or suppression of the progression of, a neurodegenerative disease in a subject (e.g., a human subject) in need thereof, wherein the medicament comprises the compound of Formula I or a pharmaceutically acceptable salt thereof in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject is receiving treatment with a monoamine oxidase-B (MAO-B) inhibitor.

Yet another aspect of the present invention is a compound of Formula I or a pharmaceutically acceptable salt thereof for use in the preparation of a medicament for the treatment, or delay, inhibition, or suppression of the progression of, a neurodegenerative disease in a subject (e.g., a human subject) in need thereof, wherein the medicament comprises the compound of Formula I or a pharmaceutically acceptable salt thereof in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f),
wherein the subject is not concomitantly administering symptomatic medication for Parkinson's disease.

Yet another aspect of the present invention is a compound of Formula I or a pharmaceutically acceptable salt thereof for use in the preparation of a medicament for the treatment, or delay, inhibition, or suppression of the progression of, a neurodegenerative disease in a subject (e.g., a human subject) in need thereof, wherein the medicament comprises the compound of Formula I or a pharmaceutically acceptable salt thereof in a sufficient amount to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL;
f) achieve a ratio of mean CSF Cₘₐₓ to IC₅₀ of c-Abl inhibition of not less than 1; or
g) any combination of a), b), c), d), e) and f);
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect of the present invention is a compound of Formula I or a pharmaceutically acceptable salt thereof for use in the treatment, or delay, inhibition, or suppression of the progression of, a neurodegenerative disease in a subject (e.g., a human subject), comprising administering a compound of Formula I or a pharmaceutically acceptable salt thereof in an amount sufficient to achieve a CSF Cₘₐₓ to IC₅₀ of c-Abl inhibition ratio not less than 1.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a CSF Cₘₐₓ to IC₅₀ of c-Abl inhibition ratio not less than 1.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a CSF Cₘₐₓ to IC₅₀ of c-Abl inhibition ratio not less than 1, wherein the subject exhibits a disease severity according to modified Hoehn & Yahr stage≤2.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a CSF Cₘₐₓ to IC₅₀ of c-Abl inhibition ratio not less than 1, wherein the subject exhibits a Montreal cognitive assessment score of at least 25.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a CSF Cₘₐₓ to IC₅₀ of c-Abl inhibition ratio not less than 1, wherein the subject exhibits a Dopamine Transporter Single Photon Emission Computed Tomography (DaT SPECT) consistent with the diagnosis of Parkinson's disease.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a CSF Cₘₐₓ to IC₅₀ of c-Abl inhibition ratio not less than 1, wherein the subject is receiving treatment with a monoamine oxidase-B (MAO-B) inhibitor.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a CSF Cₘₐₓ to IC₅₀ of c-Abl inhibition ratio not less than 1, wherein the subject is not concomitantly administering symptomatic medication for Parkinson's disease.

Yet another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof safely achieves a CSF Cₘₐₓ to IC₅₀ of c-Abl inhibition ratio not less than 1;
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect of the present invention is a method for the reduction of the rate of progression of an early-stage neurodegenerative disease in a subject (e.g., a human subject), wherein the method comprises administering (e.g., orally) to the subject a compound of Formula I or a pharmaceutically acceptable salt thereof to the subject in need thereof in an amount sufficient to achieve a CSF Cₘₐₓ to IC₅₀ of c-Abl inhibition ratio of not less than 1.

Another aspect is a method for the reduction of the rate of progression of Parkinson's disease, preferably early-stage Parkinson's disease, in a subject (e.g., a human subject), wherein the method comprises administering (e.g., orally) to the subject a compound of Formula I or a pharmaceutically acceptable salt thereof in an amount sufficient to achieve a CSF Cₘₐₓ to IC₅₀ of c-Abl inhibition ratio of not less than 1.

Another aspect is a method for the reduction of the rate of progression of Parkinson's disease, preferably early-stage Parkinson's disease, in a subject (e.g., a human subject), wherein the method comprises administering (e.g., orally) to the subject a compound of Formula I or a pharmaceutically acceptable salt thereof in an amount sufficient to achieve a CSF Cₘₐₓ to IC₅₀ of c-Abl inhibition ratio of not less than 1, wherein the subject exhibits a disease severity according to modified Hoehn & Yahr stage≤2.

Another aspect is a method for the reduction of the rate of progression of Parkinson's disease, preferably early-stage Parkinson's disease, in a subject (e.g., a human subject), wherein the method comprises administering (e.g., orally) to the subject a compound of Formula I or a pharmaceutically acceptable salt thereof in an amount sufficient to achieve a CSF Cₘₐₓ to IC₅₀ of c-Abl inhibition ratio of not less than 1, wherein the subject exhibits a Montreal cognitive assessment score of at least 25.

Another aspect is a method for the reduction of the rate of progression of Parkinson's disease, preferably early-stage Parkinson's disease, in a subject (e.g., a human subject), wherein the method comprises administering (e.g., orally) to the subject a compound of Formula I or a pharmaceutically acceptable salt thereof in an amount sufficient to achieve a CSF Cₘₐₓ to IC₅₀ of c-Abl inhibition ratio of not less than 1, wherein the subject exhibits a Dopamine Transporter Single Photon Emission Computed Tomography (DaT SPECT) consistent with the diagnosis of Parkinson's disease.

Another aspect is a method for the reduction of the rate of progression of Parkinson's disease, preferably early-stage Parkinson's disease, in a subject (e.g., a human subject), wherein the method comprises administering (e.g., orally) to the subject a compound of Formula I or a pharmaceutically acceptable salt thereof in an amount sufficient to achieve a CSF Cₘₐₓ to IC₅₀ of c-Abl inhibition ratio of not less than 1, wherein the subject is receiving treatment with a monoamine oxidase-B (MAO-B) inhibitor.

Another aspect is a method for the reduction of the rate of progression of Parkinson's disease, preferably early-stage Parkinson's disease, in a subject (e.g., a human subject), wherein the method comprises administering (e.g., orally) to the subject a compound of Formula I or a pharmaceutically acceptable salt thereof in an amount sufficient to achieve a CSF Cₘₐₓ to IC₅₀ of c-Abl inhibition ratio of not less than 1, wherein the subject is not concomitantly administering symptomatic medication for Parkinson's disease.

Yet another aspect of the present invention is a method for the reduction of the rate of progression of an early-stage neurodegenerative disease, preferably early-stage Parkinson's disease, in a subject (e.g., a human subject), wherein the method comprises administering (e.g., orally) to the subject a compound of Formula I or a pharmaceutically acceptable salt thereof in an amount sufficient to achieve a CSF Cₘₐₓ to IC₅₀ of c-Abl inhibition ratio of not less than 1;
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect of the present invention is a method for the reduction of the rate of progression of an early-stage neurodegenerative disease, preferably early-stage Parkinson's disease, wherein the method comprises administering (e.g., orally) to a subject in need thereof (e.g., a human subject) a compound of Formula I or a pharmaceutically acceptable salt thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL.

Another aspect of the present invention is a method for the reduction of the rate of progression of an early-stage neurodegenerative disease, preferably early-stage Parkinson's disease, wherein the method comprises administering (e.g., orally) to a subject in need thereof (e.g., a human subject) a compound of Formula I or a pharmaceutically acceptable salt thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject exhibits a disease severity according to modified Hoehn & Yahr stage≤2.

Another aspect of the present invention is a method for the reduction of the rate of progression of an early-stage neurodegenerative disease, preferably early-stage Parkinson's disease, wherein the method comprises administering (e.g., orally) to a subject in need thereof (e.g., a human subject) a compound of Formula I or a pharmaceutically acceptable salt thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject exhibits a Montreal cognitive assessment score of at least 25.

Another aspect of the present invention is a method for the reduction of the rate of progression of an early-stage neurodegenerative disease, preferably early-stage Parkinson's disease, wherein the method comprises administering (e.g., orally) to a subject in need thereof (e.g., a human subject) a compound of Formula I or a pharmaceutically acceptable salt thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject exhibits a Dopamine Transporter Single Photon Emission Computed Tomography (DaT SPECT) consistent with the diagnosis of Parkinson's disease.

Another aspect of the present invention is a method for the reduction of the rate of progression of an early-stage neurodegenerative disease, preferably early-stage Parkinson's disease, wherein the method comprises administering (e.g., orally) to a subject in need thereof (e.g., a human subject) a compound of Formula I or a pharmaceutically acceptable salt thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject is receiving treatment with a monoamine oxidase-B (MAO-B) inhibitor.

Another aspect of the present invention is a method for the reduction of the rate of progression of an early-stage neurodegenerative disease, preferably early-stage Parkinson's disease, wherein the method comprises administering (e.g., orally) to a subject in need thereof (e.g., a human subject) a compound of Formula I or a pharmaceutically acceptable salt thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject is not concomitantly administering symptomatic medication for Parkinson's disease.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a mean plasma AUC₀₋₂₄ ranging from about 30,000 ng*h/mL to about 50,000 ng*h/mL and/or a plasma Cₘₐₓ ranging from about 3,000 ng/mL to about 7,000 ng/mL.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a mean plasma AUC₀₋₂₄ ranging from about 40,000 ng*h/mL to about 60,000 ng*h/mL and/or a plasma Cₘₐₓ ranging from about 5,000 ng/mL to about 9,000 ng/mL.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a mean plasma AUC₀₋₂₄ ranging from about 15,000 ng*h/mL to about 40,000 ng*h/mL and/or a plasma Cₘₐₓ ranging from about 1,000 ng/mL to about 4,000 ng/mL.

Another aspect of the present invention is a compound of Formula I or a pharmaceutically acceptable salt thereof for use in the treatment, or delay, inhibition, or suppression of the progression of, a neurodegenerative disease, preferably Parkinson's disease, more preferably early-stage Parkinson's disease in a subject (e.g., a human subject), wherein the compound of Formula I or a pharmaceutically acceptable salt thereof achieves a mean plasma AUC₀₋₂₄ ranging from about 25,000 ng*h/mL to about 50,000 ng*h/mL and/or a plasma Cₘₐₓ ranging from about 2,000 ng/mL to about 6,000 ng/mL.

Yet another aspect of the present invention is a method for the reduction of the rate of progression of an early-stage neurodegenerative disease, preferably early-stage Parkinson's disease, wherein the method comprises administering (e.g., orally) to a subject in need thereof (e.g., a human subject) a compound of Formula I or a pharmaceutically acceptable salt thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL;
wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect of the present invention is a method for the reduction of the rate of progression of an early-stage neurodegenerative disease, wherein the method comprises administering (e.g., orally) to a subject in need thereof (e.g., a human subject) a compound of Formula I or a pharmaceutically acceptable salt thereof in an amount sufficient to:
a) achieve a CSF Cₘₐₓ ranging from about 0.4 ng/mL to about 7.0 ng/mL;
b) achieve a CSF concentration at least about 0.2 ng/mL to about 2.7 ng/mL;
c) achieve a ratio of mean CSF Cₘₐₓ to IC₅₀ of c-Abl inhibition of not less than 1; or
e) any combination of a), b), and c).

Another aspect of the present invention is a method for the reduction of the rate of progression of an early-stage neurodegenerative disease, wherein the method comprises administering (e.g., orally) to a subject in need thereof (e.g., a human subject) a compound of Formula I or a pharmaceutically acceptable salt thereof in an amount sufficient to:
a) achieve a CSF Cₘₐₓ ranging from about 0.4 ng/mL to about 7.0 ng/mL;
b) achieve a CSF concentration at least about 0.2 ng/mL to about 2.7 ng/mL;
c) achieve a ratio of mean CSF Cₘₐₓ to IC₅₀ of c-Abl inhibition of not less than 1; or
d) any combination of a), b), and c),
wherein the subject exhibits a disease severity according to modified Hoehn & Yahr stag≤2.

Another aspect of the present invention is a method for the reduction of the rate of progression of an early-stage neurodegenerative disease, wherein the method comprises administering (e.g., orally) to a subject in need thereof (e.g., a human subject) a compound of Formula I or a pharmaceutically acceptable salt thereof in an amount sufficient to:
a) achieve a CSF Cₘₐₓ ranging from about 0.4 ng/mL to about 7.0 ng/mL;
b) achieve a CSF concentration at least about 0.2 ng/mL to about 2.7 ng/mL;
c) achieve a ratio of mean CSF Cₘₐₓ to IC₅₀ of c-Abl inhibition of not less than 1; or
d) any combination of a), b), and c),
wherein the subject exhibits a Montreal cognitive assessment score of at least 25.

Another aspect of the present invention is a method for the reduction of the rate of progression of an early-stage neurodegenerative disease, wherein the method comprises administering (e.g., orally) to a subject in need thereof (e.g., a human subject) a compound of Formula I or a pharmaceutically acceptable salt thereof in an amount sufficient to:
a) achieve a CSF Cₘₐₓ ranging from about 0.4 ng/mL to about 7.0 ng/mL;
b) achieve a CSF concentration at least about 0.2 ng/mL to about 2.7 ng/mL;
c) achieve a ratio of mean CSF Cₘₐₓ to IC₅₀ of c-Abl inhibition of not less than 1; or
d) any combination of a), b), and c),
wherein the subject exhibits a Dopamine Transporter Single Photon Emission Computed Tomography (DaT SPECT) consistent with the diagnosis of Parkinson's disease.

Another aspect of the present invention is a method for the reduction of the rate of progression of an early-stage neurodegenerative disease, wherein the method comprises administering (e.g., orally) to a subject in need thereof (e.g., a human subject) a compound of Formula I or a pharmaceutically acceptable salt thereof in an amount sufficient to:
a) achieve a CSF Cₘₐₓ ranging from about 0.4 ng/mL to about 7.0 ng/mL;
b) achieve a CSF concentration at least about 0.2 ng/mL to about 2.7 ng/mL;
c) achieve a ratio of mean CSF Cₘₐₓ to IC₅₀ of c-Abl inhibition of not less than 1; or
d) any combination of a), b), and c),
wherein the subject is receiving treatment with a monoamine oxidase-B (MAO-B) inhibitor.

Another aspect of the present invention is a method for the reduction of the rate of progression of an early-stage neurodegenerative disease, wherein the method comprises administering (e.g., orally) to a subject in need thereof (e.g., a human subject) a compound of Formula I or a pharmaceutically acceptable salt thereof in an amount sufficient to:
a) achieve a CSF Cₘₐₓ ranging from about 0.4 ng/mL to about 7.0 ng/mL;
b) achieve a CSF concentration at least about 0.2 ng/mL to about 2.7 ng/mL;
c) achieve a ratio of mean CSF Cₘₐₓ to IC₅₀ of c-Abl inhibition of not less than 1; or
d) any combination of a), b), and c),
wherein the subject is not concomitantly administering symptomatic medication for Parkinson's disease.

Yet another aspect is a method for reduction of the rate of progression of early-stage neurodegenerative disease, wherein the method comprises administering (e.g., orally) a compound of Formula I or a pharmaceutically acceptable salt thereof to a subject in need thereof in an amount sufficient to:
a) achieve a CSF Cₘₐₓ ranging from about 0.4 ng/mL to about 7.0 ng/mL;
b) achieve a CSF concentration of at least about 0.2 ng/mL to about 2.7 ng/mL; and/or
c) provide for a ratio of mean CSF Cₘₐₓ to IC₅₀ of c-Abl inhibition of not less than 1;
wherein the subject exhibits one or more (such as 2, 3, 4, 5, 6, 7, 8, 9 or 10) of
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Another aspect is a pharmaceutical composition comprising a compound of Formula I or a pharmaceutically acceptable salt thereof, wherein the composition comprises the compound of Formula I or a pharmaceutically acceptable salt thereof in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1,
and wherein the composition is used in the treatment, or delay, inhibition, or suppression of the progression of, a neurodegenerative disease.

Yet another aspect is a pharmaceutical composition comprising a compound of Formula I or a pharmaceutically acceptable salt thereof for the treatment, or delay, inhibition, or suppression of the progression of, a neurodegenerative disease in a subject, wherein the composition comprises the compound of Formula I or a pharmaceutically acceptable salt thereof in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1,
wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

According to present invention the Neurodegenerative diseases (ND) referred herein includes, but not limited to, Parkinson's disease (PD), Alzheimer's disease (AD) or synucleinopathies, such as Lewy body dementia (LBD), Rapid eye movement (REM) sleep behaviour disorder and Multiple system atrophy (MSA). In a preferred embodiment, the neurodegenerative disease is Parkinson Disease, more preferably early-stage Parkinson Disease. In another embodiment, the neurodegenerative disease includes early-stage neurodegenerative disease including, but not limited to, early-stage Parkinson's disease (PD), early stage Alzheimer's disease (AD) and early-stage of synucleinopathies including early-stage Lewy body dementia (LBD), early-stage Rapid eye movement (REM) sleep behaviour disorder and early-stage Multiple system atrophy (MSA).

Another aspect is a method for the treatment, or delay, inhibition, or suppression of the progression of, Parkinson's disease (PD) in a subject in need thereof, comprising administering (e.g., orally) a compound of Formula I or a pharmaceutically acceptable salt thereof in an amount sufficient to achieve a mean plasma AUC₀₋₂₄ ranging from about 5,000 ng*h/mL to about 75,000 ng*h/mL.

Yet another aspect is a method for the treatment, or delay, inhibition, or suppression of the progression of, Parkinson's disease (PD) in a subject in need thereof, comprising administering (e.g., orally) a compound of Formula I or a pharmaceutically acceptable salt thereof in an amount sufficient to achieve a mean plasma AUC₀₋₂₄ ranging from about 5,000 ng*h/mL to about 75,000 ng*h/mL, wherein the subject exhibits a disease severity according to modified Hoehn & Yahr stage≤2.

Yet another aspect is a method for the treatment, or delay, inhibition, or suppression of the progression of, Parkinson's disease (PD) in a subject in need thereof, comprising administering (e.g., orally) a compound of Formula I or a pharmaceutically acceptable salt thereof in an amount sufficient to achieve a mean plasma AUC₀₋₂₄ ranging from about 5,000 ng*h/mL to about 75,000 ng*h/mL, wherein the subject exhibits a Montreal cognitive assessment score of at least 25.

Yet another aspect is a method for the treatment, or delay, inhibition, or suppression of the progression of, Parkinson's disease (PD) in a subject in need thereof, comprising administering (e.g., orally) a compound of Formula I or a pharmaceutically acceptable salt thereof in an amount sufficient to achieve a mean plasma AUC₀₋₂₄ ranging from about 5,000 ng*h/mL to about 75,000 ng*h/mL, wherein the subject exhibits a Dopamine Transporter Single Photon Emission Computed Tomography (DaT SPECT) consistent with the diagnosis of Parkinson's disease.

Yet another aspect is a method for the treatment, or delay, inhibition, or suppression of the progression of, Parkinson's disease (PD) in a subject in need thereof, comprising administering (e.g., orally) a compound of Formula I or a pharmaceutically acceptable salt thereof in an amount sufficient to achieve a mean plasma AUC₀₋₂₄ ranging from about 5,000 ng*h/mL to about 75,000 ng*h/mL, wherein the subject is receiving treatment with a monoamine oxidase-B (MAO-B) inhibitor.

Yet another aspect is a method for the treatment, or delay, inhibition, or suppression of the progression of, Parkinson's disease (PD) in a subject in need thereof, comprising administering (e.g., orally) a compound of Formula I or a pharmaceutically acceptable salt thereof in an amount sufficient to achieve a mean plasma AUC₀₋₂₄ ranging from about 5,000 ng*h/mL to about 75,000 ng*h/mL, wherein the subject is not concomitantly administering symptomatic medication for Parkinson's disease.

Yet another aspect is a method for the treatment, or delay, inhibition, or suppression of the progression of, Parkinson's disease (PD) in a subject in need thereof, comprising administering (e.g., orally) a compound of Formula I or a pharmaceutically acceptable salt thereof in an amount sufficient to achieve a mean plasma AUC₀₋₂₄ ranging from about 5,000 ng*h/mL to about 75,000 ng*h/mL; wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a plasma Cₘₐₓ ranging from about 500 ng/mL to about 15,000 ng/mL.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a plasma Cₘₐₓ ranging from about 500 ng/mL to about 15,000 ng/mL, wherein the subject exhibits a disease severity according to modified Hoehn & Yahr stage≤2.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a plasma Cₘₐₓ ranging from about 500 ng/mL to about 15,000 ng/mL, wherein the subject exhibits a Montreal cognitive assessment score of at least 25.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a plasma Cₘₐₓ ranging from about 500 ng/mL to about 15,000 ng/mL, wherein the subject exhibits a Dopamine Transporter Single Photon Emission Computed Tomography (DaT SPECT) consistent with the diagnosis of Parkinson's disease.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a plasma Cₘₐₓ ranging from about 500 ng/mL to about 15,000 ng/mL, wherein the subject is receiving treatment with a monoamine oxidase-B (MAO-B) inhibitor.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a plasma Cₘₐₓ ranging from about 500 ng/mL to about 15,000 ng/mL, wherein the subject is not concomitantly administering symptomatic medication for Parkinson's disease.

Yet another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a plasma Cₘₐₓ ranging from about 500 ng/mL to about 15,000 ng/mL; wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a CSF Cₘₐₓ ranging from about 0.4 ng/mL to about 7.0 ng/mL.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a CSF Cₘₐₓ ranging from about 2 ng/mL to about 7 ng/mL.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a CSF Cₘₐₓ ranging from about 0.4 ng/mL to about 7.0 ng/mL, wherein the subject exhibits a disease severity according to modified Hoehn & Yahr stage≤2.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a CSF Cₘₐₓ ranging from about 0.4 ng/mL to about 7.0 ng/mL, wherein the subject exhibits a Montreal cognitive assessment score of at least 25.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a CSF Cₘₐₓ ranging from about 0.4 ng/mL to about 7.0 ng/mL, wherein the subject exhibits a Dopamine Transporter Single Photon Emission Computed Tomography (DaT SPECT) consistent with the diagnosis of Parkinson's disease.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a CSF Cₘₐₓ ranging from about 0.4 ng/mL to about 7.0 ng/mL, wherein the subject is receiving treatment with a monoamine oxidase-B (MAO-B) inhibitor.

Another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a CSF Cₘₐₓ ranging from about 0.4 ng/mL to about 7.0 ng/mL, wherein the subject is not concomitantly administering symptomatic medication for Parkinson's disease.

Yet another aspect is a method for the treatment, or delay, inhibition, or suppression of the progression of, Parkinson's disease in a subject in need thereof comprising administering (e.g., orally) a compound of Formula I or a pharmaceutically acceptable salt thereof in an amount sufficient to achieve a CSF Cₘₐₓ ranging from about 0.4 ng/mL to about 7.0 ng/mL; wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Another aspect is a method for the treatment, or delay, inhibition, or suppression of the progression of, Parkinson's disease, wherein the method comprises administering (e.g., orally) to a subject in need thereof a c-Abl inhibitor in an amount sufficient to achieve a ratio of mean CSF Cₘₐₓ to IC₅₀ of c-Abl inhibition of not less than 1.

Yet another aspect is a method for the treatment, or delay, inhibition, or suppression of the progression of, Parkinson's disease, wherein the method comprises administering (e.g., orally) to a subject in need thereof a c-Abl inhibitor in an amount sufficient to achieve a ratio of mean CSF Cₘₐₓ to IC₅₀ of c-Abl inhibition of not less than 1, wherein the subject exhibits a disease severity according to modified Hoehn & Yahr stage≤2.

Yet another aspect is a method for the treatment, or delay, inhibition, or suppression of the progression of, Parkinson's disease, wherein the method comprises administering (e.g., orally) to a subject in need thereof a c-Abl inhibitor in an amount sufficient to achieve a ratio of mean CSF Cₘₐₓ to IC₅₀ of c-Abl inhibition of not less than 1, wherein the subject exhibits a Montreal cognitive assessment score of at least 25.

Yet another aspect is a method for the treatment, or delay, inhibition, or suppression of the progression of, Parkinson's disease, wherein the method comprises administering (e.g., orally) to a subject in need thereof a c-Abl inhibitor in an amount sufficient to achieve a ratio of mean CSF Cₘₐₓ to IC₅₀ of c-Abl inhibition of not less than 1, wherein the subject exhibits a Dopamine Transporter Single Photon Emission Computed Tomography (DaT SPECT) consistent with the diagnosis of Parkinson's disease.

Yet another aspect is a method for the treatment, or delay, inhibition, or suppression of the progression of, Parkinson's disease, wherein the method comprises administering (e.g., orally) to a subject in need thereof a c-Abl inhibitor in an amount sufficient to achieve a ratio of mean CSF Cₘₐₓ to IC₅₀ of c-Abl inhibition of not less than 1, wherein the subject is receiving treatment with a monoamine oxidase-B (MAO-B) inhibitor.

Yet another aspect is a method for the treatment, or delay, inhibition, or suppression of the progression of, Parkinson's disease, wherein the method comprises administering (e.g., orally) to a subject in need thereof a c-Abl inhibitor in an amount sufficient to achieve a ratio of mean CSF Cₘₐₓ to IC₅₀ of c-Abl inhibition of not less than 1, wherein the subject is not concomitantly administering symptomatic medication for Parkinson's disease.

Yet another aspect is a method for the treatment, or delay, inhibition, or suppression of the progression of, Parkinson's disease, wherein the method comprises administering (e.g., orally) to a subject in need thereof a c-Abl inhibitor in an amount sufficient to achieve a ratio of mean CSF Cₘₐₓ to IC₅₀ of c-Abl inhibition of not less than 1; wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

One aspect is a method for the treatment, or delay, inhibition, or suppression of the progression of, Parkinson's disease, wherein the method comprises administering (e.g., orally) to a subject in need thereof a c-Abl inhibitor in an amount sufficient to achieve a ratio of mean CSF Cₘₐₓ to IC₅₀ of c-Abl inhibition of from 1 to 14.

In a preferred embodiment, a compound of Formula I or a pharmaceutically acceptable salt thereof is administered in an amount sufficient to achieve a ratio of Cₘₐₓ to IC₅₀ greater than 5.

One aspect is a method for the treatment, or delay, inhibition, or suppression of the progression of, neurodegenerative disease, preferably neurodegenerative disease is Parkinson's disease, or early-stage Parkinson's disease, wherein the method comprises administering to a subject (e.g., a human) in need thereof, a compound of Formula I or a pharmaceutically acceptable salt thereof, in an amount sufficient to achieve a ratio of mean CSF Cₘₐₓ to IC₅₀ of c-Abl inhibition of from 1 to 14.

One aspect is a method for the treatment, or delay, inhibition, or suppression of the progression of, neurodegenerative disease, preferably neurodegenerative disease is Parkinson's disease, or early-stage Parkinson's disease, wherein the method comprises administering to a subject in need thereof a c-Abl inhibitor, preferably the c-Abl inhibitor is a compound of Formula I or a pharmaceutically acceptable salt thereof, in an amount sufficient to achieve a ratio of mean CSF Cₘₐₓ to IC₅₀ of c-Abl inhibition of from 1 to 14.

One aspect is a method for the treatment, or delay, inhibition, or suppression of the progression of, a neurodegenerative disease, preferably neurodegenerative disease is Parkinson's disease, or early-stage Parkinson's disease, wherein the method comprises administering (e.g., orally) to a subject in need thereof a c-Abl inhibitor, preferably the c-Abl inhibitor is a compound of Formula I or a pharmaceutically acceptable salt thereof, in an amount sufficient to achieve a ratio of mean CSF Cₘₐₓ to IC₅₀ of c-Abl inhibition of from 1 to 14, wherein the subject exhibits a disease severity according to modified Hoehn & Yahr stage≤2.

One aspect is a method for the treatment, or delay, inhibition, or suppression of the progression of, a neurodegenerative disease, preferably neurodegenerative disease is Parkinson's disease, or early-stage Parkinson's disease, wherein the method comprises administering (e.g., orally) to a subject in need thereof a c-Abl inhibitor, preferably the c-Abl inhibitor is a compound of Formula I or a pharmaceutically acceptable salt thereof, in an amount sufficient to achieve a ratio of mean CSF Cₘₐₓ to IC₅₀ of c-Abl inhibition of from 1 to 14, wherein the subject exhibits a Montreal cognitive assessment score of at least 25.

One aspect is a method for the treatment, or delay, inhibition, or suppression of the progression of, a neurodegenerative disease, preferably neurodegenerative disease is Parkinson's disease, or early-stage Parkinson's disease, wherein the method comprises administering (e.g., orally) to a subject in need thereof a c-Abl inhibitor, preferably the c-Abl inhibitor is a compound of Formula I or a pharmaceutically acceptable salt thereof, in an amount sufficient to achieve a ratio of mean CSF Cₘₐₓ to IC₅₀ of c-Abl inhibition of from 1 to 14, wherein the subject exhibits a Dopamine Transporter Single Photon Emission Computed Tomography (DaT SPECT) consistent with the diagnosis of Parkinson's disease.

One aspect is a method for the treatment, or delay, inhibition, or suppression of the progression of, a neurodegenerative disease, preferably neurodegenerative disease is Parkinson's disease, or early-stage Parkinson's disease, wherein the method comprises administering (e.g., orally) to a subject in need thereof a c-Abl inhibitor, preferably the c-Abl inhibitor is a compound of Formula I or a pharmaceutically acceptable salt thereof, in an amount sufficient to achieve a ratio of mean CSF Cₘₐₓ to IC₅₀ of c-Abl inhibition of from 1 to 14, wherein the subject is receiving treatment with a monoamine oxidase-B (MAO-B) inhibitor.

One aspect is a method for the treatment, or delay, inhibition, or suppression of the progression of, a neurodegenerative disease, preferably neurodegenerative disease is Parkinson's disease, or early-stage Parkinson's disease, wherein the method comprises administering (e.g., orally) to a subject in need thereof a c-Abl inhibitor, preferably the c-Abl inhibitor is a compound of Formula I or a pharmaceutically acceptable salt thereof, in an amount sufficient to achieve a ratio of mean CSF Cₘₐₓ to IC₅₀ of c-Abl inhibition of from 1 to 14, wherein the subject is not concomitantly administering symptomatic medication for Parkinson's disease.

One aspect is a method for the treatment, or delay, inhibition, or suppression of the progression of, a neurodegenerative disease, preferably neurodegenerative disease is Parkinson's disease, or early-stage Parkinson's disease, wherein the method comprises administering to a subject in need thereof a c-Abl inhibitor, preferably the c-Abl inhibitor is a compound of Formula I or a pharmaceutically acceptable salt thereof, in an amount sufficient to achieve a ratio of mean CSF Cₘₐₓ to IC₅₀ of c-Abl inhibition of from 1 to 14; wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

In one embodiment of any of the methods and uses described herein, a compound of Formula I or a pharmaceutically acceptable salt thereof is administered in an amount sufficient to achieve a ratio of Cₘₐₓ to IC₅₀ greater than 5.

Yet another aspect is a method for the treatment, or delay, inhibition, or suppression of the progression of, Parkinson's disease or early-stage Parkinson's disease, wherein the method comprises administering (e.g., orally) to a subject in need thereof a compound of Formula I or a pharmaceutically acceptable salt thereof in an amount sufficient to:
a. achieve a mean plasma AUC₀₋₂₄ ranging from about 5,000 ng*h/mL to about 75,000 ng*h/mL;
b. achieve a plasma Cₘₐₓ ranging from about 500 ng/mL to about 15,000 ng/mL,
c. cross the blood-brain barrier;
d. achieve a CSF Cₘₐₓ ranging from about 0.4 ng/mL to about 7.0 ng/mL;
e. achieve a CSF concentration of at least about 0.2 ng/mL to about 2.7 ng/mL; and/or
f. achieve a ratio of mean CSF Cₘₐₓ to IC₅₀ of c-Abl inhibition of not less than 1.

Yet another aspect is a method for the treatment, or delay, inhibition, or suppression of the progression of, Parkinson's disease or early-stage Parkinson's disease, wherein the method comprises administering (e.g., orally) to a subject in need thereof a compound of Formula I or a pharmaceutically acceptable salt thereof in an amount sufficient to:
a. achieve a mean plasma AUC₀₋₂₄ ranging from about 5,000 ng*h/mL to about 75,000 ng*h/mL;
b. achieve a plasma Cₘₐₓ ranging from about 500 ng/mL to about 15,000 ng/mL,
c. cross the blood-brain barrier;
d. achieve a CSF Cₘₐₓ ranging from about 0.4 ng/mL to about 7.0 ng/mL;
e. achieve a CSF concentration of at least about 0.2 ng/mL to about 2.7 ng/mL; and/or
f. achieve a ratio of mean CSF Cₘₐₓ to IC₅₀ of c-Abl inhibition of not less than 1,
wherein the subject exhibits a disease severity according to modified Hoehn & Yahr stage≤2.

Yet another aspect is a method for the treatment, or delay, inhibition, or suppression of the progression of, Parkinson's disease or early-stage Parkinson's disease, wherein the method comprises administering (e.g., orally) to a subject in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL,
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL; and/or
f) achieve a ratio of mean CSF Cₘₐₓ to IC₅₀ of c-Abl inhibition of not less than 1,
wherein the subject exhibits a Montreal cognitive assessment score of at least 25.

Yet another aspect is a method for the treatment, or delay, inhibition, or suppression of the progression of, Parkinson's disease or early-stage Parkinson's disease, wherein the method comprises administering (e.g., orally) to a subject in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL,
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL; and/or
f) achieve a ratio of mean CSF Cₘₐₓ to IC₅₀ of c-Abl inhibition of not less than 1,
wherein the subject exhibits a Dopamine Transporter Single Photon Emission Computed Tomography (DaT SPECT) consistent with the diagnosis of Parkinson's disease.

Yet another aspect is a method for the treatment, or delay, inhibition, or suppression of the progression of, Parkinson's disease or early-stage Parkinson's disease, wherein the method comprises administering (e.g., orally) to a subject in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL,
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL; and/or
f) achieve a ratio of mean CSF Cₘₐₓ to IC₅₀ of c-Abl inhibition of not less than 1;
wherein the subject is receiving treatment with a monoamine oxidase-B (MAO-B) inhibitor.

Yet another aspect is a method for the treatment, or delay, inhibition, or suppression of the progression of, Parkinson's disease or early-stage Parkinson's disease, wherein the method comprises administering (e.g., orally) to a subject in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL,
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL; and/or
f) achieve a ratio of mean CSF Cₘₐₓ to IC₅₀ of c-Abl inhibition of not less than 1;
wherein the subject is not concomitantly administering symptomatic medication for Parkinson's disease.

Yet another aspect is a method for the treatment, or delay, inhibition, or suppression of the progression of, Parkinson's disease or early-stage Parkinson's disease, wherein the method comprises administering (e.g., orally) to a subject in need thereof a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL,
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL; and/or
f) achieve a ratio of mean CSF Cₘₐₓ to IC₅₀ of c-Abl inhibition of not less than 1,
wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Yet another aspect is a compound of Formula I or a pharmaceutically acceptable salt thereof for the preparation of a medicament, wherein the medicament comprises a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL; and/or
f) achieve a ratio of mean CSF Cₘₐₓ to IC₅₀ of c-Abl inhibition of not less than 1,
and wherein the medicament is used in the treatment, or delay, inhibition, or suppression of the progression of, Parkinson's disease or early-stage Parkinson's disease.

Yet another aspect is a compound of Formula I or a pharmaceutically acceptable salt thereof for the preparation of a medicament for the treatment, or delay, inhibition, or suppression of the progression of, Parkinson's disease or early-stage Parkinson's disease, in a subject, wherein the medicament comprises a compound of Formula I or a pharmaceutically acceptable salt thereof in an amount sufficient to:
a) achieve a mean plasma AUC₀₋₂₄ ranging from about 5,000 ng*h/mL to about 75,000 ng*h/mL;
b) achieve a plasma Cₘₐₓ ranging from about 500 ng/mL to about 15,000 ng/mL;
c) cross the blood-brain barrier;
d) achieve a CSF Cₘₐₓ ranging from about 0.4 ng/mL to about 7.0 ng/mL;
e) achieve a CSF concentration of at least about 0.2 ng/mL to about 2.7 ng/mL; and/or
f) achieve a ratio of mean CSF Cₘₐₓ to IC₅₀ of c-Abl inhibition of not less than 1;
wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Yet another aspect is a compound of Formula I or a pharmaceutically acceptable salt thereof for use in the treatment, or delay, inhibition, or suppression of the progression of, Parkinson's disease or early-stage Parkinson's disease, wherein the compound of Formula I or a pharmaceutically acceptable salt thereof safely achieves a CSF Cₘₐₓ to IC₅₀ of c-Abl inhibition ratio of not less than 1.

Yet another aspect is a compound of Formula I or a pharmaceutically acceptable salt thereof for use in the treatment, or delay, inhibition, or suppression of the progression of, Parkinson's disease or early-stage Parkinson's disease, wherein the compound of Formula I or a pharmaceutically acceptable salt thereof safely achieves a CSF Cₘₐₓ to IC₅₀ of c-Abl inhibition ratio of not less than 1;
wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to the subject in need thereof in an amount sufficient to achieve a CSF Cₘₐₓ to IC₅₀ of c-Abl inhibition ratio of not less than 1.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to the subject in need thereof in an amount sufficient to achieve a CSF Cₘₐₓ to IC₅₀ of c-Abl inhibition ratio of not less than 1, wherein the subject exhibits a disease severity according to modified Hoehn & Yahr stage≤2.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to the subject in need thereof in an amount sufficient to achieve a CSF Cₘₐₓ to IC₅₀ of c-Abl inhibition ratio of not less than 1, wherein the subject exhibits a Montreal cognitive assessment score of at least 25.

Yet another aspect is a method for reduction of the rate of progression of Parkinson's disease or early-stage Parkinson's disease, wherein the method comprises administering a compound of Formula I or a pharmaceutically acceptable salt thereof to the subject in need thereof in an amount sufficient to achieve a CSF Cₘₐₓ to IC₅₀ of c-Abl inhibition ratio of not less than 1, wherein the subject exhibits a Dopamine Transporter Single Photon Emission Computed Tomography (DaT SPECT) consistent with the diagnosis of Parkinson's disease.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to the subject in need thereof in an amount sufficient to achieve a CSF Cₘₐₓ to IC₅₀ of c-Abl inhibition ratio of not less than 1, wherein the subject is receiving treatment with a monoamine oxidase-B (MAO-B) inhibitor.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to the subject in need thereof in an amount sufficient to achieve a CSF Cₘₐₓ to IC₅₀ of c-Abl inhibition ratio of not less than 1, wherein the subject is not concomitantly administering symptomatic medication for Parkinson's disease.

Another aspect is a compound of Formula I or a pharmaceutically acceptable salt thereof for use in the treatment, or delay, inhibition, or suppression of the progression of, Parkinson's disease or early-stage Parkinson's disease, wherein the compound of Formula I or a pharmaceutically acceptable salt thereof safely achieves a CSF Cₘₐₓ to IC₅₀ of c-Abl inhibition ratio of not less than 1;
wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject exhibits a disease severity according to modified Hoehn & Yahr stage≤2.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject exhibits a Montreal cognitive assessment score of at least 25.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject exhibits a Dopamine Transporter Single Photon Emission Computed Tomography (DaT SPECT) consistent with the diagnosis of Parkinson's disease.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject is receiving treatment with a monoamine oxidase-B (MAO-B) inhibitor.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL, wherein the subject is not concomitantly administering symptomatic medication for Parkinson's disease.

Another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve a mean plasma AUC₀₋₂₄ ranging from about 5,000 ng*h/mL to about 75,000 ng*h/mL and/or a plasma Cₘₐₓ ranging from about 500 ng/mL to about 15,000 ng/mL;
wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve:
a) a CSF Cₘₐₓ ranging from about 0.4 ng/mL to about 7.0 ng/mL;
b) a CSF concentration of at least about 0.2 ng/mL to about 2.7 ng/mL; and/or
c) a ratio of mean CSF Cₘₐₓ to IC₅₀ of c-Abl inhibition of not less than 1.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve:
a) a CSF Cₘₐₓ ranging from about 0.4 ng/mL to about 7.0 ng/mL;
b) a CSF concentration of at least about 0.2 ng/mL to about 2.7 ng/mL; and/or
c) a ratio of mean CSF Cₘₐₓ to IC₅₀ of c-Abl inhibition of not less than 1,
wherein the subject exhibits a disease severity according to modified Hoehn & Yahr stage≤2.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve:
a) a CSF Cₘₐₓ ranging from about 0.4 ng/mL to about 7.0 ng/mL;
b) a CSF concentration of at least about 0.2 ng/mL to about 2.7 ng/mL; and/or
c) a ratio of mean CSF Cₘₐₓ to IC₅₀ of c-Abl inhibition of not less than 1,
wherein the subject exhibits a Montreal cognitive assessment score of at least 25.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve:
a) a CSF Cₘₐₓ ranging from about 0.4 ng/mL to about 7.0 ng/mL;
b) a CSF concentration of at least about 0.2 ng/mL to about 2.7 ng/mL; and/or
c) a ratio of mean CSF Cₘₐₓ to IC₅₀ of c-Abl inhibition of not less than 1,
wherein the subject exhibits a Dopamine Transporter Single Photon Emission Computed Tomography (DaT SPECT) consistent with the diagnosis of Parkinson's disease.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve:
a) a CSF Cₘₐₓ ranging from about 0.4 ng/mL to about 7.0 ng/mL;
b) a CSF concentration of at least about 0.2 ng/mL to about 2.7 ng/mL; and/or
c) a ratio of mean CSF Cₘₐₓ to IC₅₀ of c-Abl inhibition of not less than 1,
wherein the subject is receiving treatment with a monoamine oxidase-B (MAO-B) inhibitor.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve:
a) a CSF Cₘₐₓ ranging from about 0.4 ng/mL to about 7.0 ng/mL;
b) a CSF concentration of at least about 0.2 ng/mL to about 2.7 ng/mL; and/or
c) a ratio of mean CSF Cₘₐₓ to IC₅₀ of c-Abl inhibition of not less than 1,
wherein the subject is not concomitantly administering symptomatic medication for Parkinson's disease.

Yet another aspect is a method for reduction of the rate of progression of Parkinson's disease or early-stage Parkinson's disease, wherein the method comprises administering (e.g., orally) a compound of Formula I or a pharmaceutically acceptable salt thereof to a subject in need thereof in an amount sufficient to achieve:
a) a CSF Cₘₐₓ ranging from about 0.4 ng/mL to about 7.0 ng/mL;
b) a CSF concentration of at least about 0.2 ng/mL to about 2.7 ng/mL; and/or
c) a ratio of mean CSF Cₘₐₓ to IC₅₀ of c-Abl inhibition of not less than 1;
wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Yet another aspect is a pharmaceutical composition comprising a compound of Formula I or a pharmaceutically acceptable salt thereof for the treatment, or delay, inhibition, or suppression of the progression of, Parkinson's disease or early-stage Parkinson's disease in a subject, wherein the composition comprises the compound of Formula I or a pharmaceutically acceptable salt thereof in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1.

Yet another aspect is a pharmaceutical composition comprising a compound of Formula I or a pharmaceutically acceptable salt thereof for the treatment, or delay, inhibition, or suppression of the progression of, Parkinson's disease or early-stage Parkinson's disease in a human patient, wherein the composition comprises the compound of Formula I or a pharmaceutically acceptable salt thereof in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1,
wherein the patient exhibits a disease severity according to modified Hoehn & Yahr stage≤2.

Yet another aspect is a pharmaceutical composition comprising a compound of Formula I or a pharmaceutically acceptable salt thereof for the treatment, or delay, inhibition, or suppression of the progression of, Parkinson's disease or early-stage Parkinson's disease in a human patient, wherein the composition comprises the compound of Formula I or a pharmaceutically acceptable salt thereof in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1,
wherein the patient exhibits a Montreal cognitive assessment score of at least 25.

Yet another aspect is a pharmaceutical composition comprising a compound of Formula I or a pharmaceutically acceptable salt thereof for the treatment, or delay, inhibition, or suppression of the progression of, Parkinson's disease or early-stage Parkinson's disease in a human patient, wherein the composition comprises the compound of Formula I or a pharmaceutically acceptable salt thereof in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1,
wherein the patient exhibits a Dopamine Transporter Single Photon Emission Computed Tomography (DaT SPECT) consistent with the diagnosis of Parkinson's disease.

Yet another aspect is a pharmaceutical composition comprising a compound of Formula I or a pharmaceutically acceptable salt thereof for the treatment, or delay, inhibition, or suppression of the progression of, Parkinson's disease or early-stage Parkinson's disease in a human patient, wherein the composition comprises the compound of Formula I or a pharmaceutically acceptable salt thereof in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1,
wherein the patient is receiving treatment with a monoamine oxidase-B (MAO-B) inhibitor.

Yet another aspect is a pharmaceutical composition comprising a compound of Formula I or a pharmaceutically acceptable salt thereof for the treatment, or delay, inhibition, or suppression of the progression of, Parkinson's disease or early-stage Parkinson's disease in a human patient, wherein the composition comprises the compound of Formula I or a pharmaceutically acceptable salt thereof in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1,
wherein the patient is not concomitantly administering symptomatic medication for Parkinson's disease.

Yet another aspect is a pharmaceutical composition comprising a compound of Formula I or a pharmaceutically acceptable salt thereof for the treatment, or delay, inhibition, or suppression of the progression of, Parkinson's disease or early-stage Parkinson's disease in a human patient, wherein the composition comprises the compound of Formula I in an amount sufficient to achieve:
a) a mean plasma AUC₀₋₂₄ ranging from about 5,000 ng*h/mL to about 75,000 ng*h/mL;
b) a plasma Cₘₐₓ ranging from about 500 ng/mL to about 15,000 ng/mL;
c) a CSF Cₘₐₓ ranging from about 0.4 ng/mL to about 7.0 ng/mL;
d) a CSF concentration of at least about 0.2 ng/mL to about 2.7 ng/mL; and/or
e) a ratio of CSF Cₘₐₓ to IC₅₀ of c-Abl inhibition of not less than 1;
wherein the subject exhibits one or more (such as 2, 3, 4, 5, 6, 7, 8, 9 or 10) of:
i) a disease severity according to modified Hoehn & Yahr stage≤2;
ii) Dopamine Transporter Single Photon Emission Computed Tomography (DaT SPECT) consistent with the diagnosis of Parkinson's disease;
iii) Abnormal synuclein deposition as determined by a skin biopsy;
iv) the subject has a body mass index≥18.5 kg/m² and ≤45 kg/m²;
v) an initial diagnosis of Parkinson's disease;
vi) receiving treatment with a monoamine oxidase-B (MAO-B) inhibitor;
vii) not concomitantly administering symptomatic medication for Parkinson's disease;
viii) the subject has not received dopaminergic therapy for at least 30 days prior to the administration;
ix) diagnosed with “clinically probable Parkinson's disease” according to the MDS clinical diagnostic criteria; and
x) a Montreal cognitive assessment score of at least 25.

Another aspect of the present invention relates to a method of reducing the rate of progression of Parkinson's disease or early-stage Parkinson's disease in a human subject.

Another aspect of the present invention relates to a method of reducing the rate of progression of Parkinson's disease or early-stage Parkinson's disease in a human subject, wherein the subject exhibits a disease severity according to modified Hoehn & Yahr stage≤2.

Another aspect of the present invention relates to a method of reducing the rate of progression of Parkinson's disease or early-stage Parkinson's disease in a human subject, wherein the subject exhibits a Montreal cognitive assessment score of at least 25.

Another aspect of the present invention relates to a method of reducing the rate of progression of Parkinson's disease or early-stage Parkinson's disease in a human subject, wherein the subject exhibits a Dopamine Transporter Single Photon Emission Computed Tomography (DaT SPECT) consistent with the diagnosis of Parkinson's disease.

Another aspect of the present invention relates to a method of reducing the rate of progression of Parkinson's disease or early-stage Parkinson's disease in a human subject, wherein the subject is receiving treatment with a monoamine oxidase-B (MAO-B) inhibitor.

Another aspect of the present invention relates to a method of reducing the rate of progression of Parkinson's disease or early-stage Parkinson's disease in a human subject, wherein the subject is not concomitantly administering symptomatic medication for Parkinson's disease.

Yet another aspect of the present invention relates to a method of reducing the rate of progression of Parkinson's disease or early-stage Parkinson's disease in a human subject, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect of the present invention relates to a method of reducing the rate of progression of Parkinson's disease or early-stage Parkinson's disease in a subject (e.g., a human subject) having a disease severity according to modified Hoehn & Yahr stage≤2 comprising administering (e.g., orally) to the subject an effective amount of a compound of Formula I or a pharmaceutically acceptable salt thereof. In a preferred embodiment, the subject is a human. In another embodiment, the compound of Formula I or a pharmaceutically acceptable salt thereof is administered once a day. In another embodiment, the compound of Formula I or a pharmaceutically acceptable salt thereof is administered twice a day.

Another aspect of the present invention relates to a method of reducing the rate of progression of Parkinson's disease or early-stage Parkinson's disease in a human subject having a disease severity according to modified Hoehn & Yahr stage≤2 comprising administering (e.g., orally) to the subject an effective amount of a compound of Formula I or a pharmaceutically acceptable salt thereof once daily.

Another aspect of the present invention is a method for reducing the rate of progression of movement deficits resulting from Parkinson's disease in a human subject suffering from early-stage Parkinson's disease. Another aspect of the present invention is a method for reducing the rate of progression of movement deficits resulting from Parkinson's disease in a human subject suffering from early-stage Parkinson's disease, wherein the subject exhibits a disease severity according to modified Hoehn & Yahr stage≤2.

Another aspect of the present invention is a method for reducing the rate of progression of movement deficits resulting from Parkinson's disease in a human subject suffering from early-stage Parkinson's disease, wherein the subject exhibits a Montreal cognitive assessment score of at least 25.

Another aspect of the present invention is a method for reducing the rate of progression of movement deficits resulting from Parkinson's disease in a human subject suffering from early-stage Parkinson's disease, wherein the subject exhibits a Dopamine Transporter Single Photon Emission Computed Tomography (DaT SPECT) consistent with the diagnosis of Parkinson's disease.

Another aspect of the present invention is a method for reducing the rate of progression of movement deficits resulting from Parkinson's disease in a human subject suffering from early-stage Parkinson's disease, wherein the subject is receiving treatment with a monoamine oxidase-B (MAO-B) inhibitor.

Another aspect of the present invention is a method for reducing the rate of progression of movement deficits resulting from Parkinson's disease in a human subject suffering from early-stage Parkinson's disease, wherein the subject is not concomitantly administering symptomatic medication for Parkinson's disease.

Another aspect of the present invention is a method for reducing the rate of progression of movement deficits resulting from Parkinson's disease in a human subject suffering from early-stage Parkinson's disease, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

Another aspect of the present invention is a method for reducing the rate of progression of movement deficits resulting from Parkinson's disease in a human subject suffering from early-stage Parkinson's disease and having a disease severity according to modified Hoehn & Yahr stage≤2, the method comprising orally administering to the subject an effective amount of a compound of Formula I or a pharmaceutically acceptable salt thereof once daily.

Yet another aspect of the present invention is a method of regulating or preserving autonomic nervous function in a human subject having Parkinson's disease or early-stage Parkinson's disease. In one embodiment, the method comprises orally administering to the subject an effective amount of a compound of Formula I or a pharmaceutically acceptable salt thereof, e.g., once daily. Yet another aspect of the present invention is a method of regulating or preserving autonomic nervous function in a human subject having Parkinson's disease or early-stage Parkinson's disease, wherein the subject exhibits a disease severity according to modified Hoehn & Yahr stage≤2.

Yet another aspect of the present invention is a method of regulating or preserving autonomic nervous function in a human subject having Parkinson's disease or early-stage Parkinson's disease, wherein the subject exhibits a Montreal cognitive assessment score of at least 25.

Yet another aspect of the present invention is a method of regulating or preserving autonomic nervous function in a human subject having Parkinson's disease or early-stage Parkinson's disease, wherein the subject exhibits a Dopamine Transporter Single Photon Emission Computed Tomography (DaT SPECT) consistent with the diagnosis of Parkinson's disease.

Yet another aspect of the present invention is a method of regulating or preserving autonomic nervous function in a human subject having Parkinson's disease or early-stage Parkinson's disease, wherein the subject is receiving treatment with a monoamine oxidase-B (MAO-B) inhibitor.

Yet another aspect of the present invention is a method of regulating or preserving autonomic nervous function in a human subject having Parkinson's disease or early-stage Parkinson's disease, wherein the subject is not concomitantly administering symptomatic medication for Parkinson's disease.

Yet another aspect of the present invention is a method of regulating or preserving autonomic nervous function in a human subject having Parkinson's disease or early-stage Parkinson's disease, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

In one embodiment, the method comprises orally administering to the subject an effective amount of a compound of Formula I or a pharmaceutically acceptable salt thereof, e.g., once daily.

Yet another aspect is a method of regulating or preserving autonomic nervous function in a human subject having Parkinson's disease or early-stage Parkinson's disease and a disease severity according to modified Hoehn & Yahr stage≤2, the method comprising orally administering to the subject an effective amount of a compound of Formula I or a pharmaceutically acceptable salt thereof once daily.

Yet another aspect is a method of reducing the deterioration of autonomic nervous function due to early-stage Parkinson's disease in a human subject in need thereof. In one embodiment, the method comprises orally administering to the subject an effective amount of a compound of Formula I or a pharmaceutically acceptable salt thereof, e.g., once daily.

Yet another aspect is a method of reducing the deterioration of autonomic nervous function due to early-stage Parkinson's disease in a human subject in need thereof, wherein the subject exhibits a disease severity according to modified Hoehn & Yahr stage≤2.

Yet another aspect is a method of reducing the deterioration of autonomic nervous function due to early-stage Parkinson's disease in a human subject in need thereof, wherein the subject exhibits a Montreal cognitive assessment score of at least 25.

Yet another aspect is a method of reducing the deterioration of autonomic nervous function due to early-stage Parkinson's disease in a human subject in need thereof, wherein the subject exhibits a Dopamine Transporter Single Photon Emission Computed Tomography (DaT SPECT) consistent with the diagnosis of Parkinson's disease.

Yet another aspect is a method of reducing the deterioration of autonomic nervous function due to early-stage Parkinson's disease in a human subject in need thereof, wherein the subject is receiving treatment with a monoamine oxidase-B (MAO-B) inhibitor.

Yet another aspect is a method of reducing the deterioration of autonomic nervous function due to early-stage Parkinson's disease in a human subject in need thereof, wherein the subject is not concomitantly administering symptomatic medication for Parkinson's disease.

Another aspect of the present invention is a method of reducing the deterioration of autonomic nervous function due to Parkinson's disease or early-stage Parkinson's disease in a human subject in need thereof, wherein the subject exhibits one or more (such as 2, 3, 4, 5, 6, 7, 8, 9 or 10) of.
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

In one embodiment, the method comprises orally administering to the subject an effective amount of a compound of Formula I or a pharmaceutically acceptable salt thereof, e.g., once daily.

Yet another aspect is a method of reducing the deterioration of autonomic nervous function due to Parkinson's disease or early-stage Parkinson's disease in a human subject in need thereof and having a disease severity according to modified Hoehn & Yahr stage≤2, the method comprising orally administering to the subject an effective amount of a compound of Formula I or a pharmaceutically acceptable salt thereof once daily. Yet another aspect is a method of treating early-stage Parkinson's disease, wherein the subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

In one embodiment, the method comprises orally administering to the subject an effective amount of a compound of Formula I or a pharmaceutically acceptable salt thereof, e.g., once daily.

Yet another aspect of the present invention is a method of treating Parkinson's disease in a human subject having early-stage Parkinson's disease and a disease severity according to modified Hoehn & Yahr stage≤2, the method comprising administering (e.g., orally) to the subject an effective amount of a compound of Formula I or a pharmaceutically acceptable salt thereof once daily.

Yet another aspect is a method of treating Parkinson's disease in a human subject comprising administering (e.g., orally) once daily to the subject (i) about 192 mg or about 384 mg of a compound of Formula I or a pharmaceutically acceptable salt thereof in the form of an aqueous suspension or (ii) an amount of a compound of Formula I or a pharmaceutically acceptable salt thereof (e.g., about 134 or about 269 mg) in one or more solid dosage forms sufficient to achieve the same bioavailability as the aqueous suspension, wherein a compound of Formula I or a pharmaceutically acceptable salt thereof is orally administered in an amount sufficient to achieve (i) plasma Cₘₐₓ ranging from about 500 ng/mL to about 15,000 ng/mL, (ii) mean plasma AUC₀₋₂₄ ranging from about 5,000 ng*h/mL to about 75,000 ng*h/mL, or (iii) cerebrospinal fluid Cₘₐₓ ranging from about 0.4 ng/mL to about 7.0 ng/mL, and (iv) mean cerebrospinal fluid C_{avg} from about 0.3 ng/mL to about 4.0 ng/mL, in the subject, or any combination of (i), (ii), (iii) and (iv). In one embodiment, the human subject has a disease severity according to modified Hoehn & Yahr stage≤2. In one embodiment, the human subject exhibits a Montreal cognitive assessment score of at least 25. In one embodiment, the human subject exhibits a Dopamine Transporter Single Photon Emission Computed Tomography (DaT SPECT) consistent with the diagnosis of Parkinson's disease. In one embodiment, the human subject is receiving treatment with a monoamine oxidase-B (MAO-B) inhibitor. In one embodiment, the human subject is not concomitantly administering symptomatic medication for Parkinson's disease.

Yet another aspect of the present invention is a method of reducing the rate of progression of Parkinson's disease in a human subject comprising orally administering once daily to the subject (i) about 192 mg or about 384 mg of a compound of Formula I or a pharmaceutically acceptable salt thereof in the form of an aqueous suspension or (ii) an amount of a compound of Formula I or a pharmaceutically acceptable salt thereof (e.g., about 134 or about 269 mg) in one or more solid dosage forms sufficient to achieve the same bioavailability as the aqueous suspension or (iii) an amount of a compound of Formula I or a pharmaceutically acceptable salt thereof (e.g., about 134 or about 269 mg) in one or more solid dosage forms sufficient to achieve the same bioavailability as the aqueous suspension, wherein a compound of Formula I or a pharmaceutically acceptable salt thereof is orally administered in an amount sufficient to achieve (i) plasma Cₘₐₓ ranging from about 500 ng/mL to about 15,000 ng/mL, (ii) mean plasma AUC₀₋₂₄ ranging from about 5,000 ng*h/mL to about 75,000 ng*h/mL, or (iii) cerebrospinal fluid Cₘₐₓ ranging from about 0.4 ng/mL to about 7.0 ng/mL, and (iv) mean cerebrospinal fluid C_{avg} from about 0.3 ng/mL to about 4.0 ng/mL, or any combination of (i), (ii), (iii), and (iv) in the subject. In one embodiment, the human subject has a disease severity according to modified Hoehn & Yahr stage≤2. In one embodiment, the human subject exhibits a Montreal cognitive assessment score of at least 25. In one embodiment, the human subject exhibits a Dopamine Transporter Single Photon Emission Computed Tomography (DaT SPECT) consistent with the diagnosis of Parkinson's disease. In one embodiment, the human subject is receiving treatment with a monoamine oxidase-B (MAO-B) inhibitor. In one embodiment, the human subject is not concomitantly administering symptomatic medication for Parkinson's disease.

Yet another aspect of the present invention is a method of reducing the rate of progression of movement deficits resulting from Parkinson's disease in a human subject comprising orally administering once daily to the subject (i) about 192 mg or about 384 mg of a compound of Formula I or a pharmaceutically acceptable salt thereof in the form of an aqueous suspension or (ii) an amount of a compound of Formula I or a pharmaceutically acceptable salt thereof (e.g., about 134 or about 269 mg) in one or more solid dosage forms sufficient to achieve the same bioavailability as the aqueous suspension, wherein a compound of Formula I or a pharmaceutically acceptable salt thereof is orally administered in an amount sufficient to achieve (i) plasma Cₘₐₓ ranging from about 500 ng/mL to about 15,000 ng/mL, (ii) mean plasma AUC₀₋₂₄ ranging from about 5,000 ng*h/mL to about 75,000 ng*h/mL, or (iii) cerebrospinal fluid Cₘₐₓ ranging from about 0.4 ng/mL to about 7.0 ng/mL, and (iv) mean cerebrospinal fluid C_{avg} from about 0.3 ng/mL to about 4.0 ng/mL, or any combination of (i), (ii), (iii), and (iv) in the subject. In one embodiment, the human subject has a disease severity according to modified Hoehn & Yahr stage≤2. In one embodiment, the human subject exhibits a Montreal cognitive assessment score of at least 25. In one embodiment, the human subject exhibits a Dopamine Transporter Single Photon Emission Computed Tomography (DaT SPECT) consistent with the diagnosis of Parkinson's disease. In one embodiment, the human subject is receiving treatment with a monoamine oxidase-B (MAO-B) inhibitor. In one embodiment, the human subject is not concomitantly administering symptomatic medication for Parkinson's disease.

Yet another aspect of the present invention is a method of reducing the deterioration of autonomic nervous function due to Parkinson's disease in a human subject comprising orally administering once daily to the subject (i) about 192 mg or about 384 mg of a compound of Formula I or a pharmaceutically acceptable salt thereof in the form of an aqueous suspension or (ii) an amount of a compound of Formula I or a pharmaceutically acceptable salt thereof (e.g., about 134 or about 269 mg) in one or more solid dosage forms sufficient to achieve the same bioavailability as the aqueous suspension, wherein a compound of Formula I or a pharmaceutically acceptable salt thereof is orally administered in an amount sufficient to achieve (i) plasma Cₘₐₓ ranging from about 500 ng/mL to about 15,000 ng/mL, (ii) mean plasma AUC₀₋₂₄ ranging from about 5,000 ng*h/mL to about 75,000 ng*h/mL, or (iii) cerebrospinal fluid Cₘₐₓ ranging from about 0.4 ng/mL to about 7.0 ng/mL, and (iv) mean cerebrospinal fluid C_{avg} from about 0.3 ng/mL to about 4.0 ng/mL, or any combination of (i), (ii), (iii), and (iv) in the subject. In one embodiment, the human subject has a disease severity according to modified Hoehn & Yahr stage≤2. In one embodiment, the human subject exhibits a Montreal cognitive assessment score of at least 25. In one embodiment, the human subject exhibits a Dopamine Transporter Single Photon Emission Computed Tomography (DaT SPECT) consistent with the diagnosis of Parkinson's disease. In one embodiment, the human subject is receiving treatment with a monoamine oxidase-B (MAO-B) inhibitor. In one embodiment, the human subject is not concomitantly administering symptomatic medication for Parkinson's disease.

In one embodiment of any of the methods and uses described herein, a compound of Formula I or a pharmaceutically acceptable salt thereof is administered (e.g., orally) in an amount sufficient to achieve a Cₘₐₓ to IC₅₀ ratio of greater than about 5, such as about 6, about 7, about 8, about 9, about 10, about 11, about 12, or about 13.

In one embodiment of any of the methods and uses described herein, the subject has been diagnosed with “clinically probable Parkinson's disease” according to the MDS clinical diagnostic criteria. See, e.g., Pastuma et al., Movement Disorders, 30(12), 1591-1599, 2015. To account for multiple uses, the MDS-PD Criteria include two distinct levels of diagnostic certainty. These are:
1. Clinically Established PD: Maximizing specificity, the category is anchored with the goal that the large majority (i.e., at least 90%) will have PD. It is presumed that many true PD cases will not meet this certainty level.
2. Clinically Probable PD: Balancing sensitivity and specificity, the category is anchored with the goal that at least 80% of patients diagnosed as probable PD truly have PD, but also that 80% of true PD cases are identified.

In one embodiment of any of the methods and uses described herein, the subject has a Dopamine Transporter Single Photon Emission Computed Tomography (DaT SPECT) consistent with the diagnosis of Parkinson's disease. See, e.g., Akdemir et al., Turk. J. Med. Sci., 51(2) 400-410, 2021.

In one embodiment of any of the methods and uses described herein, the subject exhibits abnormal synuclein deposition as determined by a skin punch biopsy.

In one embodiment of any of the methods and uses described herein, administration of a compound of Formula I or a pharmaceutically acceptable salt thereof increases the time to significant worsening of the subject on the MDS-UPDRS (Unified Parkinson's Disease Rating Scale) Parts II and III.

In one embodiment of any of the methods and uses described herein, administration of a compound of Formula I or a pharmaceutically acceptable salt thereof increases the time to significant worsening of the subject on the Movement Disorder Society-Unified Parkinson's Disease Rating Scale (MDS-UPDRS) Parts I, II, and III.

In one embodiment of any of the methods and uses described herein, the c-Abl inhibitor is administered to the human subject in an amount of about 192 mg to about 384 mg.

Preferably, the c-Abl inhibitor is administered in the form of an aqueous suspension. More preferably, the c-Abl inhibitor is a compound of Formula I or a pharmaceutically acceptable salt thereof.

In one embodiment of any of the methods and uses described herein, the compound of Formula I or a pharmaceutically acceptable salt thereof is administered to the human subject in an amount of about 192 mg to about 384 mg. In one embodiment, the compound of Formula I or a pharmaceutically acceptable salt thereof is administered to the human subject in an amount of about 192 mg. In another embodiment, the compound of Formula I or a pharmaceutically acceptable salt thereof is administered to the human subject in an amount of about 384 mg. In one embodiment, the compound of Formula I or a pharmaceutically acceptable salt thereof is administered orally. In another embodiment, the compound of Formula I or a pharmaceutically acceptable salt thereof is administered in the form of an aqueous suspension. Yet in another embodiment, the compound of Formula I or a pharmaceutically acceptable salt thereof is administered in one or more dosage forms sufficient to achieve the same bioavailability as an aqueous suspension containing about 192 mg or about 384 mg of a compound of Formula I or a pharmaceutically acceptable salt thereof.

In one embodiment of any of the methods and uses described herein, the compound of Formula I or a pharmaceutically acceptable salt thereof is administered to the human subject in the range of about 48 mg to about 480 mg. In another embodiment, the compound of Formula I or a pharmaceutically acceptable salt thereof is administered to the human subject in the range of about 96 mg to about 432 mg. In another embodiment, the compound of Formula I or a pharmaceutically acceptable salt thereof is administered to the human subject in the range of about 148 mg to about 384 mg. In another embodiment, the compound of Formula I or a pharmaceutically acceptable salt thereof is administered to the human subject in the range of about 192 mg to about 336 mg. In another embodiment, the compound of Formula I or a pharmaceutically acceptable salt thereof is administered to the human subject in the range of about 240 mg to about 288 mg. In another embodiment, the compound of Formula I or a pharmaceutically acceptable salt thereof is administered to the human subject in the range of about 288 mg to about 336 mg. In another embodiment, the compound of Formula I or a pharmaceutically acceptable salt thereof is administered to the human subject in the range of about 336 mg to about 384 mg. In a preferred embodiment, the compound of Formula I or a pharmaceutically acceptable salt thereof is administered to the human subject in an amount of about 192 mg. In a more preferred embodiment, the compound of Formula I or a pharmaceutically acceptable salt thereof is administered to the human subject in an amount of about 384 mg.

In one embodiment of any of the methods described herein, the compound of Formula I or a salt thereof is administered to the human subject orally. In a more preferred embodiment, the compound of Formula I or a salt thereof is administered to the human subject in the form of an aqueous suspension. Yet in another embodiment, the compound of Formula I or a salt thereof is administered in one or more dosage forms sufficient to achieve the same bioavailability as an aqueous suspension containing about 192 mg or about 384 mg of a compound of Formula I or a pharmaceutically acceptable salt thereof.

In another embodiment, the compound of Formula I or a pharmaceutically acceptable salt thereof is administered to the human subject in an amount of about 48 mg, about 96 mg, about 144 mg, about 192 mg, about 240 mg, about 288 mg, about 336 mg, about 384 mg, or about 432 mg daily.

In one embodiment of any of the methods and uses described herein, the compound of Formula I or a pharmaceutically acceptable salt thereof is administered to the human subject once daily. In another embodiment, the compound of Formula I or a pharmaceutically acceptable salt thereof is administered to the human subject twice daily. In a preferred embodiment, the compound of Formula I or a pharmaceutically acceptable salt thereof is administered orally.

In one embodiment of any of the methods and uses described herein for the treatment of a human subject, the human subject exhibits one or more (such as 2, 3, 4, 5, 6, 7, 8, 9 or 10) of:
i) a disease severity according to modified Hoehn & Yahr stage≤2;
ii) a Dopamine Transporter Single Photon Emission Computed Tomography (DaT SPECT) consistent with the diagnosis of Parkinson's disease;
iii) abnormal synuclein deposition as determined by a skin biopsy;
iv) the subject has a body mass index≥18.5 kg/m² and ≤45 kg/m²;
v) an initial diagnosis of Parkinson's disease;
vi) the subject is receiving treatment with a monoamine oxidase-B (MAO-B) inhibitor;
vii) the subject is not concomitantly administering symptomatic medication for Parkinson's disease;
viii) the subject has not received dopaminergic therapy for at least 30 days prior to the administration;
ix) the subject has been diagnosed with “clinically probable Parkinson's disease” according to the MDS clinical diagnostic criteria; and
x) the subject exhibits a Montreal cognitive assessment score of at least 25.

The MDS-UPDRS has four parts: Part I (non-motor experiences of daily living), Part II (motor experiences of daily living), Part III (motor examination) and Part IV (motor complications). Part I has two components: IA concerns a number of behaviours that are assessed by the investigator with all pertinent information from patients and caregivers, and IB is completed by the patient with or without the aid of the caregiver, but independently of the investigator. These sections can, however, be reviewed by the rater to ensure that all questions are answered clearly and the rater can help explain any perceived ambiguities. Part II is designed to be a self-administered questionnaire but can be reviewed by the investigator to ensure completeness and clarity. Part III has instructions for the rater to give or demonstrate to the patient; it is completed by the rater.

In one embodiment of any of the methods described herein, the administration of a compound of Formula I or a pharmaceutically acceptable salt thereof slows progression of overall severity of the subject's Parkinson's disease as measured by the Clinical Global Impression Severity (CGIS) scale. The Severity of Illness is scaled as follows:
1: Normal
2: Borderline
3: Mild
4: Moderate
4: Marked
6: Severe
7: Among the Most Extremely Ill Patient

In one embodiment of any of the methods and uses described herein, the subject is not concomitantly administering symptomatic medication for Parkinson's disease.

In one embodiment of any of the methods and uses described herein, the subject is administered a compound of Formula I or a pharmaceutically acceptable salt thereof in a fasting state.

In one embodiment of any of the methods and uses described herein, the subject is administered a compound of Formula I or a pharmaceutically acceptable salt thereof in a fasting state at least 1 hour prior to administration.

In one embodiment of any of the methods and uses described herein, the subject is administered a compound of Formula I or a pharmaceutically acceptable salt thereof in a fasting state at least 2 hours prior to administration.

In one embodiment of any of the methods and uses described herein, the subject is aged 50 years or older. In another embodiment of any of the methods and uses described herein, the subject is aged 40 years or older. In one embodiment of any of the methods and uses described herein, the subject is aged 30 years or older. In one embodiment of any of the methods and uses described herein, the subject is aged 20 years or older.

In one embodiment of any of the methods and uses described herein, the subject is not concomitantly receiving dopamine replacement medication.

In one embodiment of any of the methods and uses described herein, the subject is not concomitantly administering symptomatic medication for a neurodegenerative disease (such as Parkinson's disease or early-stage Parkinson's disease).

In one embodiment of any of the methods and uses described herein, the subject has a body mass index≥18.5 kg/m² and ≤45 kg/m².

In one embodiment of any of the methods and uses described herein, the subject is administered (e.g., orally) about 192 mg or about 384 mg of a compound of Formula I or a pharmaceutically acceptable salt thereof.

In one embodiment of any of the methods and uses described herein, the subject is orally administered about 192 mg or about 384 mg of a compound of Formula I or a pharmaceutically acceptable salt thereof in the form of an aqueous suspension once daily.

In one embodiment of any of the methods and uses described herein, the subject is orally administered an amount of a compound of Formula I or a pharmaceutically acceptable salt thereof in one or more solid dosage forms sufficient to achieve the same bioavailability as an aqueous suspension containing about 192 mg or about 384 mg of a compound of Formula I or a pharmaceutically acceptable salt thereof.

In one embodiment of any of the methods and uses described herein, the subject is orally administered about 134 mg or about 269 mg of a compound of Formula I or a pharmaceutically acceptable salt thereof in the form of a solid dosage form once daily.

In one embodiment of any of the methods and uses described herein, the subject has a disease severity according to modified Hoehn & Yahr stage≤2. The modified Hoehn & Yahr scale is shown below. See, e.g., Goetz et al., Movement Disorder Society Task Force Report on the Hoehn and Yahr Staging Scale: Status and Recommendations, Mov. Disord. Off J. Mov. Disord. Soc., 19: 1020-1028, 2004; Hoehn M, Yahr M (1967). “Parkinsonism: onset, progression and mortality”. Neurology. 17 (5): 427-42: The Movement Disorder Society Task Force on Rating Scales for Parkinson's Disease”. Movement Disorders. 19 (9): 1020-1028, which are hereby incorporated by reference.

| STAGE | DEFINITION |
|---|---|
| 1.0 | Unilateral involvement only |
| 1.5 | Unilateral and axial involvement |
| 2.0 | Bilateral involvement without impairment of balance |
| 2.5 | Mild bilateral disease with recovery on pull test |
| 3.0 | Mild to moderate bilateral disease; some postural |
| | instability; physically independent |
| 4.0 | Severe disability; still able to walk or stand unassisted |
| 5.0 | Wheelchair bound or bedridden unless aided |

In one embodiment of any of the methods and uses described herein, the subject has had an initial diagnosis of Parkinson's disease within 3 years of initiating treatment with a compound of Formula I or a pharmaceutically acceptable salt thereof.

In one embodiment of any of the methods and uses described herein, the subject is not being treated with any dopamine treatment other than monoamine oxidase-B (MAO-B) inhibitor.

In one embodiment of any of the methods and uses described herein, the subject (i) is at least 50 years of age, (ii) has had an initial diagnosis of Parkinson's disease within 3 years of initiating treatment with a compound of Formula I or a pharmaceutically acceptable salt thereof, (iii) has a score on a modified Hoehn and Yahr stage of 2 or less, and (iv) is not being treated with any dopamine treatment other than monoamine oxidase-B (MAO-B) inhibitor.

In one embodiment of any of the methods and uses described herein, the subject has been receiving a stable dose of one or more monoamine oxidase B (MAOB) inhibitors for at least 10 days, preferably 20 days, more preferably 30 days.

In one embodiment of any of the methods and uses described herein, the subject has been receiving a stable dose of one or more monoamine oxidase B (MAOB) inhibitors or equivalent dose of different MAOB inhibitors for at least 10 days, preferably 20 days, more preferably 30 days.

In one embodiment of any of the methods and uses described herein, the aqueous suspension is prepared immediately prior to administration by adding a powder comprising a compound of Formula I or a pharmaceutically acceptable salt thereof to water or any water based suitable vehicle and mixing uniformly with a spoon or other stirrer.

In one embodiment of any of the methods and uses described herein, the suspension further comprises (i) polyvinyl caprolactam-polyvinyl acetate-polyethylene glycol graft co-polymer, (ii) colloidal silicon dioxide, (iii) sodium lauryl sulphate, (iv) crospovidone, (v) mannitol, and (vi) flavouring agent.

In one embodiment of any of the methods and uses described herein, a compound of Formula I is administered in free base form.

In one embodiment of any of the methods and uses described herein, the subject has been receiving treatment with a monoamine oxidase B (MAOB) inhibitors.

In one embodiment of any of the methods and uses described herein, the subject has not received dopaminergic therapy for at least 30 days prior to the administration or treatment with a compound of Formula I or a pharmaceutically acceptable salt thereof.

In one embodiment of any of the methods and uses described herein, the subject exhibits a Montreal cognitive assessment score of at least 25. See, e.g., https://www.mocatest.org.

In one embodiment of any of the methods and uses described herein, about 192 mg or about 384 mg of a compound of Formula I or a pharmaceutically acceptable salt thereof is administered to the subject.

In one embodiment of any of the methods and uses described herein, a compound of Formula I or a pharmaceutically acceptable salt thereof is administered once daily to the subject.

In one embodiment of any of the methods and uses described herein, a compound of Formula I or a pharmaceutically acceptable salt thereof is administered twice daily to the subject.

In one embodiment of any of the methods and uses described herein, a compound of Formula I or a pharmaceutically acceptable salt thereof is administered in an amount sufficient to achieve a mean plasma AUC₀₋₂₄ ranging from about 30,000 ng*h/mL to about 50,000 ng*h/mL in the subject.

In one embodiment of any of the methods and uses described herein, a compound of Formula I or a pharmaceutically acceptable salt thereof is administered in an amount sufficient to achieve a plasma Cₘₐₓ ranging from about 3,000 ng/mL to about 7,000 ng/mL.

In one embodiment of any of the methods and uses described herein, a compound of Formula I or a pharmaceutically acceptable salt thereof is administered in an amount sufficient to achieve CSF concentration of at least about 0.6 ng/mL to about 2.7 ng/mL.

In one embodiment of any of the methods and uses described herein, a compound of Formula I or a pharmaceutically acceptable salt thereof is administered in an amount sufficient to achieve CSF Cₘₐₓ ranging from about 0.4 ng/mL to about 7.0 ng/mL.

In one embodiment of any of the methods and uses described herein, the Parkinson's disease includes early-stage Parkinson's disease.

In one embodiment of any of the methods and uses described herein, upon the occurrence of an adverse event, the daily dose of a compound of Formula I or a pharmaceutically acceptable salt thereof is reduced by half.

### BRIEF DESCRIPTION OF THE DRAWINGS

FIG. 1 shows the mean concentration of vodobatinib in plasma following oral administration of 48, 192, or 384 vodobatinib to healthy human subjects.

FIG. 2 shows the mean concentration of vodobatinib in cerebrospinal fluid (CSF) following oral administration of 48, 192, or 384 vodobatinib to healthy human subjects.

### DETAILED DESCRIPTION OF THE INVENTION

### Definitions

The terms “adverse event (AE)”, “severe adverse reaction”, “severe adverse event”, “toxicity”, “treatment emergent adverse event (TEAE)” are used interchangeably and mean an event that was not present prior to the treatment of the present invention or was present at a lesser intensity and has occurred or worsened after the treatment was initiated in the patient. Such events are undesirable, unfavorable and unintended signs, symptoms or diseases that can be attributed to the use of drug or treatment. Based on the intensity and effect of adverse events the skilled person may have to modify the treatment conditions.

The terms “QTcF” and “Corrected QT interval by Fridericia” are used interchangeably and the meanings thereof is well known to the skilled person in the art.

The term “treating” or “treatment” as used herein refers to completely or partially curing, alleviating, ameliorating, improving, relieving, prevention or delaying the onset of, inhibiting progression of, reducing the severity of, and/or reducing the incidence of one or more symptoms or features of a particular disease or disease itself or disorder, and/or condition. For the purpose of present invention, the terms treating or treatment are used to refer to the treatment or prevention of a neurodegenerative disease or early-stage neurodegenerative disease. In more specific example, the term treating or treatment are used to refer treatment or prevention or amelioration of Parkinson's disease or early-stage Parkinson's disease at least one symptom thereof, in a subject.

The term “subject” as used herein refers to either a human or a non-human animal. These terms include mammals such as humans, primates, livestock animals (e.g., bovines and porcines), companion animals (e.g., canines and felines) and rodents (e.g., mice and rats).

For the purposes of the present invention, the “subject” is the one who is suffering from or has begun to show symptoms of or the one who is at the risk of development of neurodegenerative disease. In one aspect of the present invention the “subject” is the one who is suffering from or has begun to show symptoms of or the one who is at the risk of development of diseases selected from Parkinson's disease, AD or synucleinopathies such as LBD, REM sleep behaviour disorder or MSA. In one aspect of the present invention the “subject” is the one who is suffering from or has begun to show symptoms of or the one who is at the risk of development of neurodegenerative disease such as Parkinson's disease. The methods of identifying a subject are well known to the physicians or the person skilled in the art. It will be appreciated by the skilled person that some of the preliminary studies are carried out in healthy volunteers. Such healthy volunteers are also referred to as subjects for the purpose of present invention. However, the subjects are only for the demonstration purpose.

As used herein, the term “about” when appearing before a range should be understood as referring to both endpoints of the range. In such instances the range should also be understood as including the range defined by the specific endpoints listed, and also including sub-ranges within the listed endpoints. In the instances where “about’ is appearing before a number it should be understood as the number includes the range of +/−5%.

As used herein the term “between” when appearing before a range should be understood as referring to both endpoints of the range. In such instances the range should also be understood as including the range defined by the specific endpoints listed, and also including sub-ranges within the listed endpoints.

As used herein, the term “therapeutically effective amount” means an amount of a drug, for example a compound of Formula I, or a pharmaceutically acceptable salt thereof, which has a desired effect of reducing, curing or alleviating the disease or its symptoms when administered to the subject in need thereof. For the purpose of present invention, the therapeutically effective amount is effective amount of compound of Formula I, or a pharmaceutically acceptable salt thereof, that is used in the treatment, or delay, inhibition, or suppression of the progression of, a neurodegenerative disease. A therapeutically effective amount of compound of Formula I, or a pharmaceutically acceptable salt thereof, can be administered to the subject in need thereof as such or in the form of pharmaceutical formulation. In particular, the compound of Formula I, or a pharmaceutically acceptable salt thereof, is administered to a subject as a solid oral dosage form.

As used herein, the term delaying or inhibiting or suppressing the progression or reducing the rate of progression of disease such as neurodegenerative disease (e. g., Parkinson's disease or early-stage Parkinson's disease) means reducing the deterioration experienced by a subject or a patient, e.g. as quantified by one or more of UPDRS, MDS-UPDRS score, or any other method(s) as described in the present invention or a general standard method known in the art.

Early-stage Parkinson's disease or Parkinson's disease can be diagnosed by a person skilled in the field based on his experience and/or by general standard methods/techniques known in the art; e.g. Kimber T E. Approach to the patient with early Parkinson disease: diagnosis and management. Intern Med J. 2021 January; 51(1):20-26; Postuma R B, Berg D, Stern M, Poewe W, Olanow C W, Oertel W et al. MDS clinical diagnostic criteria for Parkinson's disease. Mov Disord 2015; 30: 1591-9; Hassin-Baer S et al. Identification of an early-stage Parkinson's disease neuromarker using event-related potentials, brain network analytics and machine-learning, 2022, PLoS ONE 17(1)).

An early-stage Parkinson's disease or Parkinson's disease may be identified as such by performing relevant testing or identifying symptoms by a person skilled in the art by following general standard known methods/techniques. Generally, early-stage Parkinson's disease or Parkinson's disease may be identified by one or more of the following:
a) a resting pill-rolling tremor of one hand;
b) tremor which is maximal at rest, diminishes during movement, and is absent during sleep;
c) rigidity and slowing of movement (bradycardia), decreased movement (hypokinesia), and difficulty in initiating movement (akinesia);
d) the face becoming masklike, with mouth open and diminished blinking, which may be confused with depression;
e) the posture becoming stooped;
f) difficulty in initiating waking;
g) the gait becoming shuffling with short steps, and the arms being held flexed to the waist so as to not swing with the stride;
h) steps occasionally inadvertently quickening, and the subject occasionally breaking into a run to keep from falling (festination);
i) tendency to fall forward (propulsion) or backward (retro propulsion) when the center of gravity is displaced, resulting from loss of postural reflexes;
j) speech becoming hypophonic, with a characteristic monotonous, stuttering dysarthria;
k) hypokinesia and impaired control of distal musculature resulting in micrographia and increased difficulty with daily living activities;
l) infrequent blinking and lack of facial expression;
m) decreased movement;
n) impaired postural reflexes; and
o) characteristic gait abnormality.

Pharmacokinetic (PK) evaluation is one of the endpoints of clinical trials, wherein the interaction of the subject's body with the administered substances (or drug) during the entire duration of exposure is studied mathematically. As known to the skilled person, PK evaluation includes, for example, determination of Cₘₐₓ, Tₘₐₓ, half-life, Terminal rate constant (Kel), AUC₀₋₂₄, Area under the concentration-time curve from time 0 to the last quantifiable time point (AUC₀₋ₜₐᵤ), oral clearance (CL/F), Apparent volume of distribution (V/F), and dose normalized [AUC₍₀₋ₜₐᵤ₎/dose or (Cₘₐₓ/dose)].

As used herein AUC₀₋₂₄ refers to the steady-state area under the plasma concentration versus time curve from time zero to twenty-four hours after administration of drug (e.g., a compound of Formula I or a pharmaceutically acceptable salt thereof). The plasma concentrations referred to as Cₘᵢₙ and Cₘₐₓ are the minimum and maximum steady state effective concentration of the drug in plasma during particular dosage interval, respectively. The time to reach the maximum plasma concentration after administration of the dose is referred to as Tₘₐₓ.

The terms “hour”, “hr”, “h”, “hours”, “hrs” are used interchangeably and mean unit of time.

The term “neurodegenerative disease” used herein includes but not limited to Parkinson's disease (PD), Alzheimer's disease (AD), synucleinopathies, Lewy body dementia (LBD), rapid eye movement (REM), sleep behaviour disorder, multiple system atrophy (MSA), early-stage Parkinson's disease, early stage Alzheimer's disease, early stage of synucleinopathies, early-stage lewy body dementia, early-stage rapid eye movement, early-stage sleep behaviour disorder and early-stage multiple system atrophy.

Abnormal synuclein deposition can be determined by a skin punch biopsy, such as that described in Swallow et al., Acta Neurol Scand., 130(2):59-72, 2014. Epub 2014 Apr. 5, PMID: 24702516, which is hereby incorporated by reference.

The compound of Formula I is a BCR (Breakpoint cluster region)-ABL (Abelson leukemia viral oncogene) Tyrosine Kinase Inhibitor (TKI) under investigation for the treatment of neurodegenerative disease (ND). As noted above, the compound of Formula I has been shown to be effective in the treatment of Parkinson's disease, AD and other synucleinopathies by in-vitro and in-vivo studies in mice.

In one embodiment, the present invention relates to a method for the treatment, or delay, inhibition, or suppression of the progression of, a neurodegenerative disease in a subject in need thereof. In one embodiment, a compound of Formula I or a pharmaceutically acceptable salt thereof is administered once daily to a subject in need thereof.

In one embodiment, the subject is a mammal, preferably a human.

In one embodiment, the present invention relates to a method for the treatment, or delay, inhibition, or suppression of the progression of, a neurodegenerative disease in an adult subject in need thereof. In one embodiment, a compound of Formula I or a pharmaceutically acceptable salt thereof is administered once daily to a subject in need thereof.

In one embodiment, the present invention relates to a method for the treatment, or delay, inhibition, or suppression of the progression of, Parkinson's disease (PD) in an adult subject.

In one embodiment, a compound of Formula I or a pharmaceutically acceptable salt thereof is administered once daily to a subject in need thereof.

In one embodiment, the present invention relates to a method for the treatment, or delay, inhibition, or suppression of the progression of, early-stage Parkinson's disease (PD) in an adult subject. In one embodiment, a compound of Formula I or a pharmaceutically acceptable salt thereof is administered once daily to a subject in need thereof.

In one embodiment, the present invention relates to a method for the treatment, or delay, inhibition, or suppression of the progression of, Alzheimer's disease (AD) in an adult subject. In one embodiment, a compound of Formula I or a pharmaceutically acceptable salt thereof is administered once daily to a subject in need thereof.

In one embodiment, the present invention relates to a method for the treatment, or delay, inhibition, or suppression of the progression of, Lewy body dementia (LBD) in an adult subject. In one embodiment, a compound of Formula I or a pharmaceutically acceptable salt thereof is administered once daily to a subject in need thereof.

In one embodiment, the present invention relates to a method for the treatment, or delay, inhibition, or suppression of the progression of, Rapid eye movement (REM) sleep behaviour disorder in an adult subject. In one embodiment, a compound of Formula I or a pharmaceutically acceptable salt thereof is administered once daily to a subject in need thereof.

In one embodiment, the present invention relates to a method for the treatment, or delay, inhibition, or suppression of the progression of, Multiple system atrophy (MSA) in an adult subject. In one embodiment, a compound of Formula I or a pharmaceutically acceptable salt thereof is administered once daily to a subject in need thereof.

In one embodiment, the present invention relates to a method of reducing the rate of progression of early-stage Parkinson's disease in a subject having a disease severity according to modified Hoehn & Yahr stage≤2 in a subject, preferably in an adult subject.

In one embodiment, a compound of Formula I or a pharmaceutically acceptable salt thereof is administered once daily to a subject in need thereof.

In one embodiment, the present invention relates to a method for reducing the rate of progression of movement deficits resulting from Parkinson's disease in a subject suffering from early-stage Parkinson's disease and having a disease severity according to modified Hoehn & Yahr stage≤2, preferably in an adult subject. In one embodiment, a compound of Formula I or a pharmaceutically acceptable salt thereof is administered once daily to a subject in need thereof.

In one embodiment, the present invention relates to a method of regulating or preserving autonomic nervous function in a subject having early-stage Parkinson's disease and a disease severity according to modified Hoehn & Yahr stage≤2, preferably in an adult subject. In one embodiment, a compound of Formula I or a pharmaceutically acceptable salt thereof is administered once daily to a subject in need thereof.

In one embodiment, the present invention relates to a method of reducing the deterioration of autonomic nervous function due to early-stage Parkinson's disease in a subject in need thereof and having a disease severity according to modified Hoehn & Yahr stage≤2, preferably in an adult subject. In one embodiment, a compound of Formula I or a pharmaceutically acceptable salt thereof is administered once daily to a subject in need thereof.

In one embodiment, the present invention relates to a method of treating Parkinson's disease in a subject having early-stage Parkinson's disease and a disease severity according to modified Hoehn & Yahr stage≤2, preferably in an adult subject. In one embodiment, a compound of Formula I or a pharmaceutically acceptable salt thereof is administered once daily to a subject in need thereof.

In one embodiment, the present invention relates to a method of treating Parkinson's disease in a subject having a disease severity according to modified Hoehn & Yahr stage≤2, preferably in an adult subject. In one embodiment, a compound of Formula I or a pharmaceutically acceptable salt thereof is administered once daily to a subject in need thereof.

Various techniques are available for assessing the severity and/or improvement of Parkinson's disease or symptoms thereof. In some embodiments, one or more of the following scales may be used according to the methods of the disclosure: Hoehn and Yahr staging scale; Unified Parkinson's Disease Rating Scale (UPDRS); Montreal cognitive assessment (MoCA) criteria; Clinical Global Impression Severity (CGIS); Clinical Global Impression Improvement (CGII); Clinical Global Impression Efficacy (CGIE); the Movement Disorders Society Sponsored Revision of the Unified Parkinson's Disease Rating Scale (MDS-UPDRS); Scales for Outcomes in Parkinson's Disease-motor (SCOPA-Motor); the Schwab & England Activities of Daily Living Scales (SES); the Self-assessment Parkinson's Disease Disability Scale (SPDDS); the Postural Instability and Gait Difficulty score (PIGD); Freezing of Gait Questionnaire (FOGQ); the Nonmotor Symptoms Questionnaire (NMSQuest); the Nonmotor Symptoms Scale (NMSS); Unified Dyskinesia Rating Scale (UDysRS); the Wearing-off Questionnaires (WOQ); self-reported total sleep time on the Pittsburgh Sleep quality index; the Beck Depression inventory; the Insomnia Severity Index; or combinations thereof. These techniques are well known in the art and are incorporated herein by reference in their entirety as are all publications cited herein. See, for example:
Fahn, S., Elton, R. and committee, UPDRS program members, Unified Parkinson's disease rating scale. In S. Fahn, C. Mardsen and M. Goldstein (Eds.), Recent development in a Parkinson's disease, Macmillan, New York, 1987, pp. 153-167;
Hoehn M M and Yahr M D (1967) Parkinsonism: onset, progression and mortality. Neurology 17(5): 427-442;
MoCA—Montreal cognitive assessment Test (developed by Dr. Ziad Nasreddine, 1996);
Goetz, Christopher G., et al. “MDS-UPDRS”, Movement Disorder Society, Jul. 1, 2008;
Goetz et al., Movement Disorder Society Task Force Report on the Hoehn and Yahr Staging Scale: Status and Recommendations, Mov. Disord. Off J. Mov. Disord. Soc., 19: 1020-1028, 2004; Hoehn M, Yahr M (1967). “Parkinsonism: onset, progression and mortality”. Neurology. 17 (5): 427-42: The Movement Disorder Society Task Force on Rating Scales for Parkinson's Disease”. Movement Disorders. 19 (9): 1020-1028;
Busner J, Targum S D. The clinical global impressions scale: applying a research tool in clinical practice. Psychiatry (Edgmont). 2007 July; 4(7):28-37;
CGI (Clinical Global Impression), ECDEU assessment manual, US Department of Health, Education and Welfare, p. 218-221;
Auluck P K, Chan H Y, Trojanowski J Q, Lee V M and Bonini N M (2002) Chaperone suppression of alpha-synuclein toxicity in a Drosophila model for Parkinson's disease. Science 295(5556): 865-868;
Akdemir Ü Ö, Bora Tokçaer A, Atay L Ö. Dopamine transporter SPECT imaging in Parkinson's disease and parkinsonian disorders. Turk J Med Sci. 2021 Apr. 30; 51(2):400-410;
Malek N, Swallow D, Grosset K A, Anichtchik O, Spillantini M, Grosset D G. Alpha-synuclein in peripheral tissues and body fluids as a biomarker for Parkinson's disease—a systematic review. Acta Neurol Scand. 2014 August; 130(2):59-72.

According to any one of the previous embodiments, the method of treatment comprises administering a compound of Formula I. The compound of Formula I, has the below chemical name and formula:
Chemical name: N′-(2-chloro-6-methylbenzoyl)-4-methyl-3-[2-(3-quinolyl)ethynyl]-benzohydrazide, and

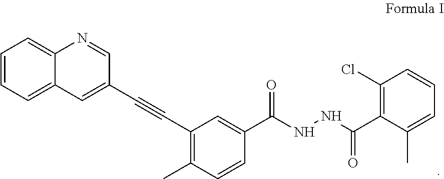 [SMILES: Cc1ccc(C(=O)NNC(=O)c2c(C)cccc2Cl)cc1C#Cc1cnc2ccccc2c1]

In an embodiment, the compound of Formula I, or a pharmaceutically acceptable salt thereof, may be administered to the subject as such. In another embodiment, the compound of Formula I, or a pharmaceutically acceptable salt thereof, may be administered as a dosage form. In a particular embodiment, the compound of Formula I, or a pharmaceutically acceptable salt thereof, may be administered as an oral dosage form.

In an embodiment, a compound of Formula I, or a pharmaceutically acceptable salt thereof, may be administered as an oral dosage form comprising the compound of Formula I, or a pharmaceutically acceptable salt thereof, in an amorphous form and a pharmaceutically acceptable excipient.

In some embodiment, the oral dosage form may be a tablet.

In some embodiments, the oral dosage form may be a hard gelatin capsule.

In some embodiments, the oral dosage form may be a sachet comprising a powder for oral suspension.

Examples of pharmaceutically acceptable excipients that may be used in the oral dosage forms of the present invention include, for example, one or more of polyvinyl caprolactam, polyvinyl acetate, polyethylene glycol graft co-polymer, silicon dioxide, sodium lauryl sulphate, silicified microcrystalline cellulose, crospovidone, gelatin, and any combination of any of the foregoing.

In one embodiment, the oral dosage form comprises (i) polyvinyl caprolactam-polyvinyl acetate-polyethylene glycol graft co-polymer, (ii) colloidal silicon dioxide, (iii) sodium lauryl sulphate, (iv) crospovidone, (v) mannitol, (vi) flavoring agent, and any combination of any of the foregoing.

In one embodiment, the oral dosage form is selected from a solid oral dosage form or an aqueous suspension.

The desired dosage form of the compound of Formula I is an oral dosage form containing from about 10 mg to about 400 mg of the compound of Formula I, or a pharmaceutically acceptable salt thereof. In an embodiment, the oral dosage form contains from about 20 mg to about 400 mg of the compound of Formula I, or a pharmaceutically acceptable salt thereof. In another embodiment, the oral dosage form contains from about 30 mg to about 400 mg of the compound of Formula I, or a pharmaceutically acceptable salt thereof. In yet another embodiment, the oral dosage form contains from about 40 mg to about 400 mg of the compound of Formula I, or a pharmaceutically acceptable salt thereof.

Generally, the daily dose may be administered over the course of one to four daily administrations. In certain embodiments, the amount of the compound of Formula I, or a pharmaceutically acceptable salt thereof, in the dosage form is between about 10 mg and about 400 mg, as measured by the amount of the compound of Formula I, or a pharmaceutically acceptable salt thereof, administered daily.

The dosage may be administered as a single daily dose.

The total daily dosage of compound of Formula I, or a pharmaceutically acceptable salt thereof, administered to a subject may be between about 10 mg to about 400 mg. In some embodiments, the total daily dosage of the compound of Formula I, or a pharmaceutically acceptable salt thereof, is about 10 mg. In some embodiments, the total daily dosage of the compound of Formula I, or a pharmaceutically acceptable salt thereof, is about 20 mg. In some embodiments, the total daily dosage of the compound of Formula I is about 24 mg. In some embodiments, the total daily dosage of the compound of Formula I, or a pharmaceutically acceptable salt thereof, is about 30 mg. In some embodiments, the total daily dosage of the compound of Formula I, or a pharmaceutically acceptable salt thereof, is about 40 mg. In some embodiments, the total daily dosage of the compound of Formula I, or a pharmaceutically acceptable salt thereof, is about 48 mg. In other embodiments, the total daily dosage of the compound of Formula I, or a pharmaceutically acceptable salt thereof, is about 96 mg. In other embodiments, the total daily dosage of the compound of Formula I, or a pharmaceutically acceptable salt thereof, is about 192 mg. In other embodiments, the total daily dosage of the compound of Formula I, or a pharmaceutically acceptable salt thereof, is about 384 mg. In some embodiments, the total daily dosage of the compound of Formula I, or a pharmaceutically acceptable salt thereof, is about 400 mg.

In an embodiment, the present invention provides a method for the treatment, or delay, inhibition, or suppression of the progression of, a neurodegenerative disease comprising administering (e.g., orally) a therapeutically effective amount of compound of Formula I, or a pharmaceutically acceptable salt thereof, to a subject in need thereof.

According to an embodiment, a therapeutically effective amount of the compound of Formula I, or a pharmaceutically acceptable salt thereof, is from about 10 mg to about 400 mg (e.g., administered orally). According to another embodiment, the therapeutically effective amount of the compound of Formula I, or a pharmaceutically acceptable salt thereof, is from about 20 mg to about 400 mg. According to yet another embodiment, the therapeutically effective amount of the compound of Formula I, or a pharmaceutically acceptable salt thereof, is from about 30 mg to about 400 mg. In one embodiment, the therapeutically effective amount of the compound of Formula I, or a pharmaceutically acceptable salt thereof, is from about 40 mg to about 400 mg. In another embodiment the therapeutically effective amount of the compound of Formula I is about 48 mg, about 192 mg or about 384 mg.

In an embodiment of the present invention, a therapeutically effective amount of a compound of Formula I, or a pharmaceutically acceptable salt thereof, is sufficient to achieve plasma PK that is effective in the treatment, or delay, inhibition, or suppression of the progression of, a neurodegenerative disease. In an embodiment, the therapeutically effective amount of the compound of Formula I, or a pharmaceutically acceptable salt thereof, is sufficient to achieve a mean plasma AUC₀₋₂₄ ranging from about 5,000 ng*h/mL to about 75,000 ng*h/mL that is effective in the treatment, or delay, inhibition, or suppression of the progression of, neurodegenerative disease. In another embodiment, the therapeutically effective amount of the compound of Formula I, or a pharmaceutically acceptable salt thereof, is sufficient to achieve a plasma Cₘₐₓ ranging from about 500 ng/mL to about 15,000 ng/mL that is effective in the treatment, or delay, inhibition, or suppression of the progression of, the neurodegenerative disease.

In another embodiment of the present invention, there is provided a method for the treatment, or delay, inhibition, or suppression of the progression of, a neurodegenerative disease wherein the method comprises administering (e.g., orally) a compound of Formula I, or a pharmaceutically acceptable salt thereof, to a subject in need thereof in an amount sufficient to achieve a mean plasma AUC₀₋₂₄ ranging from about 5,000 ng*h/mL to about 75,000 ng*h/mL.

According to another aspect, there is provided a method for the treatment, or delay, inhibition, or suppression of the progression of, a neurodegenerative disease wherein the method comprises administering (e.g., orally) a compound of Formula I, or a pharmaceutically acceptable salt thereof, to a subject in need thereof in an amount sufficient to achieve a plasma Cₘₐₓ ranging from about 500 ng/mL to about 15,000 ng/mL.

According to another aspect, there is provided a method for the treatment, or delay, inhibition, or suppression of the progression of, a neurodegenerative disease wherein the method comprises administering (e.g., orally) a compound of Formula I, or a pharmaceutically acceptable salt thereof, to a subject in need thereof in an amount sufficient to achieve a mean plasma AUC₀₋₂₄ ranging from about 5,000 ng*h/mL to about 75,000 ng*h/mL and a plasma Cₘₐₓ ranging from about 500 ng/mL to about 15,000 ng/mL.

In an embodiment of the present invention, the therapeutically effective amount of a compound of Formula I, or a pharmaceutically acceptable salt thereof, is sufficient to achieve CSF PK that is effective in the treatment, or delay, inhibition, or suppression of the progression of, a neurodegenerative disease. In an embodiment, the therapeutically effective amount of the compound of Formula I, or a pharmaceutically acceptable salt thereof, is sufficient to achieve a CSF concentration of at least about 0.2 ng/mL to about 2.7 ng/mL that is effective in the treatment, or delay, inhibition, or suppression of the progression of, a neurodegenerative disease. In another embodiment, the therapeutically effective amount of the compound of Formula I, or a pharmaceutically acceptable salt thereof, is sufficient to achieve a CSF Cₘₐₓ ranging from about 0.4 ng/mL to about 7.0 ng/mL that is effective in the treatment, or delay, inhibition, or suppression of the progression of, a neurodegenerative disease.

According to another aspect, there is provided a method for the treatment, or delay, inhibition, or suppression of the progression of, a neurodegenerative disease wherein the method comprises administering (e.g., orally) a compound of Formula I, or a pharmaceutically acceptable salt thereof, to a subject in need thereof in an amount sufficient to achieve a CSF Cₘₐₓ ranging from about 0.4 ng/mL to about 7.0 ng/mL.

According to another aspect, there is provided a method for the treatment, or delay, inhibition, or suppression of the progression of, a neurodegenerative disease wherein the method comprises administering (e.g., orally) a compound of Formula I, or a pharmaceutically acceptable salt thereof, to a subject in need thereof in an amount sufficient to achieve a CSF concentration of at least about 0.2 ng/mL to about 2.7 ng/mL.

According to yet another aspect, there is provided a method for the treatment, or delay, inhibition, or suppression of the progression of, a neurodegenerative disease wherein the method comprises administering (e.g., orally) a compound of Formula I, or a pharmaceutically acceptable salt thereof, to a subject in need thereof in an amount sufficient to achieve a CSF concentration of at least about 0.2 ng/mL.

In an embodiment, the compound of Formula I, or a pharmaceutically acceptable salt thereof, is administered (e.g., orally) in a sufficient amount to achieve a minimum concentration in the CSF of not less than about 0.5 ng/mL. In an embodiment, the compound of Formula I, or a pharmaceutically acceptable salt thereof, is administered in a sufficient amount to achieve a minimum concentration in the CSF of not less than about 0.7 ng/mL.

In an embodiment, the compound of Formula I, or a pharmaceutically acceptable salt thereof, is administered (e.g., orally) in a sufficient amount to achieve a minimum concentration in the CSF of not less than about 0.9 ng/mL. In an embodiment, the compound of Formula I, or a pharmaceutically acceptable salt thereof, is administered (e.g., orally) in a sufficient amount to achieve a minimum concentration in the CSF of not less than about 1 ng/mL. In an embodiment, the compound of Formula I, or a pharmaceutically acceptable salt thereof, is administered (e.g., orally) in a sufficient amount to achieve a minimum concentration in the CSF of not less than about 1.2 ng/mL. In an embodiment, the compound of Formula I, or a pharmaceutically acceptable salt thereof, is administered (e.g., orally) in a sufficient amount to achieve a minimum concentration in the CSF of not less than about 1.5 ng/mL. In an embodiment, the compound of Formula I, or a pharmaceutically acceptable salt thereof, is administered (e.g., orally) in a sufficient amount to achieve a minimum concentration in the CSF of not less than about 1.8 ng/mL. In an embodiment, the compound of Formula I, or a pharmaceutically acceptable salt thereof, is administered (e.g., orally) in a sufficient amount to achieve a minimum concentration in the CSF of not less than about 2 ng/mL. In an embodiment, the compound of Formula I, or a pharmaceutically acceptable salt thereof, is administered (e.g., orally) in a sufficient amount to achieve a minimum concentration in the CSF of not less than about 2.2 ng/mL. In an embodiment, the compound of Formula I, or a pharmaceutically acceptable salt thereof, is administered (e.g., orally) in a sufficient amount to achieve a minimum concentration in the CSF of not less than about 2.5 ng/mL. In an embodiment, the compound of Formula I, or a pharmaceutically acceptable salt thereof, is administered (e.g., orally) in a sufficient amount to achieve a minimum concentration in the CSF of not less than about 2.7 ng/mL.

In an embodiment, the compound of Formula I, or a pharmaceutically acceptable salt thereof, is administered in a sufficient amount to achieve a minimum concentration in the CSF of about 0.5 ng/mL to about 2.7 ng/mL. For example, the compound of Formula I, or a pharmaceutically acceptable salt thereof, is administered at a dose of about 10 mg to about 400 mg.

According to yet another aspect, there is provided a method for the treatment, or delay, inhibition, or suppression of the progression of, a neurodegenerative disease wherein the method comprises administering (e.g., orally) a compound of Formula I, or a pharmaceutically acceptable salt thereof, to a subject in need thereof in an amount sufficient to achieve a mean CSF C_{avg} of about 0.3 ng/mL to about 4.0 ng/mL.

According to yet another aspect, there is provided a method for the treatment, or delay, inhibition, or suppression of the progression of, a neurodegenerative disease wherein the method comprises administering (e.g., orally) a compound of Formula I, or a pharmaceutically acceptable salt thereof, to a subject in need thereof in an amount sufficient to achieve a CSF Cₘₐₓ ranging from about 0.4 ng/mL to about 7.0 ng/mL, a CSF concentration of at least about 0.2 ng/mL to about 2.7 ng/mL and a mean CSF C_{avg} of about 0.3 ng/mL to about 4.0 ng/mL.

According to an aspect of the present invention, there is provided a method for the treatment, or delay, inhibition, or suppression of the progression of, a neurodegenerative disease wherein the method comprises administering a c-Abl inhibitor to a subject in need thereof in an amount sufficient to achieve a ratio of mean CSF Cₘₐₓ to IC₅₀ of c-Abl inhibition of not less than 1.

The IC₅₀ for c-Abl inhibition of the compound of Formula I is 0.9 nM as compared to nilotinib, which is 20 nM when measured using a Kinase assay against native Abl (human). Thus, the compound of Formula I has been shown to be 20 times more potent than nilotinib. The compound of Formula I exhibited lower and hence potentially more effective IC₅₀ values. Further, the concentration of the compound of Formula I in CSF is equal or higher than its half-maximal inhibitory concentration ensuring optimal inhibition of c-Abl.

According to an embodiment, the methods and uses of the present invention comprise administering (e.g., orally) a compound of Formula I, or a pharmaceutically acceptable salt thereof, to a subject in need thereof in an amount sufficient to achieve a ratio of mean CSF Cₘₐₓ to IC₅₀ of c-Abl inhibition of not less than 1.

In an embodiment, the compound of Formula I, or a pharmaceutically acceptable salt thereof, is administered to a subject in need thereof in an amount sufficient to achieve a ratio of mean CSF Cₘₐₓ to IC₅₀ of c-Abl inhibition of 1. In an embodiment, the compound of Formula I, or a pharmaceutically acceptable salt thereof, is administered to a subject in need thereof in an amount sufficient to achieve a ratio of mean CSF Cₘₐₓ to IC₅₀ of c-Abl inhibition of more than 1. In an embodiment, the compound of Formula I, or a pharmaceutically acceptable salt thereof, is administered to a subject in need thereof in an amount sufficient to achieve a ratio of mean CSF Cₘₐₓ to IC₅₀ of c-Abl inhibition between about 1 to about 14. In an embodiment, the compound of Formula I, or a pharmaceutically acceptable salt thereof, is administered to a subject in need thereof in an amount sufficient to achieve a ratio of mean CSF Cₘₐₓ to IC₅₀ of c-Abl inhibition of about 14.

In an embodiment, the present invention provides a method for the treatment, or delay, inhibition, or suppression of the progression of, a neurodegenerative disease wherein the method comprises administering (e.g., orally) a c-Abl inhibitor to a subject in need thereof in an amount sufficient to:
a) cross the blood-brain barrier;
b) achieve a CSF Cₘₐₓ ranging from about 0.4 ng/mL to about 7.0 ng/mL;
c) achieve a CSF concentration at least about 0.2 ng/mL to about 2.7 ng/mL;
d) achieve a ratio of mean CSF Cₘₐₓ to IC₅₀ of c-Abl inhibition of not less than 1; or
e) any combination of a), b), c) and d)

Accordingly, in one embodiment, the c-Abl inhibitor is a compound of Formula I, or a pharmaceutically acceptable salt thereof. Further according to another embodiment, the compound of Formula I, or a pharmaceutically acceptable salt thereof is administered at a dose of about 10 mg to about 400 mg. In one embodiment, the compound of Formula I, or a pharmaceutically acceptable salt thereof is administered about 40 mg to about 400 mg.

According to a further embodiment, there is provided a method for the treatment, or delay, inhibition, or suppression of the progression of, a neurodegenerative disease wherein the method comprises orally administering to a subject in need thereof a compound of Formula I, or a pharmaceutically acceptable salt thereof, in an amount of about 40 mg to about 400 mg, wherein the compound of formula I, or a pharmaceutically acceptable salt thereof, results in a mean plasma AUC₀₋₂₄ ranging from about 5,000 ng*h/mL to about 75,000 ng*h/mL, a plasma Cₘₐₓ ranging from about 500 ng/mL to about 15,000 ng/mL, crosses the blood-brain barrier, results in a CSF Cₘₐₓ ranging from about 0.4 ng/mL to about 7.0 ng/mL and a mean CSF Cₘₐₓ of compound of Formula I is equal to or higher than the IC₅₀ of c-Abl inhibition, wherein the IC₅₀ is measured using a hAbl Kinase assay.

In an embodiment, the present invention provides a c-Abl inhibitor for the preparation of a medicament, wherein the medicament is used in the treatment, or delay, inhibition, or suppression of the progression of, a neurodegenerative disease.

According to an embodiment, the c-Abl inhibitor used for the preparation of a medicament is the compound of Formula I, or a pharmaceutically acceptable salt thereof.

According to an embodiment, the medicament is for oral or parenteral administration.

According to a particular embodiment, the medicament is for oral administration.

According to an embodiment, the medicament is a tablet, a capsule or a sachet with a powder for suspension. According to an embodiment, the medicament is a dosage form comprising a compound of Formula I, or a pharmaceutically acceptable salt thereof, and at least one excipient. According to a particular embodiment, the medicament is a dosage form comprising the compound of Formula I, or a pharmaceutically acceptable salt thereof, and one or more of excipients selected from polyvinyl caprolactam, polyvinyl acetate, polyethylene glycol graft co-polymer, silicon dioxide, sodium lauryl sulphate, silicified microcrystalline cellulose, crospovidone, gelatin and any combination of any of the foregoing.

It will be appreciated by the skilled person that the medicaments described herein can be used in the methods described herein for the treatment, or delay, inhibition, or suppression of the progression of, a neurodegenerative disease when the medicament comprises a c-Abl inhibitor, such as a compound of Formula I, or a pharmaceutically acceptable salt thereof, in an amount sufficient to achieve a mean plasma AUC₀₋₂₄ ranging from about 5,000 ng*h/mL to about 75,000 ng*h/mL; a plasma Cₘₐₓ ranging from about 500 ng/mL to about 15,000 ng/mL; crosses the blood-brain barrier; achieve a CSF Cₘₐₓ ranging from about 0.4 ng/mL to about 7.0 ng/mL; achieve a CSF concentration of at least about 0.2 ng/mL to about 2.7 ng/mL; and/or achieve a ratio of mean CSF Cₘₐₓ to IC₅₀ of c-Abl inhibition of not less than 1. In an embodiment, the amount of the compound of Formula I, or a pharmaceutically acceptable salt thereof, present in the medicament is from about 40 mg to about 400 mg.

In an aspect, the present invention provides a compound of Formula I, or a pharmaceutically acceptable salt thereof, for use in the treatment, or delay, inhibition, or suppression of the progression of, a neurodegenerative disease wherein the compound of Formula I, or a pharmaceutically acceptable salt thereof safely achieves a CSF Cₘₐₓ to IC₅₀ of c-Abl inhibition ratio of not less than 1.

The compound of Formula I, or a pharmaceutically acceptable salt thereof, is said to “safely” achieve the desired effect when it is administered to the subject in an amount that does not result in adverse events and/or cardiovascular toxicity.

The compound of Formula I, or a pharmaceutically acceptable salt thereof, when administered to both healthy volunteers and patients, in an amount sufficient to achieve a desired effect for the treatment, or delay, inhibition, or suppression of the progression of, a neurodegenerative disease did not show adverse events or cardiovascular toxicity. As can be seen from Tables 5 and 6 below, the adverse events were mild and could be controlled and reduced or eliminated with minimum care or medication. No subject experienced an SAE or a severe AE.

In vitro cardiovascular safety of the compound of Formula I has been shown in WO 2017/208267A1, page 11 onwards, and Example 5. It was also observed (study b) in the healthy volunteers that none of the subjects reported having absolute QTcF>450 ms. No AEs related to ECG parameters were reported in any group during the study showing the compound of Formula I to be safe for the heart. It was seen that even at higher concentrations in the plasma with 384 mg of compound of Formula I, the subjects did not show any major QTcF variations.

In an aspect of the present invention, there is provided a method for reduction of the rate of progression of an early-stage neurodegenerative disease, such as early-stage Parkinson's disease wherein the method comprises administering a compound of Formula I, or a pharmaceutically acceptable salt thereof, to a subject in need thereof.

According to any one of the embodiments described herein, the neurodegenerative disease is selected from Parkinson's disease, AD, or synucleinopathies such as Lewy body dementia (LBD), rapid eye movement (REM) sleep behaviour disorder or Multiple system atrophy (MSA). In one embodiment, the neurodegenerative disease is Parkinson's disease. In a particular embodiment, the present invention provides a method for the treatment, or delay, inhibition, or suppression of the progression of, Parkinson's disease (PD) in a subject in need thereof comprising administering a compound of Formula I, or a pharmaceutically acceptable salt thereof, to a subject in need thereof. In another embodiment, there is provided a method for the reduction of the rate of progression of early-stage Parkinson's disease wherein the method comprises administering a compound of Formula I, or a pharmaceutically acceptable salt thereof, to a subject in need thereof.

In one embodiment of any of the methods and uses described herein, the neurodegenerative disease includes but not limited to Parkinson's disease (PD), Alzheimer's disease (AD), synucleinopathies, Lewy body dementia (LBD), rapid eye movement (REM), sleep behaviour disorder, multiple system atrophy (MSA), early-stage Parkinson's disease, early stage Alzheimer's disease, early stage of synucleinopathies, early-stage lewy body dementia, early-stage rapid eye movement, early-stage sleep behaviour disorder and early-stage multiple system atrophy.

According to any one of the embodiments described herein, the c-Abl inhibitor includes, but is not limited to, bosutinib, saracatinib, IkT-148x, FB-101, IkT 148009, IkT 1427, SKLB 1028, imatinib, lapatinib, pazopanib, dasatinib, vandetanib, cabozantinib, axitinib, neratinib, ponatinib, lorlatinib, pexidartinib, mobocertinib, asciminib, bezuclastinib, brepocitinib, XL092, tovorafenib, dovitinib, bemcentinib, vorolanib, sbivertinib, PBI-200, RLY-4008, glumetinib, TL-895, NRD135S.E1 and ASP8273.

### EXAMPLES

### Statistical Analysis

All derivations, statistical analyses, summaries, and listings were generated using SAS® version 9.4 (SAS Institute, Inc., Cary, North Carolina). Non-compartmental PK parameter calculations were performed using Phoenix® WinNonlin® 6.4 (Certara, St. Louis, Missouri). Graphics were prepared using the same versions of SAS, or Phoenix WinNonlin, or with SigmaPlot 12.5 (Systat Software, Inc., San Jose, California).

### Example 1

### A. Inhibitory Activity of the Compound of Formula I Against A Panel of Kinases

The compound of Formula I was evaluated for its inhibitory activity on the native ABL as well as its mutant forms in kinase assays. Conventional biochemical kinase assays, using radioactivity and fluorescence as readouts, were used to derive half maximal-inhibitory concentrations (IC₅₀) values. The compound of Formula I was found to potently inhibit native ABL and its mutant forms: Abl (H396P), Abl (M351T), Abl (Q252H), Abl (Y253F), Abl (E255K), Abl (F317L), Abl (F317I) and the gatekeeper mutant Abl (T315I), as shown by the Table 1 below:

TABLE 1
In vitro Inhibitory Profile of the Compound of Formula
I Against Native and Mutant Abl Kinases:

| Kinase | IC₅₀ of the compound of Formula I (nM) |
|---|---|
| Abl | 0.9 |
| Abl (H396P) | 0.5 |
| Abl (M351T) | 0.8 |
| Abl (Q252H) | 0.8 |
| Abl (T315I) | 8 |
| Abl (Y253F) | 1 |
| Arg | 0.8 |

### Example 2

### B. To Evaluate the Safety, Tolerability and Pharmacokinetics of the Compounds of Formula I in Heathy Volunteers

An open label multiple dose study was carried out to evaluate the safety, tolerability and pharmacokinetics of the compound of Formula I in plasma and cerebrospinal fluid (CSF) in healthy volunteers. The primary objective of this study was to determine the pharmacokinetics achieved by 7 once-daily oral doses of the compound of Formula I in healthy adult male subjects. Further, it was also an object of the study to evaluate the relationship of the compound of Formula I in plasma and CSF after 7 days of once daily doses in healthy adult male subjects and to evaluate its safety and tolerability.

For this study, healthy adult non-smoker male subjects between the ages of 18 to 45 having a body mass index (BMI)≥18 Kg/m² to ≤30 Kg/m² and a total body weight >50 kg were selected. It was necessary for the subject not to show clinically significant abnormalities that would preclude lumbar puncture and/or proper collection of serial CSF samples as per the anaesthesiologist or the Principal Investigator.

The subjects were excluded if they showed inability to undergo venipuncture and/or tolerate venous access during the study or were not able to undergo lumbar puncture and tolerate continuous CSF sampling over a 24 h period or history of frequent headache, nausea or vomiting suggestive of increased intracranial pressure. Subjects with hypokalemia, hypomagnesemia or long QT syndrome or with a report of recent (6-month) alcohol abuse or illicit drug use were also excluded from the study. Further, subjects who had been on a special diet (e.g., lactose intolerance, gluten intolerance) during the 28 days prior to the first dose or who reported difficulty fasting or consuming standardized meals or subjects with a history of any relevant allergy/hypersensitivity or with history or clinical evidence of achlorhydria, severe gastrointestinal disease, particularly diarrhoea or other conditions affecting gastrointestinal mobility or absorption or syncope or fainting or a condition that predisposes them to syncope, such as, hypotension, orthostatic hypotension, bradycardia, or dehydration, or with a history or presence of significant cardiovascular, pulmonary, hepatic, gallbladder or biliary tract, renal, hematologic, gastrointestinal, endocrine, immunologic, dermatologic, neurologic, psychiatric disease, or active sexually transmitted disease were also not considered for this study. Subjects who tested positive at screening for human immunodeficiency virus (HIV), hepatitis B surface antigen (HBsAg), or hepatitis C virus (HCV) antibody, or who had donated ≥500 mL of blood within 56 days prior to the screening visit or ≥50 mL and ≤499 mL of blood within 30 days prior to the first dose, or who had participated in CSF collection studies within 56 days prior to Check-in visit were excluded from the study. Also, subjects showing screening ECG results (when repeated twice) of QTcF≥450 msec or QRS≥120 msec were excluded from the study.

Based on the above criteria, a total of 41 subjects were screened of which 19 subjects were enrolled in the study. The safety population comprised 19 subjects and the PK population comprised 18 subjects. One subject was discontinued from the study due to protocol deviation (inclusion/exclusion criteria diversion) in the 384 mg cohort. Of the 19 subjects, 6 subjects were assigned to the 48 mg cohort, 6 subjects were assigned to the 192 mg cohort and 7 subjects were assigned to the 384 mg cohort.

Healthy adult subjects included in the study underwent a complete medical history and physical examination, vital signs examination (blood pressure, pulse rate and respiratory rate, and oral temperature after sitting for at least 3 minutes), and clinical laboratory tests [chemistry, hematology, coagulation, urinalysis, human immunodeficiency virus (HIV), hepatitis B virus (HBC) and hepatitis C Virus (HCV) diagnostic profile and drug, alcohol/cotinine screen] within first 28 days of the study. Subjects had a lumbar x-ray if they did not have a report of one performed within the prior 6 months.

After 28 days of screening, the subjects were studied in sequential cohorts of 6 subjects each. The study was conducted for 17 days. There were three cohorts studied at 48 mg, 192 mg, and 384 mg of hard gelatin capsules, wherein the subject received the oral dose of the capsule every morning for 7 days after an overnight fast for at least 10 hrs. The capsules were filled with appropriate quantities of the powder blend which mainly contained melt extrudes of the compound of Formula I. Inactive ingredients in the capsules included polyvinyl caprolactam-polyvinyl acetate-polyethylene glycol graft co-polymer (Soluplus®) and auxiliary excipients such as glidants and disintegrants. After the dose, subjects continued to fast for additional 4 hours, water was allowed ad libitum with the exception of 1 hour prior and 1-hour post dose.

A pharmacokinetic (PK) assessment was conducted by measuring the plasma concentration of the compound of Formula I, wherein the blood samples (6 mL each) were collected by direct venipuncture or by use of an indwelling cannula up to 60 minutes prior to dosing and at 0.25 h, 0.5 h, 1 h, 1.5 h, 2 h, 2.5 h, 3 h, 4 h, 6 h, 8 h, 12 h and 16 h post first dose on Day 1 and after 0.25 h, 0.5 h, 1 h, 1.5 h, 2 h, 2.5 h, 3 h, 4 h, 6 h, 8 h, 12 h, 16 h and 24 h post dose on days 2 to 7.

CSF Assessment: On the morning of Day 7, local anesthetic (e.g., lidocaine 2% with epinephrine) was administered subcutaneously at the site of lumbar puncture. An indwelling intrathecal catheter was inserted at the lumbar region (L3-L5) by a trained and experienced anesthesiologist (i.e., using an epidural tray, 17G or 20/22G Tuohy needle and catheter over needle system). A CSF sample was collected for safety labs (i.e., cell count, glucose, protein). Serial CSF samples were then collected through the spinal catheter and tubing connected to a fractional collection system (i.e., peristaltic pump). The CSF samples (8 mL) were collected at a flow rate of 0.5 mL/min over a 16-minute period at pre-dose, and at 0.5 h, 1.0 h, 1.5 h, 2 h, 2.5 h, 3 h, 4 h, 6 h, 8 h, 12 h and 24 h post-dose. The first 2 mL of CSF was discarded, and the 6 mL remainder was used for the PK study.

The plasma and CSF samples were analyzed, by validated LC/MS-MS methods. PK analysis was conducted based on PK/PP Population: Cₘₐₓ, AUC₀₋ₜ, AUC₀₋₂₄, AUC_{0-∞}, Tₘₐₓ, C_{avg}, CL/F, Cₘᵢₙ, t_{1/2}, Kel, Vd (V/F; Vd/F), and Vss/F. Model-independent plasma PK parameters (Cₘₐₓ, Tₘₐₓ, t_{1/2}, AUC₀₋ₜ, AUC₀₋₂₄, AUC_{0-∞}, C_{avg}, CL/F, Vss/F, Vd/F), after the first dose (Day 1) and last dose (Day 7), Cₘᵢₙ on Days 2-7) was listed by subject and summarized by study cohort. In addition to the descriptive statistics listed above, geometric means was reported for the pivotal PK endpoints (CL/F, Vss/F, Vd/F, Cₘᵢₙ, C_{avg}, AUC₀₋ₜ, AUC₀₋₂₄, AUC_{0-∞}, and Cₘₐₓ). For CSF, PK parameters (Cₘₐₓ, Tₘₐₓ, t_{1/2}, AUC₀₋ₜ, AUC₀₋₂₄, Cₘᵢₙ, C_{avg}, CL/F, Vss/F) was listed by subject and summarized by cohort. Ratios of CSF: plasma concentration at each time point was listed by subject and summarized by study cohort.

An analysis of variance (ANOVA) model was used to investigate the effects of dose level for selected dose-normalized plasma and CSF PK parameters (Cₘₐₓ, Cₘᵢₙ, C_{avg} and AUC₀₋₂₄), across all three cohorts. The ANOVA model treated each PK parameter as the dependent variable and included dose level and day as independent variable and day dose level as interaction term.

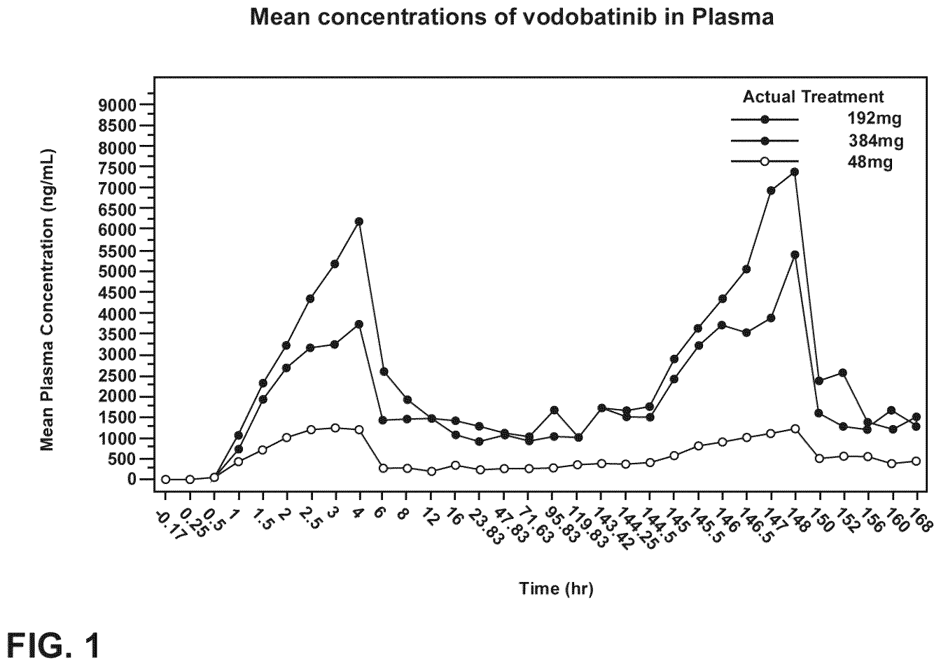

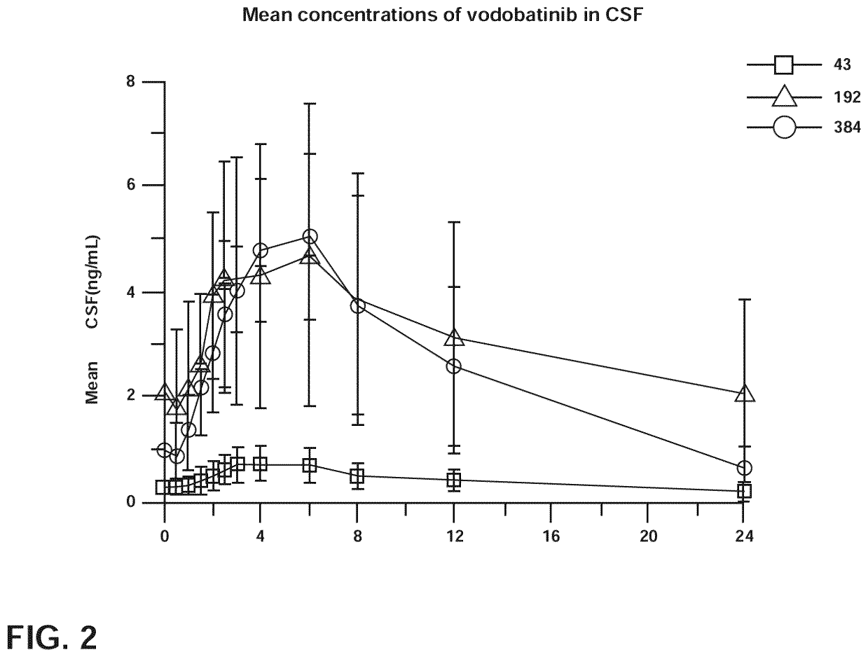

For each study cohort, descriptive statistics of the plasma and CSF concentrations of the compound of Formula I (vodobatinib) at each nominal time point, PK parameters and CSF: plasma concentration ratios were generated. Plots of mean concentration levels of plasma and CSF versus time were generated for each study cohort (see FIGS. 1 and 2).

### Results

After the initial dose on Day 1, plasma concentrations of the compound of Formula I increased with dose and the peak concentrations (Cₘₐₓ) was achieved within 2.8 hours to 4.0 hours (Table 2). Geometric mean (GM) of Cₘₐₓ values were 1334, 3551 and 5387 ng/mL for the 48 mg, 192 mg and 284 mg cohorts, respectively, with corresponding Coefficient of Variation (CV %) of 29.1%, 44.7% and 64.7%. The AUC₀₋₂₄ increased from 8692 h*ng/mL (21.0%) at 48 mg to 33426 (26.4%) at 192 mg and to 35536 (71.9%) at 384 mg.

TABLE 2
Summary of Plasma PK Parameters on Day 1 - PK Population

| | Parameter | 48 mg | 192 mg | 384 mg |
|---|---|---|---|---|
| Cₘₐₓ | n | 6 | 6 | 6 |
| (ng/mL) | AM/GM | 1378.3/1334.0 | 3775.0/3550.7 | 6631.7/5387.1 |
| | Median | 1215.0 | 3180.0 | 5580.0 |
| | Min, Max | 1010.0, 2000.0 | 2740.0, 7190.0 | 1490.0, 13700.0 |
| | SD/CV % | 400.77/29.076 | 1688.56/44.730 | 4287.98/64.659 |
| AUC₀₋₂₄ | n | 6 | 6 | 6 |
| (h*ng/mL) | AM/GM | 8870.6/8691.6 | 34474/33425.8 | 47606.3/35536.5 |
| | Median | 8883.3 | 34701.8 | 41900.5 |
| | Min, Max | 5850.1, 10810.9 | 22388.8, 45565.3 | 6644.0, 105379.2 |
| | SD/CV % | 1858.77/20.954 | 9092.76/26.376 | 34218.92/71.879 |
| Tₘₐₓ | n | 6 | 6 | 6 |
| (hour) | Median | 2.8 | 3.5 | 4.0 |
| | Min, Max | 1.50, 4.00 | 1.62, 4.00 | 1.50, 6.00 |
| | SD/CV % | 1.03/36.452 | 1.07/34.467 | 1.72/48.746 |

AM = arithmetic mean,
GM = geometric mean,
SD = standard deviation,
CV = coefficient of variation

Following once-daily doses for 7 days, trough plasma concentrations of the compound of Formula I over Days 3-7 was measured (Table 3). Mean accumulation ratios for Day 7 to Day 1 Cₘₐₓ and AUC₀₋₂₄ ranged from 1.1 to 1.6. On Day 7, the plasma exposure of the compound of Formula I increased in proportion with doses from 48 mg to 192 mg with GM (CV %) values for Cₘₐₓ as 1241 (45.7%) and 5021 (45.10%) and the value at the 384 mg dose was 6649 (68.00%). Similarly, the GM (CV %) values for AUC₀₋₂₄ were 10347 (69.7%) h*ng/mL for 48 mg, 39138 (57.5%) h*ng/mL for 192 mg and 42124 (73.7) h*ng/mL for 384 mg. Median Tₘₐₓ occurred at 4 h in the 48 mg and 192 mg cohorts and 3.5 h in the 384 mg cohort.

TABLE 3
Summary of Plasma PK Parameters on Day 7 - PK Population

| | Parameter | 48 mg | 192 mg | 384 mg |
|---|---|---|---|---|
| Cₘₐₓ | n | 6 | 6 | 6 |
| (ng/mL) | AM/GM | 1395.2/1241.4 | 5376.7/5020.9 | 7880.0/6648.7 |
| | Median | 1345.0 | 4690.0 | 6500.0 |
| | Min, Max | 421.0, 2380.0 | 3120.0, 10100.0 | 2870.0, 17900.0 |
| | SD/CV % | 638.12/45.738 | 2423.66/45.077 | 5358.67/68.003 |
| | n | 6 | 6 | 6 |
| AUC₀₋₂₄ | AM/GM | 12853.6/10346.9 | 44130.9/39138.2 | 54879.4/42124.5 |
| (h*ng/mL) | Median | 11035.5 | 40110.2 | 49986.5 |
| | Min, Max | 2972.7, 29012.6 | 20682.5, 91538.5 | 11520.9, 124799.2 |
| | SD/CV % | 8957.31/69.687 | 25396.37/57.548 | 40422.12/73.656 |
| Tₘₐₓ | n | 6 | 6 | 6 |
| (hour) | Median | 4.0 | 4.0 | 3.5 |
| | Min, Max | 1.50, 12.00 | 4.01, 4.08 | 2.52, 4.03 |
| | SD/CV % | 3.74/80.011 | 0.03/0.655 | 0.66/19.400 |

On Day 7, quantifiable CSF concentrations of the compound of Formula I were observed in all subjects in the 3 dose cohorts. After 3 to 6 hrs of dosing, the median peak CSF concentrations were 0.8 ng/mL, 5.2 ng/mL and 5.1 ng/mL at 48 mg, 192 mg and 384 mg, respectively (Table 4). The CSF GM AUC₀₋₂₄ increased from 8.9 h*ng/mL in the 48 mg cohort to 59.2 h*ng/mL and 54.5 h*ng/mL in the 192 mg and 384 mg cohorts, respectively.

TABLE 4
CSF PK Parameters

| | Parameter | 48 mg | 192 mg | 384 mg |
|---|---|---|---|---|
| Cₘₐₓ | n | 6 | 6 | 6 |
| (ng/mL) | AM/GM | 0.8/0.7 | 5.2/4.6 | 5.5/5.4 |
| | Median | 0.8 | 5.2 | 5.1 |
| | Min, Max | 0.3, 1.1 | 1.8, 8.5 | 3.7, 7.5 |
| | SD/CV % | 0.30/37.824 | 2.63/50.465 | 1.45/26.199 |
| AUC₀₋₂₄ | n | 6 | 6 | 6 |
| (h*ng/mL) | AM/GM | 10.0/8.9 | 74.5/59.2 | 58.7/54.5 |
| | Median | 10.1 | 64.5 | 60.0 |
| | Min, Max | 3.6, 15.2 | 21.1, 132.5 | 32.1, 92.1 |
| | SD/CV % | 4.80/47.990 | 50.10/67.268 | 23.47/39.992 |
| Tₘₐₓ | n | 6 | 6 | 6 |
| (hour) | Median | 3.0 | 6.0 | 5.0 |
| | Min, Max | 3.00, 6.02 | 2.03, 6.01 | 2.50, 8.01 |
| | SD/CV % | 1.22/33.176 | 1.83/37.745 | 1.96/38.558 |

The subjects were further assessed for safety. Accordingly, hematology, chemistry, urinalysis testing and vital signs examination (seated systolic [SBP] and diastolic blood pressure [DBP], pulse, respiration rate, and temperature) were performed at screening, check-in and at the exit visit prior to discharge from the study. Seated vital signs were checked after 1 h, 4 h, and 12 h of the first dose on Day 1. 12-lead ECGs were recorded on Day 1 and Day 7 in triplicate 0.75 h, 0.5 h and 0.25 h prior to the dose and after 0.5 h, 1 h, 2 h, 3 h, 4 h, 8 h, 12 h, and 24 h of the dose with subjects in supine position. Lumbar catheter was placed on day 7 and the vital signs were checked after 1 h, 4 h, and 12 h of the dose. Vital signs were examined within 15 minutes prior to the collection of samples for PK studies.

After multiple once-daily doses for 7 days, concentrations of the compound of Formula I in plasma and CSF showed dose-related increases from 48 mg to 192 mg with a less than proportional increase at 384 mg. With once-daily dosing for 7 days, over the 3 cohorts the increase in the mean range of plasma accumulation of the compound of Formula I was from 1.1 fold to 1.6 fold.

The safety and tolerability of the subjects treated with the compound of Formula I was assessed by the incidence of treatment emergent AEs, study discontinuation information, laboratory test results, vital signs, serial ECGs, CSF safety labs, CSF tip culture and physical examination findings. All adverse event (AE)s were captured and documented throughout the study from the time of ICF signing to 7 days after the last dose of study medication or early termination.

The AEs were coded according to MedDRA® and grouped by Preferred Terms (PT) and System Organ Class (SOC). All AEs were classified and graded according to CTCAE Version 4.03 or later. Incidence (n) and percentage (%) was reported for coded TEAEs by cohort for the SAF Population. Subjects with more than one of the same PTs within an SOC were counted only once.

### Results

Eleven subjects (58%) reported 28 TEAEs. Four subjects reported 7 treatment-related TEAEs. TEAEs unlikely related and unrelated to study treatment were reported in 21% and 37% subjects, respectively. The TEAEs were mild (58% subjects) to moderate (21% subjects) in intensity and none were more intense. There were no SAEs. No fatal or unknown outcomes were reported.

Only one subject reported one TEAE which had not resolved by the end of the study. Overall, no clinically relevant or significant findings were observed related to clinical laboratory results, vital signs, ECGs, or physical examinations. A brief summary of adverse events reported is presented in Table 5.

TABLE 5
Brief summary of Adverse Events:

| | 48 mg | | 192 mg | | 384 mg | | Total | |
|---|---|---|---|---|---|---|---|---|
| | (N = 6) | | (N = 6) | | (N = 7) | | (N = 19) | |
| Number of subjects with | 4 | (66.7) | 4 | (66.7) | 3 | (42.9) | 11 | (57.9) |
| at least one TEAE | | | | | | | | |
| Number of TEAEs | 9 | | 10 | | 9 | | 28 | |
| Study Drug Related | 1 | (16.7) | 1 | (16.7) | 2 | (28.6) | 4 | (21.1) |
| TEAEs | | | | | | | | |
| Mild | 4 | (66.7) | 4 | (66.7) | 3 | (42.9) | 11 | (57.9) |
| Moderate | 1 | (16.7) | 2 | (33.3) | 1 | (14.3) | 4 | (21.1) |
| Severe | — | | — | | — | | — | |
| Unrelated | 4 | (66.7) | 3 | (50.0) | 0 | | 7 | (36.8) |
| Unlikely | 0 | | 1 | (16.7) | 3 | (42.9) | 4 | (21.1) |
| Possibly | 1 | (16.7) | 1 | (16.7) | 2 | (28.6) | 4 | (21.1) |
| Probably | 0 | | 0 | | 1 | (14.3) | 1 | (5.3) |
| Certainly | 0 | | 0 | | 0 | | 0 | |
| None | 4 | (66.7) | 4 | (66.7) | 3 | (42.9) | 11 | (57.9) |

| | | | | |
|---|---|---|---|---|
| Study Drug Withdrawn | 0 | 0 | 0 | 0 |

The TEAs by SOC and PT that were reported and related to drug are presented in Table 6. Four subjects reported 7 treatment-related TEAEs (one subject in 48 mg, one subject in 192 mg and 2 subjects in 384 mg dose groups). These were: headache in one subject (48 mg); headache, nausea, and myalgia in one subject (192 mg); and nausea and abdominal distention in one subject, and headache in one subject (384 mg). These events were mild in intensity and resolved at the end of the study. The study drug related TEAs are presented in Table 6.

TABLE 6
Incidence of IMP Related TEAEs by SOC and PT; N (%)

| System Organ Class | 48 mg | 192 mg | 384 mg | Total | |
|---|---|---|---|---|---|
| Preferred Term | (N = 6) | (N = 6) | (N = 7) | (N = 19) | |
| Subjects with at least one AE | 1 (16.7) | 1 (16.7) | 2 (28.6) | 4 | (21.1) |
| Subjects with more than one AE | 0 | 1 (16.7) | 1 (14.3) | 2 | (10.5) |
| Gastrointestinal Disorders | 0 | 1 (16.7) | 1 (14.3) | 2 | (10.5) |
| Nausea | 0 | 1 (16.7) | 1 (14.3) | 2 | (10.5) |
| Abdominal Distension | 0 | 0 | 1 (14.3) | 1 | (5.3) |
| General Disorders and Administration | 0 | 0 | 1 (14.3) | 1 | (5.3) |
| Site Conditions | | | | | |
| Pain | 0 | 0 | 1 (14.3) | 1 | (5.3) |
| Musculoskeletal and Connective | 0 | 1 (16.7) | 0 | 1 | (5.3) |
| Tissue Disorders | | | | | |
| Myalgia | 0 | 1 (16.7) | 0 | 1 | (5.3) |
| Nervous System Disorders | 1 (16.7) | 1 (16.7) | 1 (14.3) | 3 | (15.8) |
| Headache | 1 (16.7) | 1 (16.7) | 1 (14.3) | 3 | (15.8) |

Categorical ECG analysis included the number and percentage of individuals with abnormal ECG findings and those with: absolute QTcF values >450 milliseconds, absolute QTcF values >480 milliseconds, absolute QTcF values >500 milliseconds, change from baseline >30 msec, and change from baseline >60 msec. No subject reported clinically significant abnormalities. None of the subjects reported having absolute QTcF>450 ms. No AEs related to ECG parameters were reported in any group during the study.

The compound of Formula I was safe and generally well tolerated by the healthy volunteers at 48 mg, 192 mg and 384 mg doses.

### Example 3

c. To Evaluate the Safety, Tolerability and Pharmacokinetics of the Compound of Formula I in Parkinson's Disease Patients

In this study, a total of 60 subjects with Parkinson's disease were screened in the double-blind part of the study, and 48 subjects were enrolled in 6 treatment dose cohorts of: 6 mg, 12 mg, 18 mg, 24 mg, 36 mg, 192 mg, and 384 mg. Of these, 44 subjects completed the study. An additional 10 subjects were enrolled in the open label part of the study, of these, one subject was discontinued due to an AE of vomiting.

Safety population was comprised of 48 subjects (not including those in the open label cohorts) and PK population was comprised of 36 subjects from double blind part and 10 subjects from open label part of the study. No subjects were discontinued from the study due to protocol deviation.

The PK analysis was performed on the PK population and safety analysis was performed on this population as per the protocol described before.

Plasma concentrations of the compound of Formula I increased with dose (see Table 7). Mean Cₘₐₓ was 182 ng/mL at 6 mg up to 4512 ng/mL at 384 mg. Similarly, AUC₀₋₂₄ increased with dose ranging from 1250 h*ng/mL at 6 mg to 43821 h*ng/mL in the 384 mg cohort. With once-daily dosing, plasma accumulation ranged from 0.94 up to 3.5-fold. In the few subjects who were enrolled in the open-label portion of the study and received the 24 mg, 48 mg or 96 mg once-daily doses, plasma concentrations of the compound of Formula I in these subjects were comparable to those in the double-blind portion of the study. CSF concentrations of the compound of Formula I collected at 2 h-3 h post-dose ranged from below limit of quantification (BLQ) (<0.05 ng/mL) to 1.70 ng/mL.

TABLE 7
Summary of the Compound of Formula I Plasma PK Parameters by Dose Cohort

| | | | | | | | | |
|---|---|---|---|---|---|---|---|---|
| PK | | | | | | | | |
| Parameter | 6 mg | 12 mg | 18 mg | 24 mg | 48 mg | 96 mg | 192 mg | 384 mg |
| (unit) | (N = 6) | (N = 6) | (N = 5) | (N = 9) | (N = 9) | (N = 10) | (N = 6) | (N = 6) |

Max Conc (ng/mL)

| | | | | | | | | |
|---|---|---|---|---|---|---|---|---|
| n | 6 | 6 | 4 | 8 | 9 | 10 | 5 | 5 |
| Mean (SD) | 181.67 | 301.00 | 308.50 | 715.25 | 1110.44 | 2802.50 | 3233.80 | 4512.00 |
| | (171.538) | (171.415) | (81.361) | (261.662) | (630.976) | (1795.605) | (2582.363) | (1623.197) |
| Median | 133.50 | 242.50 | 310.00 | 657.00 | 844.00 | 2350.00 | 2580.00 | 5320.00 |
| Min, Max | 67.3, | 170.0, | 212.0, | 457.0, | 345.0, | 645.0, | 899.0, | 1930.0, |
| | 523.0 | 628.0 | 402.0 | 1110.0 | 2060.0 | 6560.0 | 7650.0 | 5950.0 |
| Geo. Mean | 138.72 | 270.17 | 300.12 | 677.41 | 930.30 | 2298.86 | 2561.39 | 4198.52 |
| CV (%) | 94.42 | 56.95 | 26.37 | 36.58 | 56.82 | 64.07 | 79.86 | 35.98 |

AUC Over Dosing Interval (h*ug/L)

| | | | | | | | | |
|---|---|---|---|---|---|---|---|---|
| n | 6 | 6 | 4 | 8 | 9 | 10 | 5 | 5 |
| Mean (SD) | 1249.59 | 3249.33 | 3371.15 | 5723.61 | 7911.46 | 17129.39 | 33788.39 | 43820.79 |
| | (695.947) | (2512.785) | (1642.777) | (2273.764) | (3703.298) | (9801.103) | (27707.344) | (17739.540) |
| Median | 1042.37 | 2533.78 | 4158.50 | 6642.14 | 8666.88 | 13906.34 | 24800.20 | 44514.41 |
| Min, Max | 470.4, | 1441.8, | 908.4, | 2455.8, | 2906.0, | 6865.7, | 13784.8, | 18638.0, |
| | 2361.4 | 8242.2 | 4259.3 | 8084.7 | 14247.2 | 38709.4 | 82676.0 | 66662.2 |
| Geo. Mean | 1092.98 | 2713.48 | 2859.95 | 5250.28 | 700.527 | 15116.35 | 27607.37 | 40386.32 |
| | 55.69 | 77.33 | 48.73 | 39.73 | 46.81 | 57.22 | 82.00 | 40.48 |

Half-Life Lambda Z (h)

| | | | | | | | | |
|---|---|---|---|---|---|---|---|---|
| n | 1 | 0 | 1 | 2 | 3 | 4 | 0 | 0 |
| Mean (SD) | 11.13 | | 13.46 | 10.64 | 19.62 | 15.67 | | |
| | | | | (2.469) | (14.484) | (8.327) | | |
| Median | 11.13 | | 13.46 | 10.64 | 11.35 | 15.46 | | |
| Min, Max | 11.1, | | 13.5, | 8.9, | 11.2, | 5.7, | | |
| | 11.1 | | 13.5 | 12.4 | 36.3 | 26.1 | | |
| Geo. Mean | 11.13 | | 1346 | 10.50 | 16.64 | 13.22 | | |
| CV (%) | | | | 23.21 | 73.82 | 53.15 | | |

Time of Cₘₐₓ (h)

| | | | | | | | | |
|---|---|---|---|---|---|---|---|---|
| n | 6 | 6 | 4 | 8 | 9 | 10 | 5 | 5 |
| Mean (SD) | 2.34 | 1.64 | 2.48 | 2.28 | 2.89 | 2.31 | 1.50 | 3.76 |
| | (0.815) | (0.617) | (1.893) | (1.138) | (2.036) | (1.333) | (1.579) | (3.899 |
| Median | 2.00 | 2.00 | 2.96 | 2.00 | 2.02 | 2.00 | 1.00 | 1.77 |
| Min, Max | 2.0, 4.0 | 0.6, 1 | 0.0, 4.0 | 0.5, 4.0 | 1.8, 8.0 | 0.0, 4.0 | 0.0, 4.0 | 0.0, 8.0 |
| Geo. Mean | 2.25 | 1.50 | 3.15 | 1.97 | 2.51 | 2.35 | 1.43 | 3.32 |
| CV (%) | 34.89 | 37.74 | 76.37 | 49.98 | 70.46 | 57.74 | 105.00 | 103.70 |

[symbol]

[symbol]

indicates data missing or illegible when filed

An analysis of the compound of Formula I in CSF in a patient was carried out in a few subjects in the open-label portion of the study who received 24 mg, 48 mg or 96 mg doses for 14 days. A single CSF sample was collected at either 2 h or 3 h following the dose on Day 14. CSF concentrations of the compound of Formula I ranged from BLQ (<0.05 ng/mL) to 1.70 ng/ml (Table 8).

TABLE 8
Summary of the Concentration (ng/mL) of the compound
of Formula I in CSF for Open Label Part - Day 14

| PK Parameter | | | |
|---|---|---|---|
| (unit) | 24 mg (N = 3) | 48 mg (N = 3) | 96 mg (N = 4) |
| n | 2 | 3 | 4 |
| Mean (SD) | 0.6550 (0.24466) | 0.6683 (0.74402) | 1.1193 (0.45331) |
| Median | 0.6550 | 0.5350 | 1.0850 |
| Min, Max | 0.482, 0.828 | 0.000, 1.470 | 0.607, 1.700 |
| Geo. Mean | 0.6317 | 0.8868 | 1.0482 |
| CV % | 37.3525 | 111.3239 | 40.5011 |

### Safety Study Results:

Overall, a total of 32 subjects reported 63 TEAEs. Twenty-one subjects reported 35 treatment related TEAEs.

Twenty-three subjects reported 42 TEAEs in the double-blind part of the study: one subject each in the 24 mg and 48 mg cohorts; 2 subjects each in the 18 mg and 96 mg cohorts; 3 subjects each in the 6 mg, 12 mg, and 192 mg cohorts; and 5 subjects in the 384 mg cohort.

Nine subjects reported 21 TEAEs in the open-label part of the study: 3 subjects each reported 10, 6, and 5 TEAEs in the 24 mg, 48 mg, and 96 mg cohorts, respectively.

Most common TEAEs (>1 subject) in the double-blind part of the study were: abdominal pain reported in the 96 mg, 192 mg, and 384 mg cohorts, blood urine presence was reported in the 24 mg and placebo cohorts, rash was seen in the 6 mg, 192 mg and 384 mg cohorts, headache in the 6 mg and 384 mg cohorts, and tremor in the 6 mg and placebo cohorts. Most common treatment related TEAEs occurring in (>1 subject) were: rash, abdominal pain and tremor. These were of grade 1 severity except two events—abdominal pain of grade 2 severity in one subject and rash of grade 2 severity in one subject. Most common TEAEs occurring in (>1 subject) in the open-label part of the study were: headache (24 mg and 48 mg cohorts) and asthenia (24 mg and 48 mg cohorts). Treatment related TEAEs were asthenia, vertigo, vomiting and nausea (one subject in the 24 mg cohort); headache (two events in two subjects in the 24 mg cohort and one event in one subject in the 48 mg cohort); dry eye and muscle spasm (one subject each in the 96 mg cohort).

Severity of TEAEs was seen in one subject who reported treatment unrelated asthenia of grade 3 severity in the 48 mg group, and the event was resolved. All other TEAEs were of grade 1 to grade 2 intensity.

Three subjects were discontinued from the study due to AEs (the double-blind part of the study: rash/pruritus [the 192 mg cohort] and abdominal pain [the 384 mg cohort]; and the open-label part of the study: vomiting [the 24 mg cohort]).

There were no drug-related changes in the QT interval. There were no clinically significant drug related changes in laboratory values.

Overall the compound of Formula I demonstrated acceptable safety and tolerability in the study along with considerable exposure in Plasma and CSF.

### Example 4

### In Vitro Studies

c-Abl Kinase Assay

Inhibition of c-Abl kinase activity by vodobatinib was determined in a radiometric detection assay using γ-33P-labeled-ATP. A 50× working stock solution of test compound in DMSO was spiked into a kinase mixture (final DMSO concentration 2%) comprising recombinant human c-Abl, 8 mM MOPS pH 7.0, 0.2 mM EDTA, 50 μM EAIYAAPFAKKK (Abl substrate peptide), 10 mM magnesium acetate, and [γ-33P-ATP]. The reaction was initiated by addition of an MgATP mix. Following incubation for 40 minutes at room temperature, the reaction was stopped by addition of 3% phosphoric acid solution. The radiolabeled substrate was separated onto a P30 filtermat, which was washed with 75 mM phosphoric acid and methanol, and dried, following which radioactivity incorporated into the substrate was determined by scintillation counting. Similar assessments were also carried out using recombinant murine c-Abl enzyme using the identical assay format as described above.

### Binding Affinity Determination

The binding affinity of vodobatinib to c-Abl was determined in a competition binding KINOMETM scan assay. Streptavidin-coated magnetic beads were treated with biotinylated ligand (ATP) for 30 minutes to generate affinity resins for kinase assays. Liganded beads were blocked with excess biotin, and washed with blocking buffer (SEA BLOCK Pierce), with 1% BSA, 0.05% Tween 20, 1 mM DTT) to remove unbound ligand and reduce non-specific binding. For assay reactions, DNA-tagged c-Abl kinase, immobilized ligand (ATP) on streptavidin-coated affinity magnetic beads, and vodobatinib were incubated together in binding buffer (20% SEA BLOCK, 0.17×PBS, 0.05% Tween 20, 6 mM DTT) in polystyrene 96-well plates at a final volume of 0.135 mL. Following shaking incubation at room temperature for 1 h, the affinity beads were washed with 1×PBS, 0.05% Tween 20. The beads were then re-suspended in elution buffer (1×PBS, 0.05% Tween 20, 0.5 M non-biotinylated affinity ligand) and incubated at room temperature with shaking for 30 min. The kinase concentration in the eluates was measured by qPCR. The kinase concentrations in test compound treated wells were compared with that in the non-treated wells across the concentration range to generate the Kd values.

### Example 5

### In Vivo Studies

### Plasma and CSF Pharmacokinetics of Vodobatinib in Healthy Volunteers

Forty-one healthy adult male volunteers aged 18 to 45 with body mass indexes (BMI)≥18 to ≤30 Kg/m² and total body weight >50 kg (110 lb) were screened in order to enroll 19 subjects with an average age of 34.2 years (range 21.5-45.6 years) into this study Six participants were randomly assigned to the 48 and 192 mg cohorts, and seven participants were assigned to the 384 mg cohort. After an overnight fast, participants received once daily doses of vodobatinib as capsules for 7 days. Serial PK blood samples to measure vodobatinib plasma concentrations were collected at pre-dose and at 0.25, 0.5, 1, 1.5, 2, 2.5, 3, 4, 6, 8, 12, 16 and 24 h post-dose after the first dose on Day 1 and the last dose on day 7. Additional PK samples prior to the morning doses were taken on Days 3-6. On the morning of Day 7, prior to dosing, an indwelling intrathecal catheter was inserted in the lumbar region (L3-L5). Serial CSF samples were collected through the catheter connected via peristaltic pump to a fraction collector. At each designated CSF collection time point, 8 mL of CSF was collected at a flow rate of 0.5 mL/min over a 16-min period at pre-dose, and 0.5, 1.0, 1.5, 2, 2.5, 3, 4, 6, 8, 12 and 24 h post-dose. The first 2 mL of CSF was discarded, and the remaining 6 mL was used for the vodobatinib assay. The occurrence of treatment-emergent adverse events (TEAEs) and serious AEs (SAEs) reported through the completion of study were collected. Clinical laboratory parameters, vital signs and physical examination data and electrocardiogram (ECG) were collected and evaluated relative to baseline values.

### Pk, Safety and Tolerability in Parkinson's Disease Patients

Study NCT02970019 was a multi-center safety and tolerability study of vodobatinib in 60 subjects with Parkinson's Disease (diagnosed with PD according to the Brain Bank criteria of the United Kingdom Parkinson's Disease Society) who were randomized to receive placebo or various daily doses of vodobatinib for 14 days in a parallel assignment design with quadruple masking (participant, care provider, investigator, and outcomes assessor.) In study NCT02970019, consecutive cohorts of up to eight subjects received once-daily doses of 6, 12, 18, 24, 48, 96, 192, or 384 mg of study drug for 14 days, with six subjects in each cohort on vodobatinib and two subjects in each cohort on placebo. An alternating panel design was used such that subjects in the 6 and 18 mg cohorts were the same, and subjects in the 12 and 24 mg cohorts were the same, thus yielding 48 total individuals enrolled in the eight cohorts. There was a wash-out period of 14 days between each were studied in sequential ascending dose cohorts. In parallel to the double-blind cohorts, up to four subjects per dose cohort were enrolled in an open-label study of vodobatinib to receive 24, 48 or 96 mg once-daily doses of for 14 days. These subjects underwent lumbar puncture to collect CSF on Day 1, and 2-3 h after the last morning dose on Day 14. PK blood draws (for both double-blind and open label) were collected at pre-dose (˜30 min) and at 0.5, 1, 2, 4, 8, 12 and 24 h post-dose on Days 1 and 14.

### Bioanalysis and PK Assessments

Vodobatinib levels in plasma and CSF were determined using a fully validated LC-MS/MS assay with lower limits of quantitation (LLOQ) of 2.5 ng/mL and 0.05 ng/mL, respectively. PK analyses were performed using standard non-compartmental analysis with Phoenix® WinNonlin® software Version 6.4 to estimate the PK parameters Cₘₐₓ, Tₘₐₓ, AUCₜₐᵤ and Css. Descriptive statistics were used to characterize vodobatinib concentrations and PK parameters in plasma and CSF. Statistical calculations were performed with SAS software (Version 9.4, SAS Institute, Cary, North Carolina, USA).

### In Vitro Studies

In the functional radiometric enzyme kinase assay, vodobatinib inhibited c-Abl with an IC₅₀ of 0.9 nM and was thus 16-50 fold more potent than the reported nilotinib IC50 of 15-45 nM in this respect (Table 9). See, e.g., D. K. Walters et al., Cancer Res., 65 (11), 4500-4505, 2005.

TABLE 9
Potency of Vodobatinib and Nilotinib in in vitro Assays vs. Human Abl Enzyme

| | In Vitro Kinase Inhibition IC₅₀ nM | |
|---|---|---|
| | Vodobatinib | Nilotinib |
| hAbl Kinase Assay (substrate phosphorylation) | 0.9 | 15 ^{A} |
| | | 45 ^{B} |
| Binding Assay (K_{d} nM); DiscoverX) − hAbl | 0.98 | |
| hAbl Autophosphorylation in Ba/F3 Cells | | 20 ± 2 ^{C, D} |
| | | 17 ± 0.5 ^{G} |
| | | 21 ± 2 ^{E, F} |
| hAbl Autophosphorylation in K562 Cells | | 43 ± 15 ^{D} |
| HAbl Autophosphorylation in KU-812 Cells | | 60 ± 19 ^{D} |

^{A} T. O’Hare, D. K. Walters, E.P. Stoffregen, T. Jia, P. W. Manley, J. Mestan, S. W. Cowan-Jacob, F. Y. Lee, M. C. Heinrich, M. W. N. Deininger, B. J. Druker, In vitro activity of Bcr-Abl inhibitors AMN107 and BMS-354825 against clinically relevant imatinib-resistant Abl kinase domain mutants, Cancer Res. 65 (11) (2005 Jun. 1) 4500-4505.
^{B} U. Rix, O. Hantschel, G. Durnberger, L. L. Remsing, M. Planyavsky, N. V. Fernbach, I. Kaupe, K. L. Bennett, P. Valent, J. Colinge, T. Kocher, G. Superti-Furga, Chemical proteomic profiles of the BCR-ABL inhibitors imatinib, nilotinib, and dasatinib reveal novel kinase and nonkinase targets, Blood 110 (12) (2021) 4055-4063.
^{C} E. Weisberg, P. Manley, J. Mestan, S. Cowan-Jacob, A. Ray,J. D. Griffin, AMN107 (nilotinib): a novel and selective inhibitor of BCR-ABL, J. Cancer 94 (12) (2006 Jun. 19) 1765-1769.
^{D} Tasigna (Nilotinib, Oral capsules. Application number 22-068, summary basis of approval, USFDA, CDER https://www.accessdata.fda.gov/drugsatfda_docs/nda/2007/022068s000_PharmR_P1.pdf. (Accessed 27 May 2022),
^{E} P. W. Manley, P. Drueckes, G. Fendrich, P. Furet, J. Liebetanz, G. Martiny-Baron, J. Mestan, J. Trappe, M. Wartmann, D. Fabbro, Extended kinase profile and properties of the protein kinase inhibitor nilotinib, Biochem. Biophys. Acta 1804 (3) (2010 March) 445-453.
^{F} E. Weisberg, P. Manley, W. Breitenstein, J Bruggen, S. W. Cowan-Jacob, A. Ray, B. Huntly, D. Fabbro, G. Fendrich, E. Hall-Meyers, A. L. Kung, J. Mestan, G. Q. Daley, Callahan, L. Catley, C. Cavazza, M. Azam, D. Neuberg, R. D. Wright, D. Gilliland, J. D. Griffin, Characterization of AMN107, a selective inhibitor of native and mutant, Bcr-Abl Cancer Cell 7 (2) (2005 February) 129-141.
^{G} P. W. Manley, S. W. Cowan-Jacob, G. Fendrich, J. Mestan, Molecular interactions between the highly selective pan-bcr-abl inhibitor, AMN107, and the tyrosine kinase domain of Abl, Blood 106 (11) (2005) 3365.

### Example 6

### Safety in Healthy Volunteers

All vodobatinib doses were generally safe and well tolerated. Eleven subjects (58%) reported 28 TEAEs. Four subjects reported seven treatment-related TEAEs. TEAEs unlikely related to study treatment were reported in 21% and 37% of subjects respectively. The TEAEs were mild (58% of subjects) to moderate (21% of subjects) in intensity and none were more intense. There were no SAEs. No fatal or unknown outcomes were reported. Only one subject reported one TEAE that had not resolved by the end of the study. Overall, no clinically relevant or significant findings were observed related to clinical laboratory results, vital signs, ECGs, or physical examinations. The most common TEAEs (>10%) were post lumbar puncture syndrome (42.1%), headache and nausea (15.8%), abdominal distention (10.5%) and back pain (10.5%).

### Pharmacokinetics in Healthy Volunteers and PD Patients (CSF and Plasma)

A summary of vodobatinib plasma PK parameters in healthy volunteers (as determined in example 2) and PD patients (as determined in example 3) by dose cohort is presented in Table 10. Demographics were similar across cohorts.

TABLE 10
Plasma PK of Vodobatinib in Healthy Subjects and PD Patients
following Once-Daily Oral Dosing with Vodobatinib Capsules

| | | | Cₘₐₓ (ng/mL) | | | | [symbol] | | | AUC₀₋₂₄ (ng*h/mL) | | | |
|---|---|---|---|---|---|---|---|---|---|---|---|---|---|
| | | | Healthy Subjects | | | | | | | | | | |
| Dose (mg) | | N | Day 1 | | Day 7 | | Day 1 | | Day 7 | Day 1 | | Day 7 | |
| 48 | mg | 6 | 1378 | (29%) | 1395 | (46%) | 2.8 | (1.5-4.0) | 4.0 (1.5-12.0) | 8871 | (21%) | 12854 | (70%) |
| 192 | mg | 6 | 3775 | (45%) | 5377 | (45%) | 3.5 | (1.6-4.0) | 4.0 (4.0-4.1) | 34474 | (26%) | 44131 | (58%) |
| 384 | mg | 6 | 6632 | (65%) | 7850 | (65%) | 4.0 | (1.5-6.0) | 3.5 (3.5-4.0) | 47606 | (72%) | 54879 | (74%) |

PD patients

| | | | Day 1 | | Day 14 | | Day 1 | | Day 14 | Day 1 | | Day 14 | |
|---|---|---|---|---|---|---|---|---|---|---|---|---|---|
| 6 | mg | 6 | 164 | (69%) | 182 | (94%) | 2.0 | (1.0-24.0) | 2.0 (2.0-4.0) | 794 | (49%) | 1250 | (56%) |
| 12 | mg | 6 | 255 | (38%) | 301 | (57%) | 2.0 | (1.1-24.0) | 2.0 (6.6-2.1) | 2044 | (51%) | 3250 | (77%) |
| 18 | mg | 5^{a} | 410 | (69%) | 309 | (26%) | 2.0 | (1.0-24.0) | 3.0 (0.0-4.0) | 2730 | (32%) | 3371 | (49%) |
| 24 | mg | 9^{b} | 385 | (73%) | 715 | (37%) | 4.0 | (2.0-24.0) | 2.0 (0.5-4 0) | 2689 | (48%) | 5724 | (40%) |
| 48 | mg | 9 | 964 | (63%) | 1110 | (57%) | 4.0 | (1.0-8.0) | 2.0 (1.8-8.0) | 5308 | (43%) | 7912 | (47%) |
| 96 | mg | 10 | 1359 | (53%) | 2803 | (64%) | 4.0 | (2.0-24.0 | 2.0 (0.0-4.0) | 10176 | (29%) | 17129 | (57%) |
| 192 | mg | 6^{c} | 1526 | (54%) | 3234 | (80%) | 2.0 | (1.3-13.1) | 1.0 (0.0-4.0) | 18629 | (43%) | 33788 | (62%) |
| 384 | mg | 6^{c} | 3023 | (58%) | 4512 | (36%) | 4.1 | (1.3-8.1) | 1.0 (0.0-8.0) | 29874 | (55%) | 43521 | (41%) |

^{a}N = 4 on D 14;
^{b}N = 8 on D 14;
^{c}N = 5 on D 14;
Values are presented as Mean (CV%), except Tmax which is presented as median (range). [symbol]
indicates data missing or illegible when filed

After the initial dose on Day 1, plasma vodobatinib concentrations increased with dose with peak concentrations (Cₘₐₓ) achieved within 2.8-4.0 h. The mean AUC0-24 values increased in approximate proportion to dose between 48 and 192 mg, whereas a less than proportional increase was observed between the 192 and 384 mg cohort. A similar trend in dose proportionality was observed on Day 7 as well. Following once-daily administration for 7 days, the mean accumulation ratios (Day 7/Day 1) of Cₘₐₓ and AUC₀₋₂₄ ranged from 1.1 to 1.6 across the doses. On Day 7, quantifiable CSF concentrations of vodobatinib were observed in all subjects in the three dose cohorts. The mean CSF AUC₀₋₂₄ values increased from 8.9 h*ng/mL (18.3 h*nM) for the 48 mg cohort to 59.2 h*ng/mL (130.4 h*nM) and 54.5 h*ng/mL (120.1 h*nM) for the 192 and 384 mg cohorts, respectively (Table 11A). The lack of dose related increase in CSF levels at 384 mg was consistent with a similar less than proportional increase in vodobatinib plasma exposures seen in the 192 mg and 384 mg cohorts.

Importantly, the mean CSF concentrations of vodobatinib remained well above its human c-Abl IC₅₀ of 0.9 nM for the entire 24-h dosing cycle for individuals on daily doses of either 192 mg or 384 mg vodobatinib.

As shown in Table 11A, both the vodobatinib Cₘₐₓ, CSF and C_{avg}, CSF following daily doses of either 192 mg or 384 mg vodobatinib capsules resulted in concentrations that exceeded the IC50 for human c-Abl.

TABLE 11A
CSF Concentrations of Vodobatinib following Once-Daily Oral
Dosing with Vodobatinib - (Day 7, Healthy Volunteers)

| | Dose | C_{max, CSF} | AUC₀₋₂₄ | C_{avg, CSF} ^{a} | C_{max, CSF}/ | C_{avg, CSF}/ |
|---|---|---|---|---|---|---|
| | (mg) | (ng/ml) | (ng*h/mL) | (ng/mL) | IC₅₀^{b} | IC₅₀^{b} |
| Vodobatinib [symbol] | 48 | 0.8 | 10.0 | 0.42 | 2.0 | 1.0 |
| | | (1.8 nM) | | (0.9 nM) | | |
| Vodobatinib | 192 | 5.2 | 74.5 | 3.3 | 13 | 7.6 |
| | | (11.5 nM) | | (6.8 nM) | | |
| | 384 | 5.5 | 58.7 | 2.4 | 14 | 6.0 |
| | | (12.3 nM) | | (5.3 nM) | | |

^{a}C_{avg, CSF} was estimated as AUC_{(0-24)/24}.
^{b}IC₅₀ value of 0.9 nM for vodobatinib was used. [symbol]
indicates data missing or illegible when filed

Based on the published literature, nilotinib is a less potent c-Abl inhibitor than vodobatinib (Table 9), and as previously described it only achieved steady state CSF levels lower than its c-Abl IC50. As shown in Table 11A, at the 192 and 384 mg vodobatinib doses, the mean vodobatinib Cₘₐₓ, CSF (11-12 nM; 5.2-5.5 ng/mL) was approximately 13-fold higher than its c-Abl IC50 (0.9 nM). In contrast based on PK estimates in the published literature for nilotinib [1,16](summarized in Table 11B), at the 300 mg dose the mean nilotinib Cₘₐₓ, CSF (2.6-4.2 nM) is approximately 4- to 17-fold lower than its c-Abl IC50 of 15-45 nM [22, 23]. At the 192 and 384 mg vodobatinib doses, the mean Vodobatinib C_{avg}, CSF of (5-7 nM) was approximately 6- to 8-fold higher than its c-Abl IC50 (0.9 nM), whereas published C_{avg}, CSF concentrations of nilotinib at 300 mg (0.8 nM) are approximately 20- to 60-fold lower than the c-Abl IC50 of nilotinib.

TABLE 11B
CSF Concentrations of Nilotinib following Once-Daily
Oral Dosing with Nilotinib (3 months, PD Subjects)

| | Dose | C_{max, CSF} | AUC₀₋₂₄ | C_{avg, CSF} ^{a} | C_{max, CSF}/ | C_{avg, CSF}/ |
|---|---|---|---|---|---|---|
| | (mg) | (ng/mL) | (ng*h/mL) | (ng/mL) | IC₅₀ [symbol] | IC₅₀ [symbol] |
| Nilotinib | 150 | 0.6 | NR | NA | 0.03-0.1 | NA |
| H | | (1.5 nM) | | | | |
| | 300 | 1.4 | NR | NA | 0.06-0.18 | NA |
| | | (2.6 nM) | | | | |
| Nilotinib | 150 | 1.0 | 6.157 | 0.26 | 0.04-0.13 | 0.01-0.03 |
| I | | (1.9 nM)^{b} | | (0.5 nM) | | |
| | 300 | 2.2 | 9.56 | 0.40 | 0.09-0.28 | 0.02-0.05 |
| | | (4.1 nM)^{b} | | (0.8 nM) | | |

NA—Not applicable; NR—Not reported.
^{a}C_{avg, CSF} was estimated as AUC_{(0-24)/24}.
^{b}Concentration reported in the literature ¹ as nM (1.85 nM at 150 mg and 4.12 nM at 300 mg) was converted into ng/mL using molecular weight of 529.5 Da.
^{c}IC₅₀ values of 15 nM^{A} to 45 nM^{B} for nilotinib were used. [symbol]
indicates data missing or illegible when filed

A: T. O'Hare, D. K. Walters, E. P. Stoffregen, T. Jia, P. W. Manley, J. Mestan, S. W. Cowan-Jacob, F. Y. Lee, M. C. Heinrich, M. W. N. Deininger, B. J. Druker, In vitro activity of Bcr-Abl inhibitors AMN107 and BMS-354825 against clinically relevant imatinib-resistant Abl kinase domain mutants, Cancer Res. 65 (11) (2005 Jun. 1) 4500-4505.
B: U. Rix, O. Hantschel, G. Durnberger, L. L. Remsing, M. Planyavsky, N. V. Fernbach, I. Kaupe, K. L. Bennett, P. Valent, J. Colinge, T. K\ddot{ } ocher, G. Superti-Furga, Chemical proteomic profiles of the BCR-ABL inhibitors imatinib, nilotinib, and dasatinib reveal novel kinase and nonkinase targets, Blood 110 (12) (2021) 4055-4063.
H: T. Simuni, B. Fiske, K. Merchant, C. S. Coffey, E. Klingner, C. Caspell-Garcia, D. E. Lafontant, H. Matthews, R. K. Wyse, P. Brundin, D. K. Simon, M. Schwarzschild, D. Weiner, J. Adams, C. Venuto, T. M. Dawson, L. Baker, M. Kostrzebski, T. Ward, G. Rafaloff, Parkinson Study Group NILO-PD Investigators and Collaborators, Efficacy of Nilotinib in patients with moderately advanced Parkinson disease: a randomized clinical trial, JAMA Neurol. 78 (2021) 312-320.
I: F. L. Pagan, M. L. Hebron, B. Wilmarth, Y. Torres-Yaghi, A. Lawler, E. E. Mundel, N. Yusuf, N. J. Starr, M. Anjum, J. Arellano, H. H. Howard, W. Shi, S. Mulki, T. Kurd-Misto, S. Matar, X. Liu, J. Ahn, C. Moussa, Nilotinib effects on safety, tolerability, and potential biomarkers in Parkinson disease: a phase 2 randomized clinical trial, JAMA Neurol. 77 (2020) 309-317.

Safety in PD Patients Study NCT02970019 was a multi-center safety and tolerability study of vodobatinib in 60 subjects (see Table 12). Overall, a total of 32 PD subjects administered various daily doses of vodobatinib for 14 days reported 63 TEAEs. The most common TEAEs (reported in more than one subject in the double-blind portion of the study) were abdominal pain (vodobatinib 96, 192, and 384 mg), blood urine present (vodobatinib 24 mg and placebo), rash (vodobatinib 6, 192, and 384 mg), headache (vodobatinib 6 and 384 mg), and tremor (vodobatinib 6 mg and placebo). The most common treatment related TEAEs occurring in more than one subject were rash, abdominal pain and tremor. These were of Grade 1 severity, except for two events abdominal pain of Grade 2 severity in one subject and rash of Grade 2 severity in one subject. There were no drug-related changes in the QTc interval and no clinically significant drug-related changes in any laboratory values.

TABLE 12
Adverse Events in Parkinson's Disease Patients
treated Daily for 14 Days with Vodobatinib.

| Dose Level | |
|---|---|
| (n/N) | Possibly related AEs by subject |
| 6 mg DB (5/8) | Increased appetite, feet & ankle weakness, infraorbital puffiness, |
| | increased tremor and dry eyes |
| | Increased tremor |
| 12 mg DB (3/8) | Light-headedness |
| | Decreased balance & decreased gait |
| 18 mg DB (3/6) | New onset atrial fibrillation and orthostasis (moderate severity) |
| 24 mg DB (1/8) | None |
| 48 mg DB (2/8) | None |
| 24 mg OL (3/3) | Vertigo, headache, nausea, weakness, vomiting (all moderate |
| | severity) |
| | Headache |
| 96 mg DB (2/8) | Abdominal pain and soft stool |
| | Brain fog, worsening of right lower leg cramps |
| 96 mg OL (2/4) | Dry eyes |
| | Worsening of muscle cramps (“probably related”) |
| 192 mg DB (3/8) | Mild rash, epigastric pain |
| | Somnolence, worsening of left upper extremities resting tremor |
| | Erythematous, pruritic rash on torso and head |

DB—double blind cohort.
OL—open label cohort including CSF collection.
n—number of AEs per N.
N—number of subjects in dose group.
Each bullet represents one subject. Assignment of relatedness as per investigator.

### Pharmacokinetics in PD Patients (CSF and Plasma)

In PD patients as in healthy volunteers, the mean Cₘₐₓ and AUC₀₋₂₄ of vodobatinib in plasma increased with dose from 6 mg to 384 mg (Table 10). The median Tₘₐₓ values ranged from one to 4 h. The mean CSF levels collected at 2-3 h post dose on Day 14 were 0.66 ng/mL (1.5 nM), 0.67 ng/mL (1.5 nM) and 1.1 ng/mL (2.4 nM) at the 24 mg, 48 mg and 96 mg doses, respectively. The concentrations at 48 mg (same dose as tested in healthy and PD patients) in PD patients are comparable to CSF concentrations obtained in healthy subjects dosed with 48 mg vodobatinib, i.e., 0.5-0.7 ng/mL at 2-3 h. Thus, in PD patients there is a high potential for c-Abl inhibition in the CNS. All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

### Example 7

### Phase 2 Study in Early Parkinson's Disease Patients Evaluating the Safety and Efficacy of Vodobatinib

A phase 2, randomized, double-blind, placebo-controlled study of vodobatinib in approximately 504 subjects with early Parkinson's disease is ongoing. The subjects are being administered a low dose (96 mg capsule or 192 mg powder of vodobatinib) or high dose (192 mg capsule or 384 mg powder of vodobatinib). The subjects are initially administered the capsule and then transitioned to the powder. The increase in dose from capsule to powder results not in doubling, but on average an approximately 1.4-fold increase in exposure to vodobatinib. The objective of the study is to determine if vodobatinib reduces the rate of progression of early-stage Parkinson's disease over 40 weeks, as defined by the sum of the MDS-UPDRS (Movement Disorder Society-Unified Parkinson's Disease Rating Scale) Parts 2 and 3 scores.

The study is performed in subjects (i) aged at least 50 years, (ii) having a body mass index (BMI) greater than 18.5 kg/m² and less than 45 kg/m², (iii) diagnosed with “Clinically Probable PD” according to the MDS clinical diagnostic criteria, with documented diagnosis of PD per treating physician's records within three years of the screening visit, (iv) a disease severity according to modified Hoehn & Yahr stage≤2, and (v) projected to not require to start dopaminergic therapy within 9 months from baseline.

While the invention has been described in connection with specific aspects thereof, it will be understood that invention is capable of further modifications and this application is intended to cover any variations, uses, or adaptations following, in general, the principles and including such departures from the present disclosure that come within known or customary practice within the art to which the invention pertains and can be applied to the essential features hereinbefore set forth, and follows in the scope of the claimed.

1. A method of reducing the rate of progression of early-stage Parkinson's disease in a human subject having a disease severity according to modified Hoehn & Yahr stage≤2 comprising administering to the subject an effective amount of a compound of Formula I: 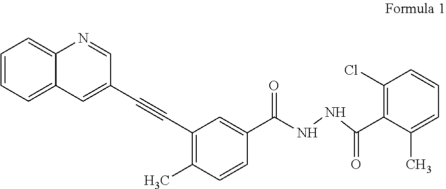 [SMILES: Cc1ccc(C(=O)NNC(=O)c2c(C)cccc2Cl)cc1C#Cc1cnc2ccccc2c1]
or a pharmaceutically acceptable salt thereof.

2-9. (canceled)

10. The method of claim 1, wherein the administration of a compound of Formula I or a pharmaceutically acceptable salt thereof increases the time to significant worsening of the subject on the MDS-UPDRS Parts II and III.

11. (canceled)

12. The method of claim 1, wherein the subject is not concomitantly administering symptomatic medication for Parkinson's disease.

13. The method of claim 1, wherein the subject is administered the compound of Formula I or a pharmaceutically acceptable salt thereof in a fasting state.

14-15. (canceled)

16. The method of claim 1, wherein the subject is aged 50 years or older.

17. (canceled)

18. The method of claim 1, wherein the subject is not concomitantly receiving dopamine replacement medication.

19-20. (canceled)

21. The method of claim 1, wherein the subject is administered about 48 mg to about 480 mg of a compound of Formula I or a pharmaceutically acceptable salt thereof.

22-23. (canceled)

24. The method of claim 1, wherein the subject is administered about 192 mg or about 384 mg of a compound of Formula I or a pharmaceutically acceptable salt thereof.

25. (canceled)

26. The method of claim 1, wherein the subject is not being treated with any dopamine treatment other than monoamine oxidase-B (MAO-B) inhibitors.

27. (canceled)

28. The method of claim 1, wherein the compound of Formula I or a pharmaceutically acceptable salt thereof is administered as one or more solid dosage forms where each solid dosage form comprises (i) polyvinyl caprolactam-polyvinyl acetate-polyethylene glycol graft co-polymer, (ii) colloidal silicon dioxide, (iii) sodium lauryl sulphate, (iv) crospovidone, (v) mannitol, (vi) flavoring agent, and any combination of any of the foregoing.

29. (canceled)

30. The method of claim 1, wherein the subject has been receiving treatment with a monoamine oxidase B (MAOB) inhibitor.

31-33. (canceled)

34. The method of claim 1, wherein the compound of Formula I or a pharmaceutically acceptable salt thereof is administered in an amount sufficient to achieve a mean plasma AUC₀₋₂₄ ranging from about 20,000 ng*h/mL to about 60,000 ng*h/mL.

35. (canceled)

36. The method of claim 1, wherein the compound of Formula I or a pharmaceutically acceptable salt thereof is administered in an amount sufficient to achieve a plasma Cₘₐₓ ranging from about 1800 ng/mL to about 12,000 ng/mL.

37-47. (canceled)

48. A method for the treatment, or delay, inhibition, or suppression of the progression of, Parkinson's disease in a human subject comprising administering to the subject (i) about 192 mg or about 384 mg of a compound of Formula I or a pharmaceutically acceptable salt thereof: 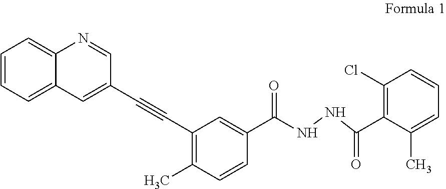 [SMILES: Cc1ccc(C(=O)NNC(=O)c2c(C)cccc2Cl)cc1C#Cc1cnc2ccccc2c1]
in the form of an aqueous suspension or (ii) an amount of a compound of Formula I or a pharmaceutically acceptable salt thereof in one or more solid dosage forms sufficient to achieve the same bioavailability as the aqueous suspension, wherein the a compound of Formula I or a pharmaceutically acceptable salt thereof is administered in an amount sufficient to achieve (i) plasma Cₘₐₓ ranging from about 500 ng/mL to about 15,000 ng/mL, (ii) mean plasma AUC₀₋₂₄ ranging from about 5,000 ng*h/mL to about 75,000 ng*h/mL, or (iii) cerebrospinal fluid Cₘₐₓ ranging from about 0.4 ng/mL to about 7.0 ng/mL, and (iv) mean cerebrospinal fluid C_{avg} from about 0.3 ng/mL to about 4.0 ng/mL.

49-51. (canceled)

52. The method of claim 48, wherein the Parkinson's disease is early-stage Parkinson's disease.

53. The method of claim 1, wherein upon the occurrence of an adverse event, the daily dose of a compound of Formula I or a pharmaceutically acceptable salt thereof is reduced by half.

54-62. (canceled)

63. An oral dosage form for reducing the rate of progression of early-stage Parkinson's disease in a human subject having a disease severity according to modified Hoehn & Yahr stage≤2 wherein the dosage form comprises an effective amount of a compound of Formula I: 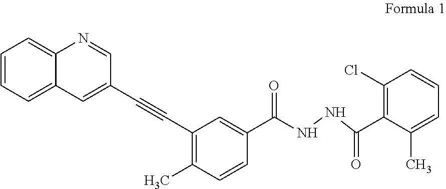 [SMILES: Cc1ccc(C(=O)NNC(=O)c2c(C)cccc2Cl)cc1C#Cc1cnc2ccccc2c1]
or a pharmaceutically acceptable salt thereof.

64-68. (canceled)

69. The oral dosage form as claimed in claim 63, wherein the oral dosage for is selected from a solid oral dosage form or an aqueous suspension.

70. The oral dosage form as claimed in claim 69, wherein the oral dosage form comprises (i) polyvinyl caprolactam-polyvinyl acetate-polyethylene glycol graft co-polymer, (ii) colloidal silicon dioxide, (iii) sodium lauryl sulphate, (iv) crospovidone, (v) mannitol, (vi) flavoring agent, and any combination of any of the foregoing.

71. The oral dosage form as claimed in claim 69, wherein the aqueous suspension is prepared immediately prior to administration by adding a powder comprising a compound of Formula I or a pharmaceutically acceptable salt thereof to water and mixing uniformly with a spoon or other stirrer.
